SEQUENCE LISTING (332 sequences)

SEQ ID NO: 1 - DNA, 31 bp, Artificial Sequence
  Primer
tcgccatagctaagttgtttattgtttccat

SEQ ID NO: 2 - DNA, 30 bp, Artificial Sequence
  Primer
taaggtatgacaccggataaatcatataaa

SEQ ID NO: 3 - DNA, 29 bp, Artificial Sequence
  Primer
taatgggtaaatatcaccctcatggtgac

SEQ ID NO: 4 - DNA, 28 bp, Artificial Sequence
  Primer
tcaccctcatggtgactcatctatttat

SEQ ID NO: 5 - DNA, 23 bp, Artificial Sequence
  Primer
tcttgagccatacgtaccattgc

SEQ ID NO: 6 - DNA, 28 bp, Artificial Sequence
  Primer
tatccattgaaccaaagttaccttggcc

SEQ ID NO: 7 - DNA, 31 bp, Artificial Sequence
  Primer
tagccatacgtaccattgcttcataaataga

SEQ ID NO: 8 - DNA, 2664 bp, Staphylococcus aureus
atggctgaattacctcaatcaagaataaatgaacgaaatattaccagtgaaatgcgtgaatcatttttagattatgcgatgagtgttatcgttgctcgtgcattgccagatgttcgtgacggtttaaaaccagtacatcgtcgtatactatatggattaaatgaacaaggtatgacaccggataaatcatataaaaaatcagcacgtatcgttggtgacgtaatgggtaaatatcaccctcatggtgactcatctatttatgaagcaatggtacgtatggctcaagatttcagttatcgttatccgcttgttgatggccaaggtaactttggttcaatggatggagatggcgcagcagcaatgcgttatactgaagcgcgtatgactaaaatcacacttgaactgttacgtgatattaataaagatacaatagattttatcgataactatgatggtaatgaaagagagccgtcagtcttacctgctcgattccctaacttgttagccaatggtgcatcaggtatcgcggtaggtatggcaacgaatattccaccacataacttaacagaattaatcaatggtgtacttagcttaagtaagaaccctgatatttcaattgctgagttaatggaggatattgaaggtcctgatttccctactgctggacttattttaggtaagagtggtattagacgtgcatatgaaacaggtcgtggttcaattcaaatgcgttctcgtgcagttattgaagaacgtggaggcggacgtcaacgtattgttgtcactgaaattcctttccaagtgaataaggctcgtatgattgaaaaaattgcagaactcgttcgtgacaagaaaattgacggtatcactgatttacgtgatgaaacaagtttacgtactggtgtgcgtgtcgttattgatgtgcgtaaggatgcaaatgctagtgtcattttaaataacttatacaaacaaacgccacttcaaacatcatttggtgtgaatatgattgcacttgtaaatggtagaccgaagcttattaatttaaaagaagcgttggtacattatttagagcatcaaaagacagttgttagaagacgtacgcaatacaacttacgtaaagctaaagatcgtgcccacattttagaaggattacgtatcgcacttgaccatatcgatgaaattatttcaacgattcgtgagtcagatacagataaagttgcaatggaaagcttgcaacaacgcttcaaactttctgaaaaacaagctcaagctattttagacatgcgtttaagacgtctaacaggtttagagagagacaaaattgaagctgaatataatgagttattaaattatattagtgaattagaagcaatcttagctgatgaagaagtgttattacagttagttagagatgaattgactgaaattagagatcgtttcggtgatgatcgtcgtacagaaattcaattaggtggatttgaagacttagaggacgaagacttaattccagaagaacaaatagtaattacactaagccataataactacattaaacgtttgccggtatctacatatcgtgctcaaaaccgtggtggtcgtggtgttcaaggtatgaatacattggaagaagattttgtcagtcaattggtaactttaagtacacatgaccatgtattgttctttactaacaaaggtcgtgtatacaaacttaaaggttatgaagtgcctgagttatcaagacagtctaaaggtattcctgtagtgaatgctattgaacttgaaaatgatgaagtcattagtacaatgattgctgttaaagaccttgaaagtgaagacaacttcttagtgtttgcaactaaacgtggtgtcgttaaacgttcagcattaagtaacttctcaagaataaatagaaatggtaagattgcgatttcgttcagagaagatgatgagttaattgcagttcgcttaacaagtggtcaagaagatatcttgattggtacatcacatgcatcattaattcgattccctgaatcaacattacgtcctttaggccgtacagcaacgggtgtgaaaggtattacacttcgtgaaggtgacgaagttgtagggcttgatgtagctcatgcaaacagtgttgatgaagtattagtagttactgaaaatggttatggtaaacgtacgccagttaatgactatcgtttatcaaatcgtggtggtaaaggtattaaaacagctacgattactgagcgtaatggtaatgttgtatgtatcactacagtaactggtgaagaagatttaatgattgttactaatgcaggtgtcattattcgactagatgttgcagatatttctcaaaatggtcgtgcagcacaaggtgttcgcttaattcgcttaggtgatgatcaatttgtttcaacggttgctaaagtaaaagaagatgcagaagatgaaacgaatgaagatgagcaatctacttcaactgtatctgaagatggtactgaacaacaacgtgaagcggttgtaaatgatgaaacaccaggaaatgcaattcatactgaagtgattgattcagaagaaaatgatgaagatggacgtattgaagtaagacaagatttcatggatcgtgttgaagaagatatacaacaatcatcagatgaagaataa

SEQ ID NO: 9 - DNA, 2664 bp, Staphylococcus aureus
atggctgaattacctcaatcaagaataaatgaacgaaatattaccagtgaaatgcgtgaatcatttttagattatgcgatgagtgttatcgttgctcgtgcattgccagatgttcgtgacggtttaaaaccagtacatcgtcgtatactatatggattaaatgaacaaggtatgacaccggataaatcatataaaaaatcagcacgtatcgttggtgacgtaatgggtaaatatcaccctcatggtgactcatctatttatgaagcaatggtacgtatggctcaagatttcagttatcgttatccgcttgttgatggccaaggtaactttggttcaatggatggagatggcgcagcagcaatgcgttatactgaagcgcgtatgactaaaatcacacttgaactgttacgtgatattaataaagatacaatagattttatcgataactatgatggtaatgaaagagagccgtcagtcttacctgctcgattccctaacttattagccaatggtgcatcaggtatcgcggtaggtatggcaacgaatattccaccacataacttaacagaattaatcaatggtgtacttagcttaagtaagaaccctgatatttcaattgctgagttaatggaggatattgaaggtcctgatttcccaactgctggacttattttaggtaagagtggtattagacgtgcatatgaaacaggtcgtggttcaattcaaatgcgttctcgtgcagttattgaagaacgtggaggcggacgtcaacgtattgttgtcactgaaattcctttccaagtgaataaggctcgtatgattgaaaaaattgcagagctcgttcgtgacaagaaaattgacggtatcactgatttacgtgatgaaacaagtttacgtactggtgtgcgtgtcgttattgatgtgcgtaaggatgcaaatgctagtgtcattttaaataacttatacaaacaaacacctcttcaaacatcatttggtgtgaatatgattgcacttgtaaatggtagaccgaagcttattaatttaaaagaagcgttggtacattatttagagcatcaaaagacagttgttagaagacgtacgcaatacaacttacgtaaagctaaagatcgtgcccacattttagaaggattacgtatcgcacttgaccatatcgatgaaattatttcaacgattcgtgagtcagatacagataaagttgcaatggaaagcttgcaacaacgcttcaaactttctgaaaaacaagctcaagctattttagacatgcgtttaagacgtctaacaggtttagagagagacaaaattgaagctgaatataatgagttattaaattatattagtgaattagaagcaatcttagctgatgaagaagtgttattacagttagttagagatgaattgactgaaattagagatcgtttcggtgatgatcgtcgtacagaaattcaattaggtggatttgaaaacttagaggacgaagacttaattccagaagaacaaatagtaattacactaagccataataactacattaaacgtttgccggtatctacatatcgtgctcaaaaccgtggtggtcgtggtgttcaaggtatgaatacattggaagaagattttgtcagtcaattggtaactttaagtacacatgaccatgtattgttctttactaacaaaggtcgtgtatacaaacttaaaggttacgaagtgcctgagttatcaagacagtctaaaggtattcctgtagtgaatgctattgaacttgaaaatgatgaagtcattagtacaatgattgctgttaaagaccttgaaagtgaagacaacttcttagtgtttgcaactaaacgtggtgtcgttaaacgttcagcattaagtaacttctcaagaataaatagaaatggtaagattgcgatttcgttcagagaagatgatgagttaattgcagttcgcttaacaagtggtcaagaagatatcttgattggtacatcacatgcatcattaattcgattccctgaatcaacattacgtcctttaggccgtacagcaacgggtgtgaaaggtattacacttcgtgaaggtgacgaagttgtagggcttgatgtagctcatgcaaacagtgttgatgaagtattagtagttactgaaaatggttatggtaaacgtacgccagttaatgactatcgcttatcaaatcgtggtggtaaaggtattaaaacagctacgattactgagcgtaatggtaatgttgtatgtatcactacagtaactggtgaagaagatttaatgattgttactaatgcaggtgtcattattcgactagatgttgcagatatttctcaaaatggtcgtgcagcacaaggtgttcgcttaattcgcttaggtgatgatcaatttgtttcaacggttgctaaagtaaaagaagatgcagaagatgaaacgaatgaagatgagcaatctacttcaactgtatctgaagatggtactgaacaacaacgtgaagcggttgtaaatgatgaaacaccaggaaatgcaattcatactgaagtgattgattcagaagaaaatgatgaagatggacgtattgaagtaagacaagatttcatggatcgtgttgaagaagatatacaacaatcatcagatgaagaataa

SEQ ID NO: 10 - DNA, 178 bp, Staphylococcus aureus
caaggtatgacaccggataaatcatataaaaaatcagcacgtatcgttggtgacgtaatgggtaaatatcaccctcatggtgactcatctatttatgaagcaatggtacgtatggctcaagatttcagttatcgttatccgcttgttgatggccaaggtaactttggttcaatggatg

SEQ ID NO: 11 - DNA, 96 bp, Staphylococcus aureus
caaggtatgacaccggataaatcatataaaaaatcagcacgtatcgttggtgacgtaatgggtaaatatcaccctcatggtgactcatctatttat

SEQ ID NO: 12 - DNA, 91 bp, Staphylococcus aureus
tctatttatgaagcaatggtacgtatggctcaagatttcagttatcgttatccgcttgttgatggccaaggtaactttggttcaatggatg

SEQ ID NO: 13 - DNA, 27 bp, Artificial Sequence
  Primer
tttccgatgcaacgtaatgagatttca

SEQ ID NO: 14 - DNA, 26 bp, Artificial Sequence
  Primer
tcgtatgaccagcttcggtactacta

SEQ ID NO: 15 - DNA, 27 bp, Artificial Sequence
  Primer
tttatgaccagcttcggtactactaaa

SEQ ID NO: 16 - DNA, 25 bp, Artificial Sequence
  Primer
tttcctccttttgaaagcgacggtt

SEQ ID NO: 17 - DNA, 24 bp, Artificial Sequence
  Primer
ttcctccttttgaaagcgacggtt

SEQ ID NO: 18 - DNA, 32 bp, Artificial Sequence
  Primer
tcctttgatatattatgcgatggaaggttggt

SEQ ID NO: 19 - DNA, 24 bp, Artificial Sequence
  Primer
tgggctctttctcgcttaaacacc

SEQ ID NO: 20 - DNA, 25 bp, Artificial Sequence
  Primer
tgggctctttctcgcttaaacacct

SEQ ID NO: 21 - DNA, 31 bp, Artificial Sequence
  Primer
taattgggctctttctcgcttaaacacctta

SEQ ID NO: 22 - DNA, 29 bp, Artificial Sequence
  Primer
tagataattgggctctttctcgcttaaac

SEQ ID NO: 23 - DNA, 26 bp, Artificial Sequence
  Primer
tacgatttcacttccgcagccagatt

SEQ ID NO: 24 - DNA, 31 bp, Artificial Sequence
  Primer
tgcgtacaatacgctttatgaaattttaaca

SEQ ID NO: 25 - DNA, 31 bp, Artificial Sequence
  Primer
taatcaagcattggaagatgaaatgcatacc

SEQ ID NO: 26 - DNA, 31 bp, Artificial Sequence
  Primer
tgacatggactccccctatataactcttgag

SEQ ID NO: 27 - DNA, 29 bp, Artificial Sequence
  Primer
tgatcgttgagaagggatttgcgaaaaga

SEQ ID NO: 28 - DNA, 27 bp, Artificial Sequence
  Primer
tcgctatcttatcgttgagaagggatt

SEQ ID NO: 29 - DNA, 24 bp, Artificial Sequence
  Primer
tgcaaaatctgcaacgagctttgg

SEQ ID NO: 30 - DNA, 28 bp, Artificial Sequence
  Primer
tcatcctaagccaagtgtagactctgta

SEQ ID NO: 31 - DNA, 29 bp, Artificial Sequence
  Primer
tataagtgggtaaaccgtgaatatcgtgt

SEQ ID NO: 32 - DNA, 25 bp, Artificial Sequence
  Primer
tctttgaaatcggctcaggaaaagg

SEQ ID NO: 33 - DNA, 31 bp, Artificial Sequence
  Primer
tcatgataatatctttgaaatcggctcagga

SEQ ID NO: 34 - DNA, 25 bp, Artificial Sequence
  Primer
tcaggaaaagggcattttacccttg

SEQ ID NO: 35 - DNA, 32 bp, Artificial Sequence
  Primer
tctgaacatgataatatctttgaaatcggctc

SEQ ID NO: 36 - DNA, 24 bp, Artificial Sequence
  Primer
taatcgtggaatacgggtttgcta

SEQ ID NO: 37 - DNA, 30 bp, Artificial Sequence
  Primer
tggaacaaaatagtctctcggattttgact

SEQ ID NO: 38 - DNA, 24 bp, Artificial Sequence
  Primer
tcggaatctgatgttgcagttgtt

SEQ ID NO: 39 - DNA, 26 bp, Artificial Sequence
  Primer
tgagctgcatcaactgtattggatag

SEQ ID NO: 40 - DNA, 29 bp, Artificial Sequence
  Primer
tgagtaacatccatatttctgccatacgt

SEQ ID NO: 41 - DNA, 24 bp, Artificial Sequence
  Primer
tccaatgccacaaactcgtgaaca

SEQ ID NO: 42 - DNA, 26 bp, Artificial Sequence
  Primer
tcccaggtgacgatgtacctgtaatc

SEQ ID NO: 43 - DNA, 21 bp, Artificial Sequence
  Primer
tacaggccgtgttgaacgtgg

SEQ ID NO: 44 - DNA, 23 bp, Artificial Sequence
  Primer
tgccgtgttgaacgtggtcaaat

SEQ ID NO: 45 - DNA, 27 bp, Artificial Sequence
  Primer
tcgtgttgaacgtggtcaaatcaaagt

SEQ ID NO: 46 - DNA, 29 bp, Artificial Sequence
  Primer
tgttgaacgtggtcaaatcaaagttggtg

SEQ ID NO: 47 - DNA, 30 bp, Artificial Sequence
  Primer
tgaacgtggtcaaatcaaagttggtgaaga

SEQ ID NO: 48 - DNA, 31 bp, Artificial Sequence
  Primer
tgaacgtggtcaaatcaaagttggtgaagaa

SEQ ID NO: 49 - DNA, 27 bp, Artificial Sequence
  Primer
tgtggtcaaatcaaagttggtgaagaa

SEQ ID NO: 50 - DNA, 25 bp, Artificial Sequence
  Primer
tggtcaaatcaaagttggtgaagaa

SEQ ID NO: 51 - DNA, 28 bp, Artificial Sequence
  Primer
tgaaggtggacgtcacactccattcttc

SEQ ID NO: 52 - DNA, 23 bp, Artificial Sequence
  Primer
tcctgaagcaagtgcatttacga

SEQ ID NO: 53 - DNA, 29 bp, Artificial Sequence
  Primer
tccttatagggatggctatcagtaatgtt

SEQ ID NO: 54 - DNA, 26 bp, Artificial Sequence
  Primer
tcagcaaatgcatcacaaacagataa

SEQ ID NO: 55 - DNA, 30 bp, Artificial Sequence
  Primer
tacaaaggtcaaccaatgacattcagacta

SEQ ID NO: 56 - DNA, 26 bp, Artificial Sequence
  Primer
tgaagtagaaatgactgaacgtccga

SEQ ID NO: 57 - DNA, 29 bp, Artificial Sequence
  Primer
taaaacaaactacggtaacattgatcgca

SEQ ID NO: 58 - DNA, 24 bp, Artificial Sequence
  Primer
tcaggtactgctatccaccctcaa

SEQ ID NO: 59 - DNA, 21 bp, Artificial Sequence
  Primer
tgtactgctatccaccctcaa

SEQ ID NO: 60 - DNA, 11 bp, Artificial Sequence
  Primer
tccaccctcaa

SEQ ID NO: 61 - DNA, 30 bp, Artificial Sequence
  Primer
tcaccaggttcaactcaaaaaatattaaca

SEQ ID NO: 62 - DNA, 28 bp, Artificial Sequence
  Primer
tttacacatatcgtgagcaatgaactga

SEQ ID NO: 63 - DNA, 24 bp, Artificial Sequence
  Primer
tcacatatcgtgagcaatgaactg

SEQ ID NO: 64 - DNA, 27 bp, Artificial Sequence
  Primer
tgggcgtgagcaatgaactgattatac

SEQ ID NO: 65 - DNA, 28 bp, Artificial Sequence
  Primer
tggacacatatcgtgagcaatgaactga

SEQ ID NO: 66 - DNA, 31 bp, Artificial Sequence
  Primer
tggtttagataattccttaggatctatgcgt

SEQ ID NO: 67 - DNA, 27 bp, Artificial Sequence
  Primer
tgcgtataaaaaacacagatggcagca

SEQ ID NO: 68 - DNA, 29 bp, Artificial Sequence
  Primer
tccaaataagtggcgttacaaatactgaa

SEQ ID NO: 69 - DNA, 29 bp, Artificial Sequence
  Primer
tcttttacaaaaggggaaaaagttgactt

SEQ ID NO: 70 - DNA, 28 bp, Artificial Sequence
  Primer
tcgtcatcagctaactcaaatacatgga

SEQ ID NO: 71 - DNA, 32 bp, Artificial Sequence
  Primer
tctgaaatgaatagtgatagaactgtaggcac

SEQ ID NO: 72 - DNA, 31 bp, Artificial Sequence
  Primer
tgaatagtgatagaactgtaggcacaatcgt

SEQ ID NO: 73 - DNA, 27 bp, Artificial Sequence
  Primer
tagtgatagaactgtaggcacaatcgt

SEQ ID NO: 74 - DNA, 32 bp, Artificial Sequence
  Primer
ttggtcctttttatacgaaagaagaagttgaa

SEQ ID NO: 75 - DNA, 22 bp, Artificial Sequence
  Primer
tcgccggcaatgccattggata

SEQ ID NO: 76 - DNA, 29 bp, Artificial Sequence
  Primer
tgatggcaagtggatagggtataatacag

SEQ ID NO: 77 - DNA, 26 bp, Artificial Sequence
  Primer
tggcaagtggatagggtataatacag

SEQ ID NO: 78 - DNA, 26 bp, Artificial Sequence
  Primer
tggcgagtggatagggtataatacag

SEQ ID NO: 79 - DNA, 33 bp, Artificial Sequence
  Primer
tggggctttaaatattccaattgaagattttca

SEQ ID NO: 80 - DNA, 23 bp, Artificial Sequence
  Primer
ttgcgaatagaacgatggctcgt

SEQ ID NO: 81 - DNA, 32 bp, Artificial Sequence
  Primer
tacttttttaaaactagggatgcgtttgaagc

SEQ ID NO: 82 - DNA, 28 bp, Artificial Sequence
  Primer
tgaagtagaaggtgcaaagcaagttaga

SEQ ID NO: 83 - DNA, 29 bp, Artificial Sequence
  Primer
tcacctccaagtttagatcacttgagaga

SEQ ID NO: 84 - DNA, 31 bp, Artificial Sequence
  Primer
tgaattagttcaatcatttgttgaacgacgt

SEQ ID NO: 85 - DNA, 29 bp, Artificial Sequence
  Primer
tacaatgcttgtttatgctggtaaagcag

SEQ ID NO: 86 - DNA, 27 bp, Artificial Sequence
  Primer
tcttgtttatgctggtaaagcagatgg

SEQ ID NO: 87 - DNA, 27 bp, Artificial Sequence
  Primer
tcttgtttatgctggtaaagcagatgg

SEQ ID NO: 88 - DNA, 29 bp, Artificial Sequence
  Primer
tccaaaccaggtgtatcaagaacatcagg

SEQ ID NO: 89 - DNA, 30 bp, Artificial Sequence
  Primer
tgcaagttaagaaagctgttgcaggtttat

SEQ ID NO: 90 - DNA, 34 bp, Artificial Sequence
  Primer
tcccacgaaacagatgaagaaattaacaaaaaag

SEQ ID NO: 91 - DNA, 29 bp, Artificial Sequence
  Primer
tcaaactgggcaatcggaactggtaaatc

SEQ ID NO: 92 - DNA, 26 bp, Artificial Sequence
  Primer
tccagcacgaattgctgctatgaaag

SEQ ID NO: 93 - DNA, 26 bp, Artificial Sequence
  Primer
tgaattgctgctatgaaaggtggctt

SEQ ID NO: 94 - DNA, 26 bp, Artificial Sequence
  Primer
tagctggcggtatggagaatatgtct

SEQ ID NO: 95 - DNA, 34 bp, Artificial Sequence
  Primer
tacaacatattattaaagagacgggtttgaatcc

SEQ ID NO: 96 - DNA, 27 bp, Artificial Sequence
  Primer
tgataatgaagggaaacctttttcacg

SEQ ID NO: 97 - DNA, 28 bp, Artificial Sequence
  Primer
tcgatcgtgactctctttattttcagtt

SEQ ID NO: 98 - DNA, 31 bp, Artificial Sequence
  Primer
tgtaattaaccgaaggttctgtagaagtatg

SEQ ID NO: 99 - DNA, 29 bp, Artificial Sequence
  Primer
tatatgaacaataccagttccttctgagt

SEQ ID NO: 100 - DNA, 29 bp, Artificial Sequence
  Primer
tgagctggtgctatatgaacaataccagt

SEQ ID NO: 101 - DNA, 30 bp, Artificial Sequence
  Primer
tcccttccttaatatgagaaggaaaccact

SEQ ID NO: 102 - DNA, 22 bp, Artificial Sequence
  Primer
tggctgcggaagtgaaatcgta

SEQ ID NO: 103 - DNA, 23 bp, Artificial Sequence
  Primer
tctggctgcggaagtgaaatcgt

SEQ ID NO: 104 - DNA, 27 bp, Artificial Sequence
  Primer
ttaatctggctgcggaagtgaaatcgt

SEQ ID NO: 105 - DNA, 25 bp, Artificial Sequence
  Primer
taatctggctgcggaagtgaaatcg

SEQ ID NO: 106 - DNA, 23 bp, Artificial Sequence
  Primer
taatctggctgcggaagtgaaat

SEQ ID NO: 107 - DNA, 28 bp, Artificial Sequence
  Primer
tcgttaattaatctggctgcggaagtga

SEQ ID NO: 108 - DNA, 31 bp, Artificial Sequence
  Primer
tggtatattcgttaattaatctggctgcgga

SEQ ID NO: 109 - DNA, 26 bp, Artificial Sequence
  Primer
tcgtcctctcgaatctccgatatacc

SEQ ID NO: 110 - DNA, 30 bp, Artificial Sequence
  Primer
tcagatataaatggaacaaatggagccact

SEQ ID NO: 111 - DNA, 24 bp, Artificial Sequence
  Primer
tctgcatttttgcgagcctgtcta

SEQ ID NO: 112 - DNA, 31 bp, Artificial Sequence
  Primer
tgtacaataaggagtcaccttatgtccctta

SEQ ID NO: 113 - DNA, 32 bp, Artificial Sequence
  Primer
tcttggcttaggatgaaaatatagtggtggta

SEQ ID NO: 114 - DNA, 27 bp, Artificial Sequence
  Primer
tgagtctacacttggcttaggatgaaa

SEQ ID NO: 115 - DNA, 30 bp, Artificial Sequence
  Primer
tcaatacagagtctacacttggcttaggat

SEQ ID NO: 116 - DNA, 30 bp, Artificial Sequence
  Primer
tggacgatattcacggtttacccacttata

SEQ ID NO: 117 - DNA, 26 bp, Artificial Sequence
  Primer
ttgacatttgcatgcttcaaagcctg

SEQ ID NO: 118 - DNA, 30 bp, Artificial Sequence
  Primer
tgatggtctatttcaatggcagttacgaaa

SEQ ID NO: 119 - DNA, 30 bp, Artificial Sequence
  Primer
tttatggtctatttcaatggcagttacgaa

SEQ ID NO: 120 - DNA, 27 bp, Artificial Sequence
  Primer
tatggtctatttcaatggcagttacga

SEQ ID NO: 121 - DNA, 34 bp, Artificial Sequence
  Primer
tccgtagttttgcataatttatggtctatttcaa

SEQ ID NO: 122 - DNA, 28 bp, Artificial Sequence
  Primer
tcaacttctgccattaaaagtaatgcca

SEQ ID NO: 123 - DNA, 31 bp, Artificial Sequence
  Primer
tcattaggtaaaatgtctggacatgatccaa

SEQ ID NO: 124 - DNA, 29 bp, Artificial Sequence
  Primer
tcacacctgtaagtgagaaaaaggttgat

SEQ ID NO: 125 - DNA, 30 bp, Artificial Sequence
  Primer
tggaaaactcatgaaattaaagtgaaagga

SEQ ID NO: 126 - DNA, 29 bp, Artificial Sequence
  Primer
tctcatgaaaaaggctcaggagatacaag

SEQ ID NO: 127 - DNA, 36 bp, Artificial Sequence
  Primer
ttccatttcaactaattctaataattcttcatcgtc

SEQ ID NO: 128 - DNA, 30 bp, Artificial Sequence
  Primer
tggtttgtcagaatcacgttctggagttgg

SEQ ID NO: 129 - DNA, 32 bp, Artificial Sequence
  Primer
tcagcgtagtctaataatttacggaacatttc

SEQ ID NO: 130 - DNA, 31 bp, Artificial Sequence
  Primer
tgcttcagcgtagtctaataatttacggaac

SEQ ID NO: 131 - DNA, 35 bp, Artificial Sequence
  Primer
tcttcagcgtagtctaataatttacggaacatttc

SEQ ID NO: 132 - DNA, 28 bp, Artificial Sequence
  Primer
tgtcaccagcttcagcgtagtctaataa

SEQ ID NO: 133 - DNA, 34 bp, Artificial Sequence
  Primer
tcaccagcttcagcgtagtctaataatttacgga

SEQ ID NO: 134 - DNA, 30 bp, Artificial Sequence
  Primer
tgcgtagtctaataatttacggaacatttc

SEQ ID NO: 135 - DNA, 31 bp, Artificial Sequence
  Primer
taggcataaccatttcagtaccttctggtaa

SEQ ID NO: 136 - DNA, 27 bp, Artificial Sequence
  Primer
tacgctaagccacgtccatatttatca

SEQ ID NO: 137 - DNA, 25 bp, Artificial Sequence
  Primer
tgtttgtgatgcatttgctgagcta

SEQ ID NO: 138 - DNA, 29 bp, Artificial Sequence
  Primer
tagttgaagttgcactatatactgttgga

SEQ ID NO: 139 - DNA, 26 bp, Artificial Sequence
  Primer
taaatgcacttgcttcagggccatat

SEQ ID NO: 140 - DNA, 30 bp, Artificial Sequence
  Primer
tgatcctgaatgtttatatctttaacgcct

SEQ ID NO: 141 - DNA, 27 bp, Artificial Sequence
  Primer
tcccaatctaacttccacataccatct

SEQ ID NO: 142 - DNA, 27 bp, Artificial Sequence
  Primer
tggatagacgtcatatgaaggtgtgct

SEQ ID NO: 143 - DNA, 25 bp, Artificial Sequence
  Primer
tattcttcgttactcatgccataca

SEQ ID NO: 144 - DNA, 11 bp, Artificial Sequence
  Primer
tactcatgcca

SEQ ID NO: 145 - DNA, 11 bp, Artificial Sequence
  Primer
tattcttcgtt

SEQ ID NO: 146 - DNA, 29 bp, Artificial Sequence
  Primer
taaccaccccaagatttatctttttgcca

SEQ ID NO: 147 - DNA, 28 bp, Artificial Sequence
  Primer
ttgtgatatggaggtgtagaaggtgtta

SEQ ID NO: 148 - DNA, 24 bp, Artificial Sequence
  Primer
tgtgatatggaggtgtagaaggtg

SEQ ID NO: 149 - DNA, 30 bp, Artificial Sequence
  Primer
tgggatggaggtgtagaaggtgttatcatc

SEQ ID NO: 150 - DNA, 33 bp, Artificial Sequence
  Primer
tggggatatggaggtgtagaaggtgttatcatc

SEQ ID NO: 151 - DNA, 28 bp, Artificial Sequence
  Primer
tgtaaaagcagggctataataaggactc

SEQ ID NO: 152 - DNA, 25 bp, Artificial Sequence
  Primer
tgcccttttgtaaaagcagggctat

SEQ ID NO: 153 - DNA, 31 bp, Artificial Sequence
  Primer
tactttaaggggctatctttaccatgaacct

SEQ ID NO: 154 - DNA, 27 bp, Artificial Sequence
  Primer
taagttccttcgctagtatgttggctt

SEQ ID NO: 155 - DNA, 28 bp, Artificial Sequence
  Primer
tcactttgatatgtggatccgtcattca

SEQ ID NO: 156 - DNA, 32 bp, Artificial Sequence
  Primer
tcttcttctttcgtataaaaaggaccaattgg

SEQ ID NO: 157 - DNA, 31 bp, Artificial Sequence
  Primer
tcttctttcgtataaaaaggaccaattggtt

SEQ ID NO: 158 - DNA, 29 bp, Artificial Sequence
  Primer
tgcgctaattcttcaacttcttctttcgt

SEQ ID NO: 159 - DNA, 26 bp, Artificial Sequence
  Primer
tctttcgtataaaaaggaccaattgg

SEQ ID NO: 160 - DNA, 32 bp, Artificial Sequence
  Primer
tggtgttctagtatagattgaggtagtggtga

SEQ ID NO: 161 - DNA, 32 bp, Artificial Sequence
  Primer
tgagttaaaatgcgattgatttcagtttccaa

SEQ ID NO: 162 - DNA, 28 bp, Artificial Sequence
  Primer
taagcaatacctttacttgcaccacctg

SEQ ID NO: 163 - DNA, 27 bp, Artificial Sequence
  Primer
taagcaatacctttacttgcaccacct

SEQ ID NO: 164 - DNA, 25 bp, Artificial Sequence
  Primer
taagcaatacctttacttgcaccac

SEQ ID NO: 165 - DNA, 29 bp, Artificial Sequence
  Primer
ttcataagcaatacctttacttgcaccac

SEQ ID NO: 166 - DNA, 27 bp, Artificial Sequence
  Primer
tacctgcattaatcgcttgttcatcaa

SEQ ID NO: 167 - DNA, 30 bp, Artificial Sequence
  Primer
tcgaattcagctaaatacttttcagcatct

SEQ ID NO: 168 - DNA, 32 bp, Artificial Sequence
  Primer
tgatattgaactggtgtaccataatagttgcc

SEQ ID NO: 169 - DNA, 29 bp, Artificial Sequence
  Primer
tcgctctctcaagtgatctaaacttggag

SEQ ID NO: 170 - DNA, 30 bp, Artificial Sequence
  Primer
tgggacgtaatcgtataaattcatcatttc

SEQ ID NO: 171 - DNA, 29 bp, Artificial Sequence
  Primer
tgcattgtaccgaagtagttcacattgtt

SEQ ID NO: 172 - DNA, 32 bp, Artificial Sequence
  Primer
tggtacacctggtttcgttttgatgatttgta

SEQ ID NO: 173 - DNA, 30 bp, Artificial Sequence
  Primer
tgttcttgatacacctggtttcgttttgat

SEQ ID NO: 174 - DNA, 29 bp, Artificial Sequence
  Primer
tacacctggtttcgttttgatgatttgta

SEQ ID NO: 175 - DNA, 30 bp, Artificial Sequence
  Primer
tgttctggattgattgcacaatcaccaaag

SEQ ID NO: 176 - DNA, 31 bp, Artificial Sequence
  Primer
tgagatgttgatgatttaccagttccgattg

SEQ ID NO: 177 - DNA, 33 bp, Artificial Sequence
  Primer
tggtacaacatcgttagctttaccactttcacg

SEQ ID NO: 178 - DNA, 34 bp, Artificial Sequence
  Primer
tggcagcaatagtttgacgtacaaatgcacacat

SEQ ID NO: 179 - DNA, 29 bp, Artificial Sequence
  Primer
tccaacccagaaccacatactttattcac

SEQ ID NO: 180 - DNA, 26 bp, Artificial Sequence
  Primer
tcgccagctagcacgatgtcattttc

SEQ ID NO: 181 - DNA, 33 bp, Artificial Sequence
  Primer
tccatctgttaaaccatcatataccatgctatc

SEQ ID NO: 182 - DNA, 26 bp, Artificial Sequence
  Primer
ttcgtgctggattttgtccttgtcct

SEQ ID NO: 183 - DNA, 26 bp, Artificial Sequence
  Primer
tggtggtgaaatagataggactgctt

SEQ ID NO: 184 - DNA, 22 bp, Artificial Sequence
  Primer
tggaggtgtcactccacacgaa

SEQ ID NO: 185 - DNA, 22 bp, Artificial Sequence
  Primer
ttgcacaagcaaggcgctattt

SEQ ID NO: 186 - DNA, 26 bp, Artificial Sequence
  Primer
tggatgttaagggtgattttcccgaa

SEQ ID NO: 187 - DNA, 26 bp, Artificial Sequence
  Primer
tgtggagtaacactgcatgaaaacaa

SEQ ID NO: 188 - DNA, 26 bp, Artificial Sequence
  Primer
tagcatcagaactgttgttccgctag

SEQ ID NO: 189 - DNA, 29 bp, Artificial Sequence
  Primer
taaccattcaagaactagatcttcaggca

SEQ ID NO: 190 - DNA, 28 bp, Artificial Sequence
  Primer
tcattcaagaactagatcttcaggcaag

SEQ ID NO: 191 - DNA, 25 bp, Artificial Sequence
  Primer
tcaccagtttgccacgtatcttcaa

SEQ ID NO: 192 - DNA, 28 bp, Artificial Sequence
  Primer
tgagcttttagttgactttttcaacagc

SEQ ID NO: 193 - DNA, 29 bp, Artificial Sequence
  Primer
tttcacacagcgtgtttatagttctacca

SEQ ID NO: 194 - DNA, 27 bp, Artificial Sequence
  Primer
tagcgaatgtggctttacttcacaatt

SEQ ID NO: 195 - DNA, 25 bp, Artificial Sequence
  Primer
atcaatttggtggccaagaacctgg

SEQ ID NO: 196 - DNA, 22 bp, Artificial Sequence
  Primer
ttgactgcggcacaacacggat

SEQ ID NO: 197 - DNA, 26 bp, Artificial Sequence
  Primer
tgctatggtgttaccttccctatgca

SEQ ID NO: 198 - DNA, 30 bp, Artificial Sequence
  Primer
tagcaacaaatatatctgaagcagcgtact

SEQ ID NO: 199 - DNA, 32 bp, Artificial Sequence
  Primer
tgaaaagtatggatttgaacaactcgtgaata

SEQ ID NO: 200 - DNA, 27 bp, Artificial Sequence
  Primer
tcattatcatgcgccaatgagtgcaga

SEQ ID NO: 201 - DNA, 28 bp, Artificial Sequence
  Primer
tttcatcttatcgaggacccgaaatcga

SEQ ID NO: 202 - DNA, 32 bp, Artificial Sequence
  Primer
tgcaccggctattaagaattactttgccaact

SEQ ID NO: 203 - DNA, 25 bp, Artificial Sequence
  Primer
tggatggggattagcggttacaatg

SEQ ID NO: 204 - DNA, 22 bp, Artificial Sequence
  Primer
tagctggcgcgaaattaggtgt

SEQ ID NO: 205 - DNA, 25 bp, Artificial Sequence
  Primer
tgtcggtacacgatattcttcacga

SEQ ID NO: 206 - DNA, 27 bp, Artificial Sequence
  Primer
tgaaatctcattacgttgcatcggaaa

SEQ ID NO: 207 - DNA, 24 bp, Artificial Sequence
  Primer
tctcattacgttgcatcggaaaca

SEQ ID NO: 208 - DNA, 23 bp, Artificial Sequence
  Primer
tagtaccgaagctggtcatacga

SEQ ID NO: 209 - DNA, 21 bp, Artificial Sequence
  Primer
tgcagggaacagctttaggca

SEQ ID NO: 210 - DNA, 27 bp, Artificial Sequence
  Primer
taactctgatgtttttgatgggaaggt

SEQ ID NO: 211 - DNA, 33 bp, Artificial Sequence
  Primer
tgtatggtggtgtaacgttacatgataataatc

SEQ ID NO: 212 - DNA, 30 bp, Artificial Sequence
  Primer
ttgtatgtatggtggtgtaacgttacatga

SEQ ID NO: 213 - DNA, 30 bp, Artificial Sequence
  Primer
ttaacatgaaggaaaccactttgataatgg

SEQ ID NO: 214 - DNA, 30 bp, Artificial Sequence
  Primer
tggaataacaaaacatgaaggaaaccactt

SEQ ID NO: 215 - DNA, 30 bp, Artificial Sequence
  Primer
tgagtttaacagttcaccatatgaaacagg

SEQ ID NO: 216 - DNA, 24 bp, Artificial Sequence
  Primer
tggtatgatatgatgcctgcacca

SEQ ID NO: 217 - DNA, 30 bp, Artificial Sequence
  Primer
tggtgacttcataatggatgaagttgaagt

SEQ ID NO: 218 - DNA, 32 bp, Artificial Sequence
  Primer
tgggattttaaaaaacattggtaacatcgcag

SEQ ID NO: 219 - DNA, 32 bp, Artificial Sequence
  Primer
tcttgcagcagtttatttgatgaacctaaagt

SEQ ID NO: 220 - DNA, 31 bp, Artificial Sequence
  Primer
tgtacccgctgaattaacgaatttatacgac

SEQ ID NO: 221 - DNA, 31 bp, Artificial Sequence
  Primer
tggtattctattttgctgataatgacctcgc

SEQ ID NO: 222 - DNA, 34 bp, Artificial Sequence
  Primer
tggcactcttgcctttaatattagtaaactatca

SEQ ID NO: 223 - DNA, 30 bp, Artificial Sequence
  Primer
tccacttatcgcaaatggaaaattaagcaa

SEQ ID NO: 224 - DNA, 30 bp, Artificial Sequence
  Primer
tgcacttatcgcaaatggaaaattaagcaa

SEQ ID NO: 225 - DNA, 25 bp, Artificial Sequence
  Primer
tgatacttcaacgcctgctgctttc

SEQ ID NO: 226 - DNA, 24 bp, Artificial Sequence
  Primer
tatacttcaacgcctgctgctttc

SEQ ID NO: 227 - DNA, 33 bp, Artificial Sequence
  Primer
tgcaattgctttagttttaagtgcatgtaattc

SEQ ID NO: 228 - DNA, 33 bp, Artificial Sequence
  Primer
tccttgctttagttttaagtgcatgtaattcaa

SEQ ID NO: 229 - DNA, 31 bp, Artificial Sequence
  Primer
tgttgggagtattccttaccatttaagcaca

SEQ ID NO: 230 - DNA, 24 bp, Artificial Sequence
  Primer
tggaaagccatgcgtctgacatct

SEQ ID NO: 231 - DNA, 26 bp, Artificial Sequence
  Primer
tggatattcaccgaacactagggttg

SEQ ID NO: 232 - DNA, 23 bp, Artificial Sequence
  Primer
taagctgccagcggaatgctttc

SEQ ID NO: 233 - DNA, 30 bp, Artificial Sequence
  Primer
tttacactacttttattcattgccctaacg

SEQ ID NO: 234 - DNA, 30 bp, Artificial Sequence
  Primer
tgatcatccgtggtataacgatttattagt

SEQ ID NO: 235 - DNA, 29 bp, Artificial Sequence
  Primer
tgacatgataataaccgattgaccgaaga

SEQ ID NO: 236 - DNA, 26 bp, Artificial Sequence
  Primer
tgttcaagagctagatcttcaggcaa

SEQ ID NO: 237 - DNA, 25 bp, Artificial Sequence
  Primer
tgttcaagagctagatcttcaggca

SEQ ID NO: 238 - DNA, 24 bp, Artificial Sequence
  Primer
tctggaggcacaccaaataaaaca

SEQ ID NO: 239 - DNA, 24 bp, Artificial Sequence
  Primer
ttgcaactgctgatttagctcaga

SEQ ID NO: 240 - DNA, 27 bp, Artificial Sequence
  Primer
tagaaatcaaggtgatagtggcaatga

SEQ ID NO: 241 - DNA, 30 bp, Artificial Sequence
  Primer
tctgaatgtctatatggaggtacaacacta

SEQ ID NO: 242 - DNA, 30 bp, Artificial Sequence
  Primer
ttctgaatgtctatatggaggtacaacact

SEQ ID NO: 243 - DNA, 26 bp, Artificial Sequence
  Primer
tcaactcgaattttcaacaggtacca

SEQ ID NO: 244 - DNA, 28 bp, Artificial Sequence
  Primer
ttcaacaggtaccaatgatttgatctca

SEQ ID NO: 245 - DNA, 29 bp, Artificial Sequence
  Primer
tgatctcagaatctaataattgggacgaa

SEQ ID NO: 246 - DNA, 31 bp, Artificial Sequence
  Primer
tctcaaggtgatattggtgtaggtaacttaa

SEQ ID NO: 247 - DNA, 30 bp, Artificial Sequence
  Primer
tttcacatgtaattttgatattcgcactga

SEQ ID NO: 248 - DNA, 30 bp, Artificial Sequence
  Primer
tatttcacatgtaattttgatattcgcact

SEQ ID NO: 249 - DNA, 29 bp, Artificial Sequence
  Primer
taacaactcgccttatgaaacgggatata

SEQ ID NO: 250 - DNA, 27 bp, Artificial Sequence
  Primer
ttgtatgtatggtggtgtaactgagca

SEQ ID NO: 251 - DNA, 27 bp, Artificial Sequence
  Primer
tgctcaacccgatcctaaattagacga

SEQ ID NO: 252 - DNA, 29 bp, Artificial Sequence
  Primer
tggacaatagacaatcacttggatttaca

SEQ ID NO: 253 - DNA, 25 bp, Artificial Sequence
  Primer
tggaggttgttgtatgtatggtggt

SEQ ID NO: 254 - DNA, 25 bp, Artificial Sequence
  Primer
tacaaagcaagacactggctcacta

SEQ ID NO: 255 - DNA, 19 bp, Artificial Sequence
  Primer
gaggaaagtccatgctcac

SEQ ID NO: 256 - DNA, 19 bp, Artificial Sequence
  Primer
gaggaaagtccatgctcgc

SEQ ID NO: 257 - DNA, 17 bp, Artificial Sequence
  Primer
gaggaaagtccgggctc

SEQ ID NO: 258 - DNA, 30 bp, Artificial Sequence
  Primer
tgtacaccatttatccacaaattgattggt

SEQ ID NO: 259 - DNA, 32 bp, Artificial Sequence
  Primer
tgggcaccatttatccacaaattgattggtat

SEQ ID NO: 260 - DNA, 24 bp, Artificial Sequence
  Primer
tcgcgctgtatttttcctccgaga

SEQ ID NO: 261 - DNA, 27 bp, Artificial Sequence
  Primer
tgtcaatatgaaggtgctctgtggata

SEQ ID NO: 262 - DNA, 24 bp, Artificial Sequence
  Primer
tctagcggaacaacagttctgatg

SEQ ID NO: 263 - DNA, 30 bp, Artificial Sequence
  Primer
tcctgaagatctagttcttgaatggttact

SEQ ID NO: 264 - DNA, 32 bp, Artificial Sequence
  Primer
tagtcctttctgaattttaccatcaaaggtac

SEQ ID NO: 265 - DNA, 29 bp, Artificial Sequence
  Primer
tcaggtatgaaacacgattagtcctttct

SEQ ID NO: 266 - DNA, 24 bp, Artificial Sequence
  Primer
acctgcatccctaaacgtacttgc

SEQ ID NO: 267 - DNA, 32 bp, Artificial Sequence
  Primer
tacttcagcttcgtccaataaaaaatcacaat

SEQ ID NO: 268 - DNA, 29 bp, Artificial Sequence
  Primer
tgtaggcaagtgcataagaaattgataca

SEQ ID NO: 269 - DNA, 29 bp, Artificial Sequence
  Primer
tgcaagggaaacctagaattacaaaccct

SEQ ID NO: 270 - DNA, 26 bp, Artificial Sequence
  Primer
tgcatagggaaggtaacaccatagtt

SEQ ID NO: 271 - DNA, 28 bp, Artificial Sequence
  Primer
taacaacgttaccttcgcgatccactaa

SEQ ID NO: 272 - DNA, 28 bp, Artificial Sequence
  Primer
tgttgtgccgcagtcaaatatctaaata

SEQ ID NO: 273 - DNA, 29 bp, Artificial Sequence
  Primer
tgtgaagaactttcaaatctgtgaatcca

SEQ ID NO: 274 - DNA, 28 bp, Artificial Sequence
  Primer
tcttcttgaaaaattgttgtcccgaaac

SEQ ID NO: 275 - DNA, 31 bp, Artificial Sequence
  Primer
tggactaataacaatgagctcattgtactga

SEQ ID NO: 276 - DNA, 31 bp, Artificial Sequence
  Primer
tgaatatgtaatgcaaaccagtctttgtcat

SEQ ID NO: 277 - DNA, 33 bp, Artificial Sequence
  Primer
tgcaacaattaatgctccgacaattaaaggatt

SEQ ID NO: 278 - DNA, 28 bp, Artificial Sequence
  Primer
taaagacaccgctgggtttaaatgtgca

SEQ ID NO: 279 - DNA, 37 bp, Artificial Sequence
  Primer
tcaccgataaataaaatacctaaagttaatgccattg

SEQ ID NO: 280 - DNA, 29 bp, Artificial Sequence
  Primer
tggccacttttatcagcaaccttacagtc

SEQ ID NO: 281 - DNA, 32 bp, Artificial Sequence
  Primer
tagtcttttggaacaccgtctttaattaaagt

SEQ ID NO: 282 - DNA, 30 bp, Artificial Sequence
  Primer
tggaacaccgtctttaattaaagtatctcc

SEQ ID NO: 283 - DNA, 31 bp, Artificial Sequence
  Primer
tcttttctttgcttaattttccatttgcgat

SEQ ID NO: 284 - DNA, 34 bp, Artificial Sequence
  Primer
ttacttccttaccacttttagtatctaaagcata

SEQ ID NO: 285 - DNA, 31 bp, Artificial Sequence
  Primer
tggggacttccttaccacttttagtatctaa

SEQ ID NO: 286 - DNA, 29 bp, Artificial Sequence
  Primer
tcaacaatcagatagatgtcagacgcatg

SEQ ID NO: 287 - DNA, 23 bp, Artificial Sequence
  Primer
tgcaagagcaaccctagtgttcg

SEQ ID NO: 288 - DNA, 23 bp, Artificial Sequence
  Primer
taggatgaaagcattccgctggc

SEQ ID NO: 289 - DNA, 25 bp, Artificial Sequence
  Primer
tcatctgtggtatggcgggtaagtt

SEQ ID NO: 290 - DNA, 28 bp, Artificial Sequence
  Primer
tcatttatttcttcgcttttctcgctac

SEQ ID NO: 291 - DNA, 31 bp, Artificial Sequence
  Primer
taagcaccatataagtctacttttttccctt

SEQ ID NO: 292 - DNA, 33 bp, Artificial Sequence
  Primer
tccccatttaataattccacctactatcacact

SEQ ID NO: 293 - DNA, 30 bp, Artificial Sequence
  Primer
tggtacttcaacttcatccattatgaagtc

SEQ ID NO: 294 - DNA, 30 bp, Artificial Sequence
  Primer
tagctatcttatcgttgagaagggatttgc

SEQ ID NO: 295 - DNA, 28 bp, Artificial Sequence
  Primer
tgagcatttttatatccatctccaccat

SEQ ID NO: 296 - DNA, 32 bp, Artificial Sequence
  Primer
tgcgctatcaacgattttgacaatatatgtga

SEQ ID NO: 297 - DNA, 27 bp, Artificial Sequence
  Primer
tcccatacctatggcgataactgtcat

SEQ ID NO: 298 - DNA, 27 bp, Artificial Sequence
  Primer
taccatctacccaaacattagcaccaa

SEQ ID NO: 299 - DNA, 23 bp, Artificial Sequence
  Primer
tagcaccaatcaccctttcctgt

SEQ ID NO: 300 - DNA, 28 bp, Artificial Sequence
  Primer
tcacaaggaccattataatcaatgccaa

SEQ ID NO: 301 - DNA, 28 bp, Artificial Sequence
  Primer
tgtacaaggaccattataatcaatgcca

SEQ ID NO: 302 - DNA, 26 bp, Artificial Sequence
  Primer
tctggcccctccatacatgtatttag

SEQ ID NO: 303 - DNA, 27 bp, Artificial Sequence
  Primer
tgggtaggtttttatctgtgacgcctt

SEQ ID NO: 304 - DNA, 26 bp, Artificial Sequence
  Primer
tcatctggtttaggatctggttgact

SEQ ID NO: 305 - DNA, 24 bp, Artificial Sequence
  Primer
tgcaactcatctggtttaggatct

SEQ ID NO: 306 - DNA, 24 bp, Artificial Sequence
  Primer
tgtgcaggcatcatgtcataccaa

SEQ ID NO: 307 - DNA, 28 bp, Artificial Sequence
  Primer
ttaccatcttcaaatacccgaacagtaa

SEQ ID NO: 308 - DNA, 26 bp, Artificial Sequence
  Primer
taactcctcttccttcaacaggtgga

SEQ ID NO: 309 - DNA, 32 bp, Artificial Sequence
  Primer
tgctttgtaatctagttcctgaatagtaacca

SEQ ID NO: 310 - DNA, 29 bp, Artificial Sequence
  Primer
tgtctattgtcgattgttacctgtacagt

SEQ ID NO: 311 - DNA, 28 bp, Artificial Sequence
  Primer
tgattcaaatgcagaaccatcaaactcg

SEQ ID NO: 312 - DNA, 22 bp, Artificial Sequence
  Primer
ataagccatgttctgttccatc

SEQ ID NO: 313 - DNA, 18 bp, Artificial Sequence
  Primer
ataagccgggttctgtcg

SEQ ID NO: 314 - DNA, 22 bp, Artificial Sequence
  Primer
gtaagccatgttttgttccatc

SEQ ID NO: 315 - DNA, 2670 bp, Staphylococcus aureus
atggctgaattacctcaatcaagaataaatgaacgaaatattaccagtgaaatgcgtgaatcatttttagattatgcgatgagtgttatcgttgctcgtgcattgccagatgttcgtgacggtttaaaaccagtacatcgtcgtatactatatggattaaatgaacaaggtatgacaccggataaatcatataaaaaatcagcacgtatcgttggtgacgtaatgggtaaatatcaccctcatggtgactcatctatttatgaagcaatggtacgtatggctcaagatttcagttatcgttatccgcttgttgatggccaaggtaactttggttcaatggatggagatggcgcagcagcaatgcgttatactgaagcgcgtatgactaaaatcacacttgaactgttacgtgatattaataaagatacaatagattttatcgataactatgatggtaatgaaagagagccgtcagtcttacctgctcgattccctaacttgttagccaatggagcatcaggtatagcggtaggtatggcaacgaatattccaccacataacttaacagaattaatcaatggtgtacttagcttaagtaagaaccctgatatttcaattgctgagttaatggaggatattgaaggtcctgatttcccaactgctggacttattttaggtaagagtggtattagacgtgcatatgaaacaggtcgtggttcaattcaaatgcgttctcgtgcagttattgaagaacgtggaggcggacgtcaacgtattgttgtcactgaaattcctttccaagtgaataaggctcgtatgattgaaaaaattgcagagctcgttcgtgacaagaaaattgacggtatcactgatttacgtgatgaaacaagtttacgtactggtgtgcgtgtcgttattgatgtgcgtaaggatgcaaatgctagtgtcattttaaataacttatacaaacaaacacctcttcaaacatcatttggtgtgaatatgattgcacttgtaaatggtagaccgaagcttattaatttaaaagaagcgttggtacattatttagagcatcaaaagacagttgttagaagacgtacgcaatacaacttacgtaaagctaaagatcgtgcccacattttagaaggattacgtatcgcacttgaccatatcgatgaaattatttcaacgattcgtgagtcagatacagataaagttgcaatggaaagcttgcaacaacgcttcaaactttctgaaaaacaagctcaagctattttagacatgcgtttaagacgtctaacaggtttagagagagacaaaattgaagctgaatataatgagttattaaattatattagtgaattagaaacaatcttagctgatgaagaagtattactacaattagttagagatgaattaacagaaattcgagatcgtttcggtgatgatcgtcgtactgaaatccaattaggtggatttgaagatttagaagatgaagatctcattccagaagaacaaattgtaattacactaagccataataactacattaaacgtttgccggtatctacatatcgtgctcaaaaccgtggtggtcgtggtgttcaaggtatgaatacattggaagaagattttgtcagtcaattggtaactttaagtacacatgaccatgtattgttctttactaacaaaggtcgtgtatacaaacttaaaggttatgaagtgcctgagttatcaagacagtctaaaggtattcctgtagtgaatgctattgaacttgaaaatgatgaagtcattagtacaatgattgctgttaaagaccttgaaagtgaagacaacttcttagtgtttgcaactaaacgtggtgtcgttaaacgttcagcattaagtaacttctcaagaataaatagaaatggtaagattgcgatttcgttcagagaagatgatgagttaattgcagttcgcttaacaagtggtcaagaagatatcttgattggtacatcacatgcatcattaattcgattccctgaatcaacattacgtcctttaggccgtacagcaacgggtgtgaaaggtattacacttcgtgaaggtgacgaagttgtagggcttgatgtagctcatgcaaacagtgttgatgaagtattagtagttactgaaaatggttatggtaaacgtacgccagttaatgactatcgtttatcaaatcgtggtggtaaaggtattaaaacagctacgattactgagcgtaatggtaatgttgtatgtatcactacagtaactggtgaagaagatttaatgattgttactaatgcaggtgtcattattcgactagatgttgcagatatttctcaaaatggtcgtgcagcacaaggtgttcgcttaattcgcttaggtgatgatcaatttgtttcaacggttgctaaagtaaaagaagatgcagaagatgaaacgaatgaagatgagcaatctacttcaactgtatctgaagatggtactgaacaacaacgtgaagcggttgtaaatgatgaaacaccaggaaatgcaattcatactgaagtgattgattcagaagaaaatgatgaagatggacgtattgaagtaagacaagatttcatggatcgtgttgaagaagatatacaacaatcatcagatgaagatgaagaataa

SEQ ID NO: 316 - DNA, 2682 bp, Staphylococcus aureus
atggctgaattacctcaatcaagaattaatgaacgaaatataaccagtgaaatgcgtgaatcattcttagactatgctatgagtgttatcgtttctcgtgcattacctgatgttagagacggattaaagccagtacatcgtcgtattctttatggtttaaatgaacaaggtatgacgcccgataaaccttataagaaatctgcacgtatagtcggggatgtcatgggtaaatatcaccctcatggtgattcttcaatttatgaagcaatggtaagaatggcccaagactttagttatcgttatccacttgtagatggtcaaggtaactttggctctatggatggtgacggtgcagccgcaatgcgttataccgaagcacgtatgactaaaataacattagaacttttacgtgatatcaacaaagacacaattgattttattgacaactatgatggtaatgaaagagagccgtcagtcttacctgcacgtttccctaacttactagtaaatggtgcggcaggaattgccgtaggtatggctacaaatattcctccccacaatttaactgaagttattgatggtgtgctcagtttaagtaagaatccagacatcacaattaatgagctgatggaagacatccaaggtcctgattttcctacagccggtttagtactaggaaaaagtggtattcgtcgagcttatgaaacaggtcgtgggtcaattcaaatgcgttctcgtgctgaaatagaagaacgtggtggtggccgtcaacgtattgtcgtaacggaaatacctttccaagtcaataaagcgcgtatgattgaaaaaatcgcagagttagttagagataagaaaatcgacggtattacagatttacgtgatgaaacaagtttgcgtacaggtgtaagagtagttattgatgtacgtaaagatgcaaatgcgagtgttattttaaataatttatataaacaaacgccattacaaacatcatttggtgtaaatatgattgctttagtgaatggtagacctaaactaatcaatttaaaagaagcacttattcattacttagaacaccaaaaaacagtggttagacgacgtactgaatataatcttaaaaaagcaagagaccgtgcacatattctagaaggtttacgaatagcactagatcatattgatgaaattatcacaacaattcgtgaatcggacactgataaaattgcgatggcaagtttacaagagcgttttaaacttactgaacgtcaagctcaagcaattttagatatgcgtttaagacgtttaactggattagaaagagataaaatagaatctgagtataatgaacttctagaatatattaaagagttagaagagattttagctgatgaagaagtactattacaattagttcgtgatgaattgactgaaattaaagaacgtttcggcgatgaacgtcgcactgaaattcaattaggtggtctagaagatcttgaagatgaagacttaatccctgaagaacaaattgttattacattaagtcataataactatattaaacgtttaccagtatctacatatcgttctcaaaatcgtggtggtcgtggcatacaaggtatgaacacgttggatgaggacttcgttagtcaattggtaacaatgagtacacatgatcatgttctgttctttacgaataaaggtcgtgtatataaactcaaaggttatgaagttcctgagttgtcacgtcaatccaaaggcatacctattattaatgcgattgaactcgaaaatgacgaaacaataagtacgatgattgcagttaaagaccttgaaagtgaagaagattatctcgtatttgcgacaaaacaaggtatcgttaaacgttcatcattaagtaacttctcccgtattaacaaaaacggtaaaattgcaattaactttaaagaagatgatgaattaattgcagtacgtctaacaacaggtaatgaagatattcttattggaactgcacatgcatcattaattagattctctgaatctacattacgcccattaggccgtacagcagcaggtgtgaaaggtatttctctacgtgaaggggatactgtcgtaggtcttgatgttgcagattcagaaagtgaagatgaagtattagtagttactgaaaatggttacggtaaacgtacacctgttagcgaatatcgtttatcaaatcgtggtggtaaaggaatcaaaactgcgacaattaccgagcgtaatggtaacatcgtttgtatcacaactgtaaccggtgaagaggatttaatggttgtaactaacgctggtgttattattcgtcttgacgttcatgatatttctcaaaatggacgtgcagcacaaggtgtacgccttatgaaactcggagatggtcaatttgtttctactgttgctaaagtaaacgaagaagacgataatgaggaaaatgcagatgaagcgcaacaatctactactactgaaacagcagatgtagaagaggtagtcgatgatcagacaccaggcaatgcgattcatacagaaggtgatgcagaaatggaatctgtagagtctcctgaaaatgatgatcgtattgatattagacaagattttatggatagagtgaatgaagatatcgagagtgcttcagataatgaagaagatagtgatgaataa

SEQ ID NO: 317 - DNA, 29 bp, Artificial Sequence
  Primer
taaccgtttccaaaggtactgtattttgt

SEQ ID NO: 318 - DNA, 34 bp, Artificial Sequence
  Primer
taaccgtttccaaaggtactgtattttgtttacc

SEQ ID NO: 319 - DNA, 28 bp, Artificial Sequence
  Primer
tgagtttgcacttcaaaagaaattgtgt

SEQ ID NO: 320 - DNA, 29 bp, Artificial Sequence
  Primer
tcagtttgcacttcaaaagaaattgtgtt

SEQ ID NO: 321 - DNA, 22 bp, Artificial Sequence
  Primer
tcgcctggtgcaggcatcatat

SEQ ID NO: 322 - DNA, 34 bp, Artificial Sequence
  Primer
tcttcacacttttagaatcaaccgttttattgtc

SEQ ID NO: 323 - DNA, 30 bp, Artificial Sequence
  Primer
tctataggtactgtagtttgttttccgtct

SEQ ID NO: 324 - DNA, 22 bp, Artificial Sequence
  Primer
tttgcaccttaccgccaaagct

SEQ ID NO: 325 - DNA, 22 bp, Artificial Sequence
  Primer
taccttaccgccaaagctgtct

SEQ ID NO: 326 - DNA, 28 bp, Artificial Sequence
  Primer
tccgtctatccacaagttaattggtact

SEQ ID NO: 327 - DNA, 30 bp, Artificial Sequence
  Primer
tagtgttgtacctccatatagacattcaga

SEQ ID NO: 328 - DNA, 24 bp, Artificial Sequence
  Primer
ttctgagctaaatcagcagttgca

SEQ ID NO: 329 - DNA, 23 bp, Artificial Sequence
  Primer
taagccctttgttgcttgcgaca

SEQ ID NO: 330 - DNA, 28 bp, Artificial Sequence
  Primer
tcagacccactactataccagtctagca

SEQ ID NO: 331 - DNA, 24 bp, Artificial Sequence
  Primer
tgccaacatactagcgaaggaact

SEQ ID NO: 332 - DNA, 31 bp, Artificial Sequence
  Primer
tcccatgaaccttaacttttaaaggtagttc

(19) United States
(12) Patent Application Publication
(10) Pub. No.: US 2010/0204266 A1
(43) Pub. Date: Aug. 12, 2010

(54) COMPOSITIONS FOR USE IN IDENTIFICATION OF MIXED POPULATIONS OF BIOAGENTS

(75) Inventors: David J. Ecker, Encinitas, CA (US); Rangarajan Sampath, San Diego, CA (US); Christian Massire, Carlsbad, CA (US)

Correspondence Address: Casimir Jones, S.C., 2275 Deming Way, Suite 310, Madison, WI, 53562, US

(73) Assignee: Ibis Biosciences, INC, Carlsbad, CA (US)

(21) Appl. No.: 12/532,803

(22) PCT Filed: Mar. 21, 2008
(86) PCT No.: PCT/US08/57904

§ 371 Date: Mar. 22, 2010

Related U.S. Application Data
(60) Provisional application No. 60/896,801, filed on Mar. 23, 2007.

(51) Int. Cl. A61K 31/4375; C12Q 1/68

(52) U.S. Cl. 514/300; 435/6

(57) ABSTRACT

The present invention provides oligonucleotide primers, compositions, and kits containing the same for rapid identification of bacterial bioagents and populations of bioagents which are members of the Staphylococcus bacterial genus by amplification of a segment of bioagent nucleic acid followed by molecular mass analysis.

### CROSS-REFERENCE TO RELATED APPLICATIONS

This application is a U.S. National Phase application under 35 U.S.C. §371 claiming priority to International Application Number PCT/US2008/057904 filed on Mar. 21, 2008 under the Patent Cooperation Treaty, which claims the benefit of priority to U.S. Provisional Application Ser. No. 60/896,801, filed Mar. 23, 2007, the disclosure of which is incorporated by reference in its entirety for any purpose.

### STATEMENT OF GOVERNMENT SUPPORT

This invention was made with support from NIH/NIAID, contract: 1 UC1-A1067232-01, project: 842. The U.S. government has certain rights in the invention.

### SEQUENCE LISTING

Computer-readable forms of the sequence listing, on CD-ROM, containing the file named DIBIS0093WOSEQ.txt, which is 69,632 bytes (measured in MS-DOS), and were created on Mar. 22, 2007, are herein incorporated by reference.

### FIELD OF THE INVENTION

The present invention relates generally to the field of genetic identification and quantification of bioagents, including mixed populations of bioagents and provides methods, compositions and kits useful for this purpose, as well as others, when combined with molecular mass analysis.

### BACKGROUND OF THE INVENTION

Drug resistance is a growing problem in disease treatment and control. Development of antibiotic resistance by bacteria, especially to broad-range antibiotics, is particularly problematic. Resistance emerges as use and/or misuse of drugs provides a selection advantage to resistant populations of infectious bioagents. Effective surveillance of emerging drug resistance is important for identifying, monitoring and controlling resistant populations and for developing appropriate treatment strategies.

Use of drugs to treat infection with bioagents having a propensity towards resistance can lead to treatment failure and/or development of new drug resistance. Furthermore, the methods available for detection of drug resistance can be prohibitively time consuming and often do not provide sufficient sensitivity or precision to detect low percentages of emerging resistant populations of bioagents. Thus, treatment of patients with certain drugs is often avoided, sometimes resulting in over-use of alternative drugs, and/or development of new drug-resistant strains.

Quinolones, specifically fluoroquinolones, are highly potent broad-spectrum antibiotics that are used to treat several types of bacterial infections. Because of their widespread use, resistance to quinolones has become prevalent among several classes of bacterial bioagents. A SNP (single-nucleotide polymorphism) within the quinolone resistance determining region (QRDR) of the gyrA gene confers quinolone resistance to Staphylococcus aureus bacteria. Ciprofloxacin, levofloxacin, moxifloxacin and gatifloxacin, among the fluoroquinolones used in treating certain types of Staphylococcus aureus infections, are being used less frequently in certain types of infections due to the risk of drug-resistance development. Methicillin-resistant Staphylococcus aureus (MRSA) strains are particularly adept at developing quinolone resistance, and are thus not typically treated with quinolones. However, the number of antibiotics available for treating bacteria that are resistant to both methicillin and quinolones is limited. Development of sensitive, rapid methods that would enable early detection of quinolone resistant bacteria might allow for the use of quinolones before resistance emerges.

Standard methods for determining bacterial drug resistance rely on phenotypic characterization. These methods typically require culturing bacteria from a clinical sample for a period of at least 24-48 hours and subsequent susceptibility testing of the cultured bacteria using assays such as agar/broth dilution and/or disk diffusion, which can require an additional 18-24 hours. These tests are relatively insensitive as they rely on visible phenotypic readouts such as culture growth and can only detect a resistant population if it represents a sufficiently high proportion of total bacteria in the sample. Thus, these standard methods are labor intensive, time-consuming, and insensitive, often resulting in misdiagnosis or delay of diagnosis, and by extension, use of inappropriate drug regimens. Thus, there is a long-felt and unmet need for methods that can rapidly detect emerging populations of bioagents and provide sufficient sensitivity and resolution to identify a bioagent that represents only a small percentage of a sample. Specifically, there is a need for methods that can identify small drug-resistant populations in early stages as they emerges in a mixed-population of bioagents, for example, in a sample from a patient being treated with the drug. Such methods would enable monitoring of emerging drug resistance and subsequent design of specific therapeutic approaches tailored to specific bioagent genotypes, and would also reduce the potential for treatment failure and new drug resistance.

### SUMMARY OF THE INVENTION

Provided herein are, inter alia, pairs of primers and compositions comprising pairs of primers; kits comprising the same; and methods for their use in identification of bioagents, populations of bioagents, population genotypes, and mixed populations of bioagents. The forward and reverse primer members of the pairs of primers are configured to amplify nucleic acids from bioagents, thereby generating amplicons for the nucleic acids. In one aspect, the bioagents are comprised within a population of bioagents. In a preferred embodiment, the primer pairs are configured to amplify one or more nucleic acids from each of the bioagents in the population of bioagents. In one embodiment the primers generate bioagent identifying nucleic acid amplicons. The amplicons are preferably generated from portions of nucleic acid sequences that encode genes essential to antibiotic sensitivity and resistance.

The primer pairs each comprise a forward and a reverse primer member. In one embodiment, the primer pair is configured to generate an amplicon from within a region defined by SEQ ID NO.: 10, a region of GenBank gi number 49484912, the QRDR (quinolone resistance determining region) of the gyrA gene within this GenBank gi number. In one aspect, either or both of the primer pair members comprise 20 to 35 nucleobases in length. In one aspect the forward primer pair member comprises at least 70%, at least 80%, at least 90%, at least 95%, at least 97%, or 100% identity to a first portion of SEQ ID NO.: 10. In another aspect, the reverse primer pair member comprises at least 70%, at least 80%, at least 90%, at least 95%, at least 97%, or 100% reverse complementarity to a second portion of SEQ ID NO.: 10. In another embodiment, the forward primer pair member comprises at least 70%, at least 80%, at least 90%, at least 95%, at least 97%, or 100% identity with a portion of SEQ ID NO.: 11, which is a forward primer hybridization region within SEQ ID NO.: 10. In another embodiment, the reverse primer pair member comprises at least 70%, at least 80%, at least 90%, at least 95%, at least 97%, or 100% reverse complementarity with a portion of SEQ ID NO.: 12, a reverse primer hybridization region within SEQ ID NO.: 10. In another aspect, the primer pair members are configured to hybridize with at least 70%, at least 80%, at least 90%, at least 95%, at least 97%, or 100% complementarity within a sequence region of a biogent nucleic acid sequence. In one aspect the bioagent nucleic acid sequence is GenBank gi number 49484912. In another aspect, the bioagent nucleic acid sequence is GenBank gi number 57650036. In another aspect, the bioagent nucleic acid sequence is GenBank gi number 47118324. In another aspect, the bioagent nucleic acid sequence is GenBank gi number 27314460.

In one embodiment, the forward primer pair member comprises SEQ ID NO.:2 with 0-8 nucleobase deletions, additions and/or substitutions. In another embodiment, the forward primer pair member comprises SEQ ID NO.:3 with 0-8 nucleobase deletions, additions and/or substitutions. In another embodiment, the forward primer pair member comprises SEQ ID NO.:4 with 0-8 nucleobase deletions, additions and/or substitutions. In another embodiment, the reverse primer pair member comprises SEQ ID NO.:5 with 0-6 nucleobase deletions, additions and/or substitutions. In another embodiment, the reverse primer pair member comprises SEQ ID NO.: 6 with 0-8 nucleobase deletions, additions and/or substitutions. In another embodiment, the reverse primer pair member comprises SEQ ID NO.: 7 with 0-9 nucleobase deletions, additions and/or substitutions.

In one embodiment, either or both of the primer pair members comprises at least one modified nucleobase. In one aspect the modified nucleobase is a mass modified nucleobase. In one aspect, the mass modified nucleobase is 5-Iodo-C. In another aspect the modified nucleobase is a universal nucleobase. In one aspect, the universal nucleobase is inosine. In another embodiment, either or both of the primer pair members comprise a non-templated 5′ T-residue.

Compositions comprising one or more of the primer pairs and the kits comprising the same, also provided herein, are configured to provide genotyping information, including identification of population genotypes of samples, populations of bioagents, including mixed populations of bioagents.

Also provided herein are methods of identifying one or more bioagents using the primer pairs and/or kits or compositions comprising the same provided herein.

In one embodiment, the methods are performed for identifying a population genotype for a population of bioagents comprised in the sample. In a preferred embodiment, the population of bioagents is a population of bacterial bioagents. In one embodiment, the population of bioagents comprises two or more bioagents from the same genus, the same species, or even the same strain. In one aspect, the two or more bioagents have the same genotype for one or more locus, gene or nucleotide position. In one embodiment, the population of bioagents is a mixed population of bioagents. In this embodiment, two or more of the bioagents in the population are distinguishable based on one or more characteristics. In one example, the two or more bioagents are distinguishable based on two or more distinct genotypes for a gene, locus, or nucleotide position. In one aspect, the distinct genotype confers resistance to one or more drugs or therapeutic agents. In another aspect, the distinct genotype confers sensitivity to one or more drugs or therapeutic agents. In one embodiment, the mixed population of bioagents comprises a plurality of members of the Staphylococcus genus. In a further embodiment, the population of bioagents comprises a plurality of members of the species Staphylococcus aureus. In one embodiment, the population of bioagents comprises a population of bioagents with two or more distinguishable genotypes for a gene that can confer drug resistance or sensitivity. More preferably, the two or more distinguishable genotypes comprise one genotype that confers resistance to quinolones and another genotype that confers sensitivity to quinolones. In a preferred embodiment, the gene that can confer drug resistance is Gyr A. In a preferred aspect, a distinguishable genotype comprises a C→T transition at nucleotide within the Gyr A gene, thereby conferring a leucine in place of a serine for the encoded gyrase protein. In a preferred embodiment, the C→T transition is at nucleotide 251 of a sequence extraction with coordinates 7005-9668 (SEQ ID NO.: 8) of GenBank gi number: 49484912, which comprises a nucleotide sequence encoding Gyr A. In one aspect, one or more genotypes is an emerging genotype. In one aspect, the genotype confers drug resistance. In a preferred aspect, the genotype confers quinolone resistance. In a preferred aspect, the genotype comprises a genotype of the gyrA gene sequence. In one aspect, the genotype comprises a single nucleotide polymorphism.

In one embodiment, the primer pair is preferably configured to generate an amplicon between about 45 and about 200, more preferably, between about 45 and about 192 linked nucleotides in length within at least a portion of the QRDR region (SEQ ID NO.:10) of the Staphylococcus aureus gyrA gene, which confers quinolone resistance or sensitivity. This region comprises the position of the C→T drug resistance-conferring SNP at within the gyrA gene sequence. The SNP, comprising a change of a single “C” nucleobase to a “T” nucleobase, results in a leucine instead of a serine at amino acid position 84 of the protein. In one aspect, the forward primer is configured to comprise sequence identity within SEQ ID NO.: 11, a region of GenBank gi number 49484912, and the reverse primer is configured to comprise reverse complementarity within SEQ ID NO.: 12, another region of GenBank gi number 49484912. The gyrA primer pairs provided herein, when used in the methods provided herein, can detect a single nucleotide change at this SNP position, and are thus able to determine the drug resistant/sensitive genotype for the gyrA gene for a given Staphylococcus aureus bioagent.

In one embodiment, the method is performed on a sample that comprises or is suspected of comprising a bioagent or a population of bioagents. In this embodiment, the method comprises obtaining a sample and amplifying a nucleic acid from each of two or more bioagents in the sample using a primer pair provided herein, thereby generating amplicons from the nucleic acids and determining a molecular mass for each of the amplicons using a mass spectrometer. In a preferred embodiment, the determining using a mass spectrometer is accomplished by electrospray ionization mass spectrometry (ESI-MS). In one aspect, the ESI-MS is Fourier transform ion cyclotron resonance mass spectrometry (FT-ICR-MS). In another aspect, it is time of flight (TOF) mass spectrometry. In another preferred embodiment, the method further comprises calculating a base composition from each molecular mass measurement. In a preferred embodiment, the method further comprises identifying a population genotype for the population of bioagents by comparing each of the molecular mass measurements and/or each of the base compositions calculated from the molecular mass measurements to a database of base compositions and/or molecular masses indexed to the primer pair used in the method and a known bioagent genotype. The database comprises indexed information comprising the molecular mass and/or base composition data that would be derived from a known bioagent having a certain genotype were an amplicon to be generated using the same primer pairs used to amplify nucleic acids in the sample. A match between the experimentally obtained molecular mass and/or base composition obtained by the methods provided herein, for example, on a sample, and a molecular mass and/or base composition comprised in the database correlates a bioagent in the sample with the known bioagent in the database to which the molecular mass and/or base composition is indexed, thus identifying a genotype of that bioagent in the sample. Thus, a sample comprising a population of bioagents that comprises two or more genotypes for the gene or nucleic acid sequence that the primer pair is configured to amplify will correlate with two or more known bioagents in the database. Identification of one or more genotypes by the methods provided herein identifies a population genotype for a population of bioagents.

In one embodiment, the population of bioagents comprises at least two bacteria. In a preferred embodiment, the population of bioagents comprises at least two bacteria belonging to the Staphylococcus genus. More preferably, the population comprises at least two bacteria belonging to the Staphylococcus aureus species. In one preferred aspect, at least one of the at least two bacteria is resistant to quinolone antimicrobial therapy. In another preferred aspect, at least one of the at least two bacteria is sensitive to quinolone antimicrobial therapy. In another preferred aspect, at least one of the at least two bacteria is resistant to quinolone antimicrobial therapy and at least one of the at least two bacteria is sensitive to quinolone antimicrobial therapy.

In one embodiment, an antibiotic regimen is developed that is tailored to treat the identified population genotype for the population of bioagents. In a preferred aspect, the antibiotic regimen tailored to treat the identified genotypes for the population of bioagents is delivered to the sample source. In a preferred embodiment, the sample source is a human subject from whom the sample was taken.

In one embodiment, the steps of the method are periodically repeated. In one aspect, the tailored antibiotic regimen is delivered continuously during the periodic repeating of the steps. In one aspect, the antibiotic regimen is modified after one or more of the periodic repeats of the steps.

Also provided, in one embodiment, are methods for reducing a population of bacteria in a person needing such a treatment. In this embodiment, the sample is obtained from a person suspected of comprising a population of bioagents. In the identifying step of this embodiment, a population genotype is identified in the person. In one aspect, the population of bioagents in the person comprises a single genotype. In another aspect, it comprises a mixed population of bioagents, comprising at least two distinct genotypes. In this embodiment, the method further comprises administering to the person an antibiotic regimen tailored to treat the identified genotypes for the population of bioagents. In this embodiment, preferably, the population of bioagents comprises a population of bacterial bioagents. In one aspect, the steps of obtaining a sample, amplifying, determining, calculating, and identifying are repeated. In one aspect, the tailored antibiotic regimen is delivered continuously during the periodic repeating of the steps. In one aspect, during one or more of the periodic repeats of the method, an emerging genotype is identified in said sample. In this aspect, preferably, the method further comprises modifying the antibiotic regimen to treat the emerging genotype. In one embodiment, the antibiotic regimen comprises an antibiotic for treating quinolone resistant bacteria. In another embodiment, the antibiotic regimen comprises an antibiotic for treating quinolone sensitive bacteria. In another embodiment, the antibiotic regimen comprises an antibiotic for treating quinolone resistant bacteria and an antibiotic for treating quinolone sensitive bacteria. In one aspect, the antibiotic for treating quinolone sensitive bacteria is a quinolone. In one aspect, it is a fluoroquinolone.

Identification of a mixed population of bioagents allows for proper subsequent steps being performed on the sample. In one embodiment, the mixed population of bioagents comprises at least two populations of bioagents; one population that is sensitive to a first antibiotic and another population that is resistant to said first antibiotic. Subsequent steps with such a population can include treatment with a combination of said first antibiotic to reduce the population of the bioagent sensitive thereto, and treatment with a second antibiotic to reduce the population of bioagent that is resistant to said first antibiotic.

In a further embodiment, comparison of experimental data from the sample with the database identifies only a single genotype for the population of bioagents in the sample. In one aspect of this embodiment, subsequent steps can include treatment of the population with a first antibiotic to which the population of bioagents with the one genotype is sensitive. Periodic processing of the sample is then performed as described above, thereby monitoring for the emergence of a population in the sample with a genotype that confers resistance to the administered first antibiotic. In a preferred embodiment, identification of such an emerging drug resistant bioagent or population of drug resistant bioagents is followed by alteration or modification of the treatment regimen to comprise either a second antibiotic or a combination of the first and the second antibiotics. Rapid identification of a population of bioagents in a sample allows for antibiotic regimens to be closely tailored for treatment of the specific bioagents in said sample. Further, the methods provided herein are able to identify bioagents or populations of bioagents that represent small percentages of the total population of bioagents in a sample. Genotypes in mixed populations can be identified with high sensitivity by PCR-ESI/MS because amplified bioagent nucleic acids having different base compositions appear in different positions in the mass spectrum. The dynamic range for mixed PCR-ESI/MS detections has previously been determined to be approximately 100:1 (Hofstadler, S. A. et al., Inter. J. Mass Spectrom. (2005) 242, 23), which allows for detection of genotype variants with as low as 1% abundance in a mixed population. This ability allows early detection of emerging genotypes and emerging populations, including genotypes that confer drug resistance and drug resistant populations.

In one embodiment, one or more of the bioagents comprised in the population of bioagents represents less than 50% of the population of bioagents. In another embodiment, the one or more of the bioagents comprised in the population of bioagents represents less than 25% of the population of bioagents. In another embodiment, one or more of the bioagents represents less than 10% of the population of bioagents. In another embodiment, one or more of the bioagents represents less than 5% of the population of bioagents. In another embodiment, one or more of the bioagents represents less than 4% of the population of bioagents. In another embodiment, one or more of the bioagents represents less than 3% of the population of bioagents. In another embodiment, one or more of the bioagents represents less than 2% of the population of bioagents. In another embodiment, one or more of the bioagents represents between about 1% and about 2% of the population of bioagents. In another embodiment, one or more of the bioagents represents about 1% of the population of bioagents.

In one embodiment, one or more of the genotypes identified by the method represents less than 50% of the population of bioagents. In another embodiment, one or more of the genotypes identified by the methods represents less than 25% of the population of bioagents. In another embodiment, one or more of the genotypes identified by the methods represents less than 15% of the population of bioagents. In another embodiment, one or more of the genotypes identified by the methods represents less than 10% of the population of bioagents. In another embodiment, one or more of the genotypes identified by the methods represents less than 5% of the population of bioagents. In another embodiment, one or more of the genotypes identified by the methods represents less than 4% of the population of bioagents. In another embodiment, one or more of the genotypes identified by the methods represents less than 3% of the population of bioagents. In another embodiment, one or more of the genotypes identified by the methods represents less than 2% of the population of bioagents. In another embodiment, one or more of the genotypes identified by the methods represents between 1 and 2% of the population of bioagents. In another embodiment, one or more of the genotypes identified by the methods represents about 1% of the population of bioagents.

### BRIEF DESCRIPTION OF THE DRAWINGS

The foregoing summary and detailed description is better understood when read in conjunction with the accompanying drawings which are included by way of example and not by way of limitation.

FIG. 1 is a process diagram illustrating a representative primer selection process.

FIG. 2 is a chart showing distribution of Staphylococcus aureus strain identification for 362 clinical isolates obtained using the genotyping primer pair panel and methods described in Example 9.

FIG. 3 shows three spectra obtained using the gyrA primer pair described in Example 13. The top spectrum was generated from a patient (wound) sample, and the bottom two spectra were generated from two different colonies grown from the patient sample. In all spectra, the left peak (or double peak) represents the forward strand of the amplicon, while the right peak (or double peak) represents the reverse strand. The double peaks in the top spectrum are indicative of two different gyrA genotypes present in the patient sample. Thus, the patient sample comprised a mixed population of bioagents. As indicated by dotted lines, one peak in each of the double-peaks corresponds with the middle spectrum, representing a quinolone resistant genotype (Quinolone resistant colony gyrA mutant Ser84>Leu TCA (S)—>TTA (L)), while the other corresponds with the bottom spectrum, representing a quinolone sensitive genotype (Quinolone sensitive colony gyrA wild-type Ser84 TCA). The identification of both quinolone resistant (middle spectrum) and sensitive (bottom spectrum) genotype colonies grown from the sample is further evidence that the double peaks in the top spectrum represent a mixed population in the patient sample. Base compositions determined in this example for each amplicon are shown above each spectrum.

FIG. 4 is a process diagram illustrating an embodiment of the calibration method.

### DETAILED DESCRIPTION OF EMBODIMENTS

As is used herein, a “bioagent” refers to any microorganism or infectious substance, or any naturally occurring, bioengineered or synthesized component of any such microorganism or infectious substance or any nucleic acid derived from any such microorganism or infectious substance. Those of ordinary skill in the art will understand fully what is meant by the term bioagent given the instant disclosure. Preferably, the bioagent is a bacterial bioagent, a bacterium or a nucleic acid derived therefrom. More preferably, the bioagent is a member of the Staphylococcus genus. More preferably still the bioagent is a strain of Staphylococcus aureus. A “population of bioagents” refers to a plurality of bioagents, or at least two bioagents. In some aspects, the population of bioagents is a “mixed population of bioagents,” which comprises two or more distinguishable genotypes for a particular gene, locus or nucleotide position. In other aspects, each bioagent in the plurality of bioagents comprises a single genotype for the gene, locus, or nucleotide position.

As used herein, “primer pairs,” or “oligonucleotide primer pairs” are synonymous terms referring to pairs of oligonucleotides (herein called “primers” or “oligonucleotide primers”) that are configured to bind to conserved sequence regions of a bioagent nucleic acid (that is conserved among two or more bioagents) and to generate bioagent identifying amplicons. The bound primers flank an intervening variable region of the bioagent between the conserved sequence sequences. Upon amplification, the primer pairs yield amplicons that provide base composition variability between two or more bioagents. The variability of the base compositions allows for the identification of one or more individual bioagents from two or more bioagents based on the base composition distinctions. The primer pairs are also configured to generate amplicons that are amenable to molecular mass analysis. Each primer pair comprises two primer pair members. The primer pair members are a “forward primer” (“forward primer pair member,” or “reverse member”), which comprises at least a percentage of sequence identity with the top strand of the reference sequence used in configuring the primer pair, and a “reverse primer” (“reverse primer pair member” or “reverse member”), which comprises at least a percentage of reverse complementarity with the top strand of the reference sequence used in configuring the primer pair. Primer pair configuration is well-known and is described in detail herein.

Primer pair nomenclature, as used herein, includes the identification of a reference sequence. For example, the forward primer for primer pair number 2740 is named GYRA_NC002953_{—}7005-9668_{—}221-249 F. This forward primer name indicates that the forward primer (“_F”) hybridizes to residues 234-261 (“234_{—}261”) of a reference sequence, which in this case is represented by a sequence extraction of coordinates 7005-9668 (SEQ ID NO.: 8) from GenBank gi number 49484912 (corresponding to the version of genbank number NC_{—}002953, as is indicated by the prefix “GYRA_NC002953” and cross-reference in Table 2). In the case of this primer, the reference sequence is the gene within a Staphylococcus aureus genome encoding for GyrA. Primer pair name codes for the primers provided herein are defined in Table 2, which lists gene abbreviations and GenBank gi numbers that correspond with each primer name code.

Sequences of the primers are also provided. One of skill in the art will understand how to determine exact hybridization coordinates of primers with respect to GenBank sequences, given the information provided herein. The primer pairs are selected and configured; however, to hybridize with two or more bioagents. So, the reference sequence in the primer name is used merely to provide a reference, and not to indicate that the primers are selected and configured to hybridize with and generate a bioagent identifying amplicon only from the reference sequence. Rather, the primers hybridize with and generate amplicons from a number of sequences. Further, the sequences of the primer members of the primer pairs are not necessarily fully complementary to the conserved region of the reference bioagent. Rather, the sequences are configured to be “best fit” amongst a plurality of bioagents at these conserved binding sequences. Therefore, the primer members of the primer pairs have substantial complementarity with the conserved regions of the bioagents, including the reference bioagent.

Methods for PCR primer design are well known. One of skill in the art will understand that primer pairs configured to prime amplification of a double stranded sequence are configured and named using one strand of the double stranded sequence as a reference. The forward primer is the primer of the pair that comprises full or partial sequence identity to the one strand of the sequence being used as a reference. The reverse primer is the primer of the pair that comprises reverse complementarity to the one strand of the sequence being used as a reference.

In one embodiment, the “plus” or “top” strand (the primary sequence as submitted to GenBank) of the nucleic acid to which the primers hybridize is used as a reference when designing primer pairs. In this case, the forward primer will comprise identity and the reverse primer will comprise reverse complementarity, to the sequence listed in GenBank for the reference sequence. The ordinarily skilled artisan will understand how to configure primer pairs based upon this disclosure. In some embodiments, the primer pair is configured using the “minus” or “bottom” strand (reverse complement of the primary sequence as submitted to and listed in GenBank). In this case, the forward primer comprises sequence identity to the minus strand, and thus comprises reverse complementarity to the top strand, the sequence listed in GenBank. Similarly, in this case, the reverse primer comprises reverse complementarity to the minus strang, and thus comprises identity to the top strand.

In a preferred embodiment, the primer pairs are configured to generate an amplicon from “within a region of SEQ ID NO.: 10,” which is a specific region of Genbank gi No.: 49484912, a Staphylococcus aureus nucleic acid sequence. Configuring a primer pair to generate an amplicon from “within a region” of a particular nucleic acid reference sequence means that each primer of the pair hybridizes to a portion of the reference sequence that is within that region. One of ordinary skill in the art understands that shifting the coordinates of this region within which the primers hybridize slightly, in one direction or the other, will often result in an equally effective primer pair. Armed with the instant disclosure, one of skill in the art will be able to configure such primer pairs. Thus, in the above mentioned example, a primer pair that hybridizes to a portion of Genbank gi No.: 49484912 that is within a region slightly shifted with respect to SEQ ID NO.: 10 is encompassed by this description.

As is used herein, the term “substantial complementarity” means that a primer member of a primer pair comprises between about 70%-100%, or between about 80-100%, or between about 90-100%, or between about 95-100% identity, or between about 99-100% sequence identity with the conserved binding sequence of any given bioagent. These ranges of identity are inclusive of all whole or partial numbers embraced within the recited range numbers. For example, and not limitation, 75.667%, 82%, 91.2435% and 97% sequence identity are all numbers that fall within the above recited range of 70% to 100%, therefore forming a part of this description.

As used herein, “broad range survey primers” are intelligent primers configured to identify an unknown bioagent as a member of a particular division (e.g., an order, family, class, Glade, or genus). However, in some cases the broad range survey primers are also able to identify unknown bioagents at the species or sub-species level. As used herein, “division-wide primers” are intelligent primers configured to identify a bioagent at the species level and “drill-down” primers are intelligent primers configured to identify a bioagent at the sub-species level. As used herein, the “sub-species” level of identification includes, but is not limited to, strains, subtypes, variants, and isolates. Drill-down primers are not always required for identification at the sub-species level because broad range survey intelligent primers may, in some cases provide sufficient identification resolution to accomplishing this identification objective.

As used herein, the term “conserved region” refers to the region of the bioagent nucleic acid to which the primer pair members are designed to hybridize. Preferably, the conserved region is conserved among two or more bioagents. By the term “highly conserved,” it is meant that the sequence regions exhibit between about 80-100%, or between about 90-100%, or between about 95-100% identity among all, or at least 70%, at least 80%, at least 90%, at least 95%, or at least 99% of species or strains. As used herein, the term “variable region” is used to describe a region that is between the two conserved sequence regions to which the primers of a primer pair hybridize. In other words, the variable region is a region that is flanked by the bound primers of any one primer pair described herein. The region possesses distinct base compositions among at least two bioagents, such that at least one bioagent can be identified at the family, genus, species or sub-species level using the primer pairs and the methods provided herein. The degree of variability between the at least two bioagents need only be sufficient to allow for identification using mass spectrometry or base composition analysis, as described herein. Such a difference can be as slight as a single nucleotide difference occurring between two bioagents. In a preferred embodiment, the variable region is within a reference sequence that comprises an extraction sequence with coordinates 7005-9668 (SEQ ID NO.: 8) of GenBank gi number: 49484912, which comprises a nucleotide sequence encoding gyrase A (GyrA). In another preferred embodiment, the variable region is within the QRDR segment of a gene encoding gyrase A in Staphlylococcus aureus. In a preferred embodiment, this QRDR segment is SEQ ID NO.: 10. In another embodiment, the variable region is within a reference sequence that comprises an extraction sequence with coordinates 7032-9695 (SEQ ID NO.: 9) of GenBank gi number: 57650036, which comprises a nucleotide sequence encoding gyrase A (GyrA). In another embodiment, the variable region is within a reference sequence that comprises an extraction sequence with coordinates 7005-9674 (SEQ ID NO.: 315) of GenBank gi number: 47118324, which comprises a nucleotide sequence encoding gyrase A (GyrA). In another embodiment, the variable region is within a reference sequence that comprises an extraction sequence with coordinates 6916-9597 (SEQ ID NO.: 316) of GenBank gi number: 27314460, which comprises a nucleotide sequence encoding gyrase A (GyrA). In another preferred embodiment the variable region comprises nucleotide position 251 of a gyrA gene in Staphlylococcus aureus. In one aspect, the variable region comprises nucleotide position 251 of the reference sequence that comprises a sequence extraction with coordinates 7005-9668 (SEQ ID NO.: 8) of GenBank gi number: 49484912, which comprises a nucleotide sequence encoding Staphylococcus aureus GyrA.

As used herein, the terms “amplicon” and “bioagent identifying amplicon” refer to a nucleic acid generated using the primer pairs described herein. The amplicon is preferably double stranded DNA; however, it may be RNA and/or DNA:RNA. The amplicon comprises the sequences of the conserved regions/primer pairs and the intervening variable region. Mass spectrometry analysis of the amplicon determines a molecular mass that can be converted into a base composition, or base composition signature for the amplicon. Since the primer pairs provided herein are configured such that two or more different bioagents, when amplified with a given primer pair, will yield amplicons with unique base composition signatures, the base composition signatures can be used to identify bioagents based on association with amplicons. As discussed herein, primer pairs are configured to generate amplicons from two or more bioagents. As such, the base composition of any given amplicon will include the primer pair, the complement of the primer pair, the conserved regions and the variable region from the bioagent that was amplified to generate the amplicon. One skilled in the art understands that the incorporation of the configured primer pair sequences into any amplicon will replace the native bioagent sequences at the primer binding site, and complement thereof. After amplification of the target region using the primers the resultant amplicons having the primer sequences generate the molecular mass data. Amplicons having any native bioagent sequences at the primer binding sites, or complement thereof, are undetectable because of their low abundance. Such is accounted for when identifying one or more bioagents using any particular primer pair. The amplicon further comprises a length that is compatible with mass spectrometry analysis. In one embodiment, bioagent identifying amplicons generate base composition signatures that are unique to the identity or genotype of a bioagent.

Calculation of base composition from a mass spectrometer generated molecular mass becomes increasingly more complex as the length of the amplicon increases. For amplicons comprising unmodified nucleic acid, the upper length as a practical length limit is about 200 consecutive nucleobases. Incorporating modified nucleotides into the amplicon can allow for an increase in this upper limit. In one embodiment, the amplicons generated using any single primer pair will provide sufficient base composition information to allow for identification of at least one bioagent at the family, genus, species or subspecies level. Alternatively, amplicons greater than 200 nucleobases can be generated and then digested to form two or more fragments that are less than 200 nucleobases. Analysis of one or more of the fragments will provide sufficient base composition information to allow for identification of at least one bioagent.

Preferably, amplicons comprise from about 45 to about 200 consecutive nucleobases (i.e., from about 45 to about 200 linked nucleosides). One of ordinary skill in the art will appreciate that this range expressly embodies compounds of 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 nucleobases in length. One ordinarily skilled in the art will further appreciate that the above range is not an absolute limit to the length of an amplicon, but instead represents a preferred length range. Amplicons lengths falling outside of this range are also included herein so long as the amplicon is amenable to calculation of a base composition signature as herein described.

As is used herein, the term “unknown bioagent” can mean either: (i) a bioagent whose existence is not known (for example, the SARS coronavirus was unknown prior to April 2003), which is also called a “true unknown bioagent,” and/or (ii) a bioagent whose existence is known (such as the well known bacterial species Staphylococcus aureus for example) but which is not known to be in a sample to be analyzed and/or (iii) a bioagent that is known or suspected of being present in a sample but whose sub-species characteristics are not known (such as a bacterial resistance genotype like the QRDR region of Staphyoicoccus aureus species). For example, if the method for identification of coronaviruses disclosed in commonly owned U.S. Pre-Grant Publication No. US2005-0266397 (incorporated herein by reference in its entirety) was to be employed prior to April 2003 to identify the SARS coronavirus in a clinical sample, both meanings of “unknown” bioagent are applicable since the SARS coronavirus was unknown to science prior to April, 2003 and since it was not known what bioagent (in this case a coronavirus) was present in the sample. On the other hand, if the method of U.S. Pre-Grant Publication No. US2005-0266397 was to be employed subsequent to April 2003 to identify the SARS coronavirus in a clinical sample, only the second meaning (ii) of “unknown” bioagent would apply because the SARS coronavirus became known to science subsequent to April 2003 but because it was not known what bioagent was present in the sample.

As used herein, the term “molecular mass” refers to the mass of a compound as determined using mass spectrometry. Herein, the compound is preferably a nucleic acid, more preferably a double stranded nucleic acid, still more preferably a double stranded DNA nucleic acid and is most preferably an amplicon. When the nucleic acid is double stranded the molecular mass is determined for both strands. Here, the strands are separated either before introduction into the mass spectrometer, or the strands are separated by the mass spectrometer (for example, electro-spray ionization will separate the hybridized strands). The molecular mass of each strand is measured by the mass spectrometer.

As used herein, the term “base composition” refers to the number of each residue comprising an amplicon, without consideration for the linear arrangement of these residues in the strand(s) of the amplicon. The amplicon residues comprise, adenosine (A), guanosine (G), cytidine,

(c), (deoxy)thymidine (T), uracil (U), inosine (I), nitroindoles such as 5-nitroindole or 3-nitropyrrole, dP or dK (Hill et al.), an acyclic nucleoside analog containing 5-nitroindazole (Van Aerschot et al., Nucleosides and Nucleotides, 1995, 14, 1053-1056), the purine analog 1-(2-deoxy-.beta.-D-ribofuranosyl)-imidazole-4-carboxamide, 2,6-diaminopurine, 5-propynyluracil, 5-propynylcytosine, phenoxazines, including G-clamp, 5-propynyl deoxy-cytidine, deoxy-thymidine nucleotides, 5-propynylcytidine, 5-propynyluridine and mass tag modified versions thereof, including 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises 15.sup.N or 13.sup.C or both 15.sup.N and 13.sup.C. Preferably, the non-natural nucleosides used herein include 5-propynyluracil, 5-propynylcytosine and inosine. Herein the base composition for an unmodified DNA amplicon is notated as A.sub.wG.sub.xC.sub.yT.sub.z, wherein w, x, y and z are each independently a whole number representing the number of said nucleoside residues in an amplicon. Base compositions for amplicons comprising modified nucleosides are similarly notated to indicate the number of said natural and modified nucleosides in an amplicon. Base compositions are calculated from a molecular mass measurement of an amplicon, as described below. The calculated base composition for any given amplicon is then compared to a database of base compositions. A match between the calculated base composition and a single database entry reveals the identity of the bioagent.

As is used herein, the term “base composition signature” refers to the base composition generated by any one particular amplicon. The base composition signature for each of one or more amplicons provides a fingerprint for identifying the bioagent(s) present in a sample. Base composition signatures are unique for each genotype of the bioagent.

As used herein, the term “database” is used to refer to a collection of base composition and/or molecular mass data. The base composition and/or molecular mass data in the database is indexed to bioagents and to primer pairs. The base composition data reported in the database comprises the number of each nucleoside in an amplicon that would be generated for each bioagent using each primer pair. The database can be populated by empirical data. In this aspect of populating the database, a bioagent is selected and a primer pair is used to generate an amplicon. The amplicon's molecular mass is determined using a mass spectrometer and the base composition calculated therefrom. An entry in the database is made to associate the base composition and/or molecular mass with the bioagent and the primer pair used. The database may also be populated using other databases comprising bioagent information. For example, using the GenBank database it is possible to perform electronic PCR using an electronic representation of a primer pair. This in silico method will provide the base composition for any or all selected bioagent(s) stored in the GenBank database. The information is then used to populate the base composition database as described above. A base composition database can be in silico, a written table, a reference book, a spreadsheet or any form generally amenable to databases. Preferably, it is in silico. The database can similarly be populated with molecular masses that is gathered either empirically or is calculated from other sources such as GenBank.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” “residue,” or deoxynucleotide triphosphate (dNTP). As is used herein, a nucleobase includes natural and modified residues, as described herein.

As used herein, a “wobble base” is a variation in a codon found at the third nucleotide position of a DNA triplet. Variations in conserved regions of sequence are often found at the third nucleotide position due to redundancy in the amino acid code.

As used herein, “housekeeping gene” refers to a gene encoding a protein or RNA involved in basic functions required for survival and reproduction of a bioagent. Housekeeping genes include, but are not limited to, genes encoding RNA or proteins involved in translation, replication, recombination and repair, transcription, nucleotide metabolism, amino acid metabolism, lipid metabolism, energy generation, uptake, secretion and the like. In some embodiments, the primers are configured to produce amplicons from within a housekeeping gene.

As used herein, a “bioagent division” is defined as group of bioagents above the species level and includes but is not limited to, orders, families, genus, classes, clades, genera or other such groupings of bioagents above the species level.

As used herein, a “sub-species characteristic” is a genetic characteristic that provides the means to distinguish two members of the same bioagent species. For example, one bacterial strain could be distinguished from another bacterial strain of the same species by possessing a genetic change (e.g., for example, a nucleotide deletion, addition or substitution) in one of the bacterial genes, such as the GyrA gene.

As used herein, “triangulation identification” means the employment of more than one primer pair to generate a corresponding amplicon for identification of a bioagent. The more than one primer pair can be used in individual wells or in a multiplex PCR assay. Alternatively, PCR reaction may be carried out in single wells comprising a different primer pair in each well. Following amplification, the amplicons are pooled into a single well or container which is then subjected to molecular mass analysis. The combination of pooled amplicons can be chosen such that the expected ranges of molecular masses of individual amplicons are not overlapping and thus will not complicate identification of signals. Triangulation works as a process of elimination, wherein a first primer pair identifies that an unknown bioagent may be one of a group of bioagents. Subsequent primer pairs are used in triangulation identification to further refine the identity of the bioagent amongst the subset of possibilities generated with the earlier primer pair. Triangulation identification is complete when the identity of the bioagent is determined. The triangulation identification process is also used to reduce false negative and false positive signals, and enable reconstruction of the origin of hybrid or otherwise engineered bioagents. For example, identification of the three part toxin genes typical of B. anthracis (Bowen et al., J. Appl. Microbiol., 1999, 87, 270-278) in the absence of the expected signatures from the B. anthracis genome would suggest a genetic engineering event.

As is used herein, the term “single primer pair identification” means that one or more bioagents can be identified using a single primer pair. A base composition signature for an amplicon may singly identify one or more bioagents.

As used herein, the term “etiology” refers to the causes or origins, of diseases or abnormal physiological conditions.

As used herein, “population genotype” refers to the one or more genotypes for a particular gene, locus, or nucleotide position that are present in a population of bioagents. In some embodiments, the population comprises a plurality of bioagents, all with a single genotype for a particular gene, locus or nucleotide position. In these embodiments, the population genotype comprises one genotype for that gene locus or position. In other embodiments, the population of bioagents is a “mixed population,” in which the plurality of bioagents has at least two distinct genotypes for a particular gene, locus or nucleotide position. In this embodiment, the population genotype comprises at least two distinct genotypes for that gene, locus or position.

The term “sample” in the present specification and claims is used in its broadest sense. On the one hand it is meant to include a specimen or culture (e.g., microbiological cultures). Preferably, the sample is from a human patient suspected of having a bacterial infection, for example, a blood, tissue, or wound sample. More preferably it is a blood, tissue, or wound swab. On the other hand, it is meant to include both biological and environmental samples. A sample may include a specimen of synthetic origin. Biological samples may be from an animal, including human, and may be fluid, solid (e.g., stool) or tissue, as well as liquid or solid food and feed products or ingredients such as dairy items, vegetables, meat and meat by-products, or waste. Biological samples may be obtained from all of the various families of domestic animals, as well as feral or wild animals, including, but not limited to, such animals as ungulates, bear, fish, lagamorphs, rodents, etc. Environmental samples include environmental material such as surface matter, soil, water, air and industrial samples, as well as samples obtained from food and dairy processing instruments, apparatus, equipment, utensils, disposable and non-disposable items. These examples are not to be construed as limiting the sample types applicable to the present invention. The term “source of target nucleic acid” refers to any sample that contains nucleic acids (RNA or DNA). Particularly preferred sources of target nucleic acids are biological samples including, but not limited to blood, saliva, cerebral spinal fluid, pleural fluid, milk, lymph, sputum and semen. In some embodiments, the sample is purified. The term “sample source” refers to the source of the sample, for example, the animal, human, fluid, tissue, culture, or other source from which the sample was isolated and/or purified.

Provided herein are methods for detection and identification of bioagents in an unbiased manner using bioagent identifying amplicons. In one aspect, the methods are for detection and identification of population genotype for a population of bioagents. Primers are selected to hybridize to conserved sequence regions of nucleic acids derived from a bioagent and which bracket (flank) variable sequence regions to yield a bioagent identifying amplicon which can be amplified and which is amenable to molecular mass determination. The molecular mass is converted to a base composition, which indicates the number of each nucleotide in the amplicon. The molecular mass or corresponding base composition signature of the amplicon is then queried against a database of molecular masses or base composition signatures indexed to bioagents and to the primer pair used to generate the amplicon. A match of the measured base composition to a database entry base composition associates the sample bioagent to an indexed bioagent in the database. Thus, the identity of the unknown bioagent or population of bioagents is determined. Prior knowledge of the unknown bioagent or population of bioagents is not necessary. In some instances, the measured base composition associates with more than one database entry base composition. Thus, a second/subsequent primer pair is used to generate an amplicon, and its measured base composition is similarly compared to the database to determine its identity in triangulation identification. For example, a first primer pair might identify that a bacterial bioagent is present in a sample that is a member of the Staphylococcus genus. A second primer might determine that it is a member of the Staphylococcus aureus species. A third primer pair might identify that the bioagent is resistant to quinolones. Furthermore, the method can be applied to rapid parallel multiplex analyses, the results of which can be employed in a triangulation identification strategy. The present method provides rapid throughput and does not require nucleic acid sequencing of the amplified target sequence for bioagent detection and identification.

In some embodiments, the methods are performed on nucleic acids comprised in a sample suspected of comprising a population of bioagents. In one aspect, the methods further comprise administering or delivering to the sample source an antibiotic regimen tailored to treat the identified genotypes for the population of bacteria. In this aspect, the antibiotic regimen is determined based on the genotype(s) identified by the method, with the goal of being able to effectively reduce the bioagents in the population. In one embodiment, the steps of the method are repeated “periodically” or more than one additional time following the initial identification. In one aspect, the periodic repeating of the steps is done at regular intervals. In other aspects, it is done sporadically or at irregular time points. In another aspect, it is done in response to a trigger, such as the appearance of one or more symptoms. In one aspect, the antibiotic regimen is modified based on one or more genotypes identified during the periodic repeating of the steps. In one embodiment, the antibiotic regimen comprises an antibiotic for treating quinolone resistant bacteria. In another embodiment, the antibiotic regimen comprises an antibiotic for treating quinolone sensitive bacteria. In one aspect, the antibiotic for treating quinolone sensitive bacteria is a quinolone. In one aspect, it is a fluoroquinolone.

Despite enormous biological diversity, all forms of life on earth share sets of essential, common features in their genomes. Since genetic data provide the underlying basis for identification of bioagents by the current methods, it is necessary to select segments of nucleic acids which ideally provide enough variability to distinguish each individual bioagent and whose molecular mass is amenable to molecular mass determination.

In some embodiments, at least one bacterial nucleic acid segment is amplified in the process of identifying the bioagent. Thus, the nucleic acid segments that can be amplified by the primers disclosed herein and that provide enough variability to distinguish each individual bioagent and whose molecular masses are amenable to molecular mass determination are herein described as bioagent identifying amplicons.

In some embodiments, bioagent identifying amplicons amenable to molecular mass determination that are produced by the primers described herein are either of a length, size and/or mass compatible with the particular mode of molecular mass determination or compatible with a means of providing a predictable fragmentation pattern in order to obtain predictable fragments of a length compatible with the particular mode of molecular mass determination. Such means of providing a predictable fragmentation pattern of an amplicon include, but are not limited to, cleavage with restriction enzymes or cleavage primers, for example. Thus, in some embodiments, bioagent identifying amplicons are larger than 200 nucleobases and are amenable to molecular mass determination following restriction digestion. Methods of using restriction enzymes and cleavage primers are well known to those with ordinary skill in the art.

In some embodiments, amplicons corresponding to bioagent identifying amplicons are obtained using the polymerase chain reaction (PCR) which is a routine method to those with ordinary skill in the molecular biology arts. Other amplification methods may be used such as ligase chain reaction (LCR), low-stringency single primer PCR, and multiple strand displacement amplification (MDA). These methods are also known to those with ordinary skill. (Michael, S F., Biotechniques (1994), 16:411-412 and Dean et al., Proc. Natl. Acad. Sci. U.S.A. (2002), 99, 5261-5266)

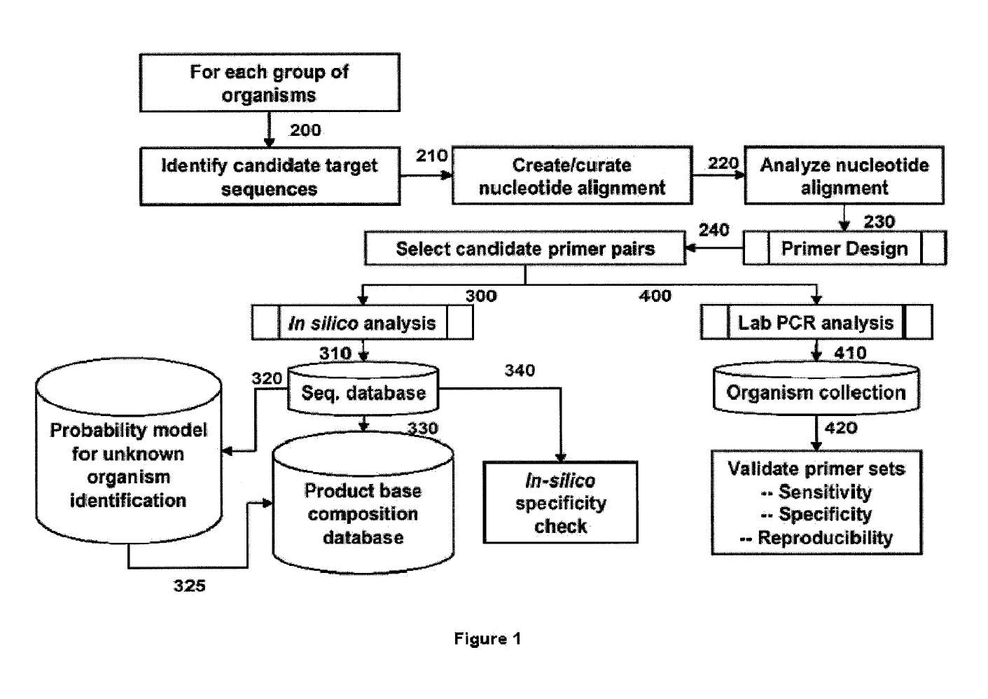

A representative process flow diagram used for primer selection and validation process is outlined in FIG. 1. For each group of diverse organisms, candidate target sequences are identified (200) from which nucleotide alignments are created (210) and analyzed (220). Primers are then configured by selecting appropriate priming regions (230) to facilitate the selection of candidate primer pairs (240). The primer pair sequence is a “best fit” amongst the aligned sequences, meaning that the primer pair sequence may or may not be fully complementary to the hybridization region on any one of the bioagents in the alignment. Thus, bets fit primer pair sequences are those with sufficient complementarity with two or more bioagents to hybridize with the two or more bioagents and generate an amplicon. The primer pairs are then subjected to in silico analysis by electronic PCR (ePCR) (300) wherein bioagent identifying amplicons are obtained from sequence databases such as GenBank or other sequence collections (310) and checked for specificity in silico (320). Bioagent identifying amplicons obtained from ePCR of GenBank sequences (310) can also be analyzed by a probability model which predicts the capability of a given amplicon to identify unknown bioagents. Preferably, the base compositions of amplicons with favorable probability scores are then stored in a base composition database (325). Alternatively, base compositions of the bioagent identifying amplicons obtained from the primers and GenBank sequences can be directly entered into the base composition database (330). Candidate primer pairs (240) are validated by in vitro amplification by a method such as PCR analysis (400) of nucleic acid from a collection of organisms (410). Amplicons thus obtained are analyzed to confirm the sensitivity, specificity and reproducibility of the primers used to obtain the amplicons (420).

Synthesis of primers is well known and routine in the art. The primers may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

The primers are employed as compositions for use in methods for identification of bacterial bioagents as follows: a primer pair composition is contacted with nucleic acid (such as, for example, DNA or DNA reverse transcribed from RNA) of an unknown bacterial bioagent. The nucleic acid is then amplified by a nucleic acid amplification technique, such as PCR for example, to obtain an amplicon that represents a bioagent identifying amplicon. The molecular mass of each strand of the double-stranded amplicon is determined by a molecular mass measurement technique such as mass spectrometry for example. Preferably the two strands of the double-stranded amplicon are separated during the ionization process; however, they may be separated prior to mass spectrometry measurement. In some embodiments, the mass spectrometer is electrospray Fourier transform ion cyclotron resonance mass spectrometry (ESI-FTICR-MS) or electrospray time of flight mass spectrometry (ESI-TOF-MS). A list of possible base compositions can be generated for the molecular mass value obtained for each strand and the choice of the correct base composition from the list is facilitated by matching the base composition of one strand with a complementary base composition of the other strand. The measured molecular mass or base composition calculated therefrom is then compared with or querried against a database of molecular masses or base compositions indexed to primer pairs and to known bacterial bioagents. A match between the measured molecular mass or base composition of the amplicon and the database molecular mass or base composition for that indexed primer pair will associate the measured molecular mass or base composition with an indexed bacterial bioagent, thus indicating the identity of the unknown bioagent. In some embodiments, the primer pair used is one of the primer pairs of Table 1. In some embodiments, the method is repeated using a different primer pair to resolve possible ambiguities in the identification process or to improve the confidence level for the identification assignment (triangulation identification).

In some embodiments, a bioagent identifying amplicon may be produced using only a single primer (either the forward or reverse primer of any given primer pair), provided an appropriate amplification method is chosen, such as, for example, low stringency single primer PCR (LSSP-PCR). Adaptation of this amplification method in order to produce bioagent identifying amplicons can be accomplished by one with ordinary skill in the art without undue experimentation. (Pena, S D J et al., Proc. Natl. Acad. Sci. U.S.A (1994) 91, 1946-1949).

In some embodiments, the oligonucleotide primers are broad range survey primers which hybridize to conserved regions of a nucleic acid encoding a gene that is common to all known members of the Staphylococcus genus, though the sequences of the gene that are within the variable region vary. The broad range primer may identify the unknown bioagent, depending on which bioagent is in the sample. In other cases, the molecular mass or base composition of an amplicon does not provide enough resolution to unambiguously identify the unknown bioagent as any one bacterial bioagent at or below the species level. These cases benefit from further analysis of one or more an amplicons generated from at least one additional broad range survey primer pair or from at least one additional division-wide primer pair or from at least one additional drill-down primer pair. Identification of sub-species characteristics is often critical for determining proper clinical treatment of viral infections, or in rapidly responding to an outbreak of a new viral strain to prevent massive epidemic or pandemic.

In some embodiments, the primers used for amplification hybridize to and amplify genomic DNA, DNA of bacterial plasmids, transposons and other exogenous nucleic acid, or DNA reverse transcribed from RNA. Among other things, the identification of non-bacterial nucleic acids or combinations of bacterial and non-bacterial nucleic acids are useful for detecting bioengineered bioagents.

In some embodiments, the primers used for amplification hybridize directly to bacterial RNA and act as reverse transcription primers for obtaining DNA from direct amplification of bacterial RNA. Methods of amplifying RNA to produce cDNA using reverse transcriptase are well known to those with ordinary skill in the art and can be routinely established without undue experimentation.

One with ordinary skill in the art of design of amplification primers will recognize that a given primer need not hybridize with 100% complementarity in order to effectively prime the synthesis of a complementary nucleic acid strand in an amplification reaction. Primer pair sequences may be a “best fit” amongst the aligned bioagent sequences, thus not be fully complementary to the hybridization region on any one of the bioagents in the alignment. Moreover, a primer may hybridize over one or more segments such that intervening or adjacent segments are not involved in the hybridization event. (e.g., for example, a loop structure or a hairpin structure). The primers may comprise at least 70%, at least 75%, at least 80%, at least 85%, at least 90%, at least 95% or at least 99% sequence identity with any of the primers listed in Table 1 or other primer disclosed herein. Thus, in some embodiments, an extent of variation of 70% to 100%, or any range falling within, of the sequence identity is possible relative to the specific primer sequences disclosed herein. Determination of sequence identity is described in the following example: a primer 20 nucleobases in length which is identical to another 20 nucleobase primer having two non-identical residues has 18 of 20 identical residues (18/20=0.9 or 90% sequence identity). In another example, a primer 15 nucleobases in length having all residues identical to a 15 nucleobase segment of primer 20 nucleobases in length would have 15/20=0.75 or 75% sequence identity with the 20 nucleobase primer. Percent identity need not be a whole number, for example when a 28 consecutive nucleobase primer is completely identical to a 31 consecutive nucleobase primer (28/31=0.9032 or 90.3% identical). Similarly, either or both of the primers of the primer pairs provided herein may comprise 0-9 nucleobase deletions, additions, and/or substitutions relative to any of the primers listed in Table 1, or elsewhere herein. In other words, either or both of the primers may comprise 0, 1, 2, 3, 4, 5, 6, 7, 8 or 9 nucleobase deletions, 0, 1, 2, 3, 4, 5, 6, 7, 8 or 9 nucleobase additions, 0, 1, 2, 3, 4, 5, 6, 7, 8 or 9 nucleobase substitutions relative to the sequences of any of the primers disclosed herein. In one aspect, the primers comprise the sequence of any of the primers listed in Table 1 with the non-templated T residue removed from the 5′ terminus. In one aspect, the primers comprise the sequence of any of the primers listed in Table 1 with the non-templated T residue removed from the 5′ terminus and comprising 0-9 nucleobase deletions, additions, and/or substitutions.

Percent homology, sequence identity or target complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, target complementarity of primers with respect to the conserved priming regions of bacterial nucleic acid, is between about 70% and about 80%. In other embodiments, homology, sequence identity or complementarity, is between about 80% and about 90%. In yet other embodiments, homology, sequence identity or complementarity, is at least 90%, at least 92%, at least 94%, at least 95%, at least 96%, at least 97%, at least 98%, at least 99% or is 100%.

In some embodiments, the primers described herein comprise at least 70%, at least 75%, at least 80%, at least 85%, at least 90%, at least 92%, at least 94%, at least 95%, at least 96%, at least 98%, or at least 99%, or 100% (or any range falling within) sequence identity with the primer sequences specifically disclosed herein.

One with ordinary skill is able to calculate percent sequence identity or percent sequence homology and is able to determine, without undue experimentation, the effects of variation of primer sequence identity on the function of the primer in its role in priming synthesis of a complementary strand of nucleic acid for production of a corresponding bioagent identifying amplicon.

In some embodiments, the oligonucleotide primers are 13 to 35 nucleobases in length (13 to 35 linked nucleotide residues). These embodiments comprise oligonucleotide primers 13, 14, 15, 16, 17, 18, 19, 20, 21, 22, 23, 24, 25, 26, 27, 28, 29, 30, 31, 32, 33, 34 or 35 nucleobases in length, or any range therewithin.

In some embodiments, any given primer comprises a modification comprising the addition of a non-templated T residue to the 5′ end of the primer (i.e., the added T residue does not necessarily hybridize to the nucleic acid being amplified). The addition of a non-templated T residue has an effect of minimizing the addition of non-templated A residues as a result of the non-specific enzyme activity of Taq polymerase (Magnuson et al., Biotechniques, 1996, 21, 700-709), an occurrence which may lead to ambiguous results arising from molecular mass analysis. Primer pairs comprising the sequence of any of the primer pairs described herein, but lacking the non-templated T residue at the 5′ end of the primer are also encompassed by this disclosure.

Primers may contain one or more universal bases. Because any variation (due to codon wobble in the third position) in the conserved regions among species is likely to occur in the third position of a DNA (or RNA) triplet, oligonucleotide primers can be configured such that the nucleotide corresponding to this position is a base which can bind to more than one nucleotide, referred to herein as a “universal nucleobase.” For example, under this “wobble” pairing, inosine (I) binds to U, C or A; guanine (G) binds to U or C, and uridine (U) binds to U or C. Other examples of universal nucleobases include nitroindoles such as 5-nitroindole or 3-nitropyrrole (Loakes et al., Nucleosides and Nucleotides, 1995, 14, 1001-1003), the degenerate nucleotides dP or dK (Hill et al.), an acyclic nucleoside analog containing 5-nitroindazole (Van Aerschot et al., Nucleosides and Nucleotides, 1995, 14, 1053-1056) or the purine analog 1-(2-deoxy-.beta.-D-ribofuranosyl)-imidazole-4-carboxamide (Sala et al., Nucl. Acids Res., 1996, 24, 3302-3306).

In some embodiments, to compensate for the somewhat weaker binding by the wobble base, the oligonucleotide primers are configured such that the first and second positions of each triplet are occupied by nucleotide analogs which bind with greater affinity than the unmodified nucleotide. Examples of these analogs include, but are not limited to, 2,6-diaminopurine which binds to thymine, 5-propynyluracil which binds to adenine and 5-propynylcytosine and phenoxazines, including G-clamp, which binds to G. Propynylated pyrimidines are described in U.S. Pat. Nos. 5,645,985, 5,830,653 and 5,484,908, each of which is commonly owned and incorporated herein by reference in its entirety. Propynylated primers are described in U.S. Pre-Grant Publication No. 2003-0170682; also commonly owned and incorporated herein by reference in its entirety. Phenoxazines are described in U.S. Pat. Nos. 5,502,177, 5,763,588, and 6,005,096, each of which is incorporated herein by reference in its entirety. G-clamps are described in U.S. Pat. Nos. 6,007,992 and 6,028,183, each of which is incorporated herein by reference in its entirety.

In some embodiments, to enable broad priming of rapidly evolving bioagents, primer hybridization is enhanced using primers and probes containing 5-propynyl deoxy-cytidine and deoxy-thymidine nucleotides. These modified primers offer increased affinity and base pairing selectivity.

In some embodiments, non-template primer tags are used to increase the melting temperature (T.sub.m) of a primer-template duplex in order to improve amplification efficiency. A non-template tag is at least three consecutive A or T nucleotide residues on a primer which are not complementary to the template. In any given non-template tag, A can be replaced by C or G and T can also be replaced by C or G. Although Watson-Crick hybridization is not expected to occur for a non-template tag relative to the template, the extra hydrogen bond in a G-C pair relative to an A-T pair confers increased stability of the primer-template duplex and improves amplification efficiency for subsequent cycles of amplification when the primers hybridize to strands synthesized in previous cycles.

In other embodiments, propynylated tags may be used in a manner similar to that of the non-template tag, wherein two or more 5-propynylcytidine or 5-propynyluridine residues replace template matching residues on a primer. In other embodiments, a primer contains a modified internucleoside linkage such as a phosphorothioate linkage, for example.

In some embodiments, the primers contain mass-modifying tags. Reducing the total number of possible base compositions of a nucleic acid of specific molecular weight provides a means of avoiding a persistent source of ambiguity in determination of base composition of amplicons. Addition of mass-modifying tags to certain nucleobases of a given primer will result in simplification of de novo determination of base composition of a given bioagent identifying amplicon from its molecular mass.

In some embodiments, the mass modified nucleobase comprises one or more of the following: for example, 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises .sup.15N or .sup.13C or both .sup.15N and .sup.13C.

In some embodiments, the molecular mass of a given bioagent identifying amplicon is determined by mass spectrometry. Mass spectrometry has several advantages, not the least of which is high bandwidth characterized by the ability to separate (and isolate) many molecular peaks across a broad range of mass to charge ratio (m/z). Thus mass spectrometry is intrinsically a parallel detection scheme without the need for radioactive or fluorescent labels since every amplicon is identified by its molecular mass. The current state of the art in mass spectrometry is such that less than femtomole quantities of material can be readily analyzed to afford information about the molecular contents of the sample. An accurate assessment of the molecular mass of the material can be quickly obtained, irrespective of whether the molecular weight of the sample is several hundred, or in excess of one hundred thousand atomic mass units (amu) or Daltons.

In some embodiments, intact molecular ions are generated from amplicons using one of a variety of ionization techniques to convert the sample to gas phase. These ionization methods include, but are not limited to, electrospray ionization (ES), matrix-assisted laser desorption ionization (MALDI) and fast atom bombardment (FAB). Upon ionization, several peaks are observed from one sample due to the formation of ions with different charges. Averaging the multiple readings of molecular mass obtained from a single mass spectrum affords an estimate of molecular mass of the bioagent identifying amplicon. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

The mass detectors used include, but are not limited to, Fourier transform ion cyclotron resonance mass spectrometry (FT-ICR-MS), time of flight (TOF), ion trap, quadrupole, magnetic sector, Q-TOF, and triple quadrupole.

In some embodiments, assignment of previously unobserved base compositions (also known as “true unknown base compositions”) to a given phylogeny can be accomplished via the use of pattern classifier model algorithms. Base compositions, like sequences, vary slightly from strain to strain within species, for example. In some embodiments, the pattern classifier model is the mutational probability model. On other embodiments, the pattern classifier is the polytope model.

In one embodiment, it is possible to manage this diversity by building “base composition probability clouds” around the composition constraints for each species. This permits identification of organisms in a fashion similar to sequence analysis. Using three primer pairs, a “pseudo four-dimensional plot” can be used to visualize the concept of base composition probability clouds. Optimal primer design requires optimal choice of bioagent identifying amplicons and maximizes the separation between the base composition signatures of individual bioagents. Areas where clouds overlap indicate regions that may result in a misclassification, a problem which is overcome by a triangulation identification process using bioagent identifying amplicons not affected by overlap of base composition probability clouds.

In some embodiments, base composition probability clouds provide the means for screening potential primer pairs in order to avoid potential misclassifications of base compositions. In other embodiments, base composition probability clouds provide the means for predicting the identity of an unknown bioagent whose assigned base composition was not previously observed and/or indexed in a bioagent identifying amplicon base composition database due to evolutionary transitions in its nucleic acid sequence. Thus, in contrast to probe-based techniques, mass spectrometry determination of base composition does not require prior knowledge of the composition or sequence in order to make the measurement.

Provided herein are bioagent classifying information at a level sufficient to identify a given bioagent. Furthermore, the process of determining a previously unknown base composition for a given bioagent (for example, in a case where sequence information is unavailable) has downstream utility by providing additional bioagent indexing information with which to populate base composition databases. The process of future bioagent identification is thus greatly improved as more base composition signature indexes become available in base composition databases.

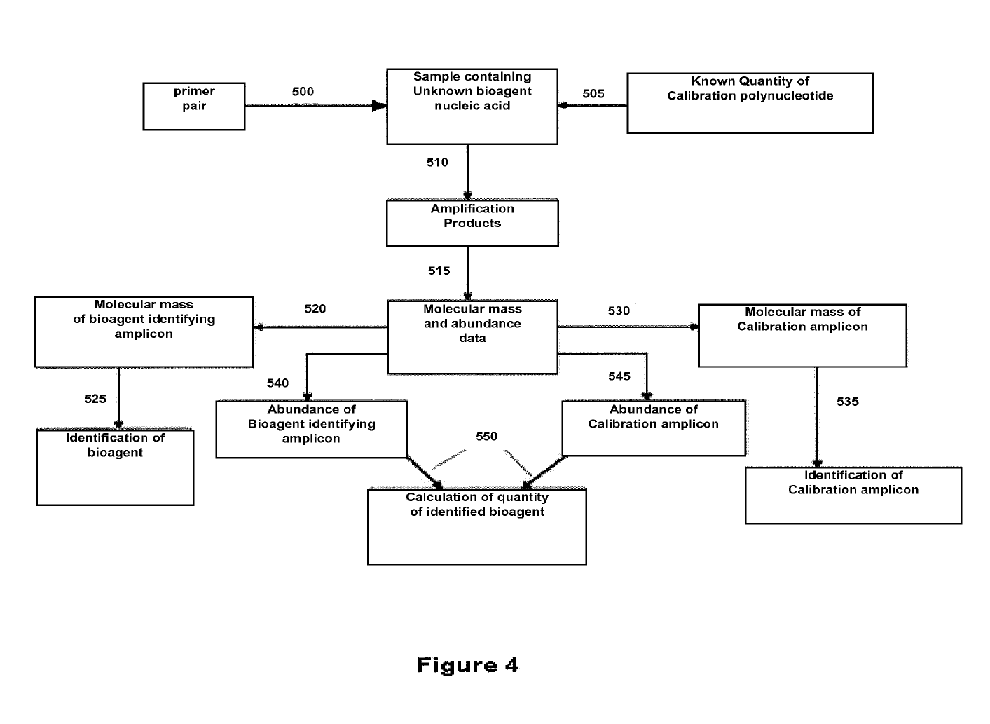

In some embodiments, the identity and quantity of an unknown bioagent can be determined using the process illustrated in FIG. 4. Primers (500) and a known quantity of a calibration polynucleotide (505) is added to a sample containing nucleic acid of an unknown bioagent. The total nucleic acid in the sample is then subjected to an amplification reaction (510) to obtain amplicons. The molecular masses of amplicons are determined (515) from which are obtained molecular mass and abundance data. The molecular mass of the bioagent identifying amplicon (520) provides for its identification (525) and the molecular mass of the calibration amplicon obtained from the calibration polynucleotide (530) provides for its quantification (535). The abundance data of the bioagent identifying amplicon is recorded (540) and the abundance data for the calibration data is recorded (545), both of which are used in a calculation (550) which determines the quantity of unknown bioagent in the sample.

A sample comprising an unknown bioagent is contacted with a primer pair which amplifies the nucleic acid from the bioagent, and a known quantity of a polynucleotide that comprises a calibration sequence. The rate of amplification is reasonably assumed to be similar for the nucleic acid of the bioagent and for the calibration sequence. The amplification reaction then produces two amplicons: a bioagent identifying amplicon and a calibration amplicon. The bioagent identifying amplicon and the calibration amplicon should be distinguishable by molecular mass while being amplified at essentially the same rate. Effecting differential molecular masses can be accomplished by choosing as a calibration sequence, a representative bioagent identifying amplicon (from a specific species of bioagent) and performing, for example, a 2-8 nucleobase deletion or insertion within the variable region between the two priming sites. The amplified sample containing the bioagent identifying amplicon and the calibration amplicon is then subjected to molecular mass analysis by mass spectrometry, for example. The resulting molecular mass analysis of the nucleic acid of the bioagent and of the calibration sequence provides molecular mass data and abundance data for the nucleic acid of the bioagent and of the calibration sequence. The molecular mass data obtained for the nucleic acid of the bioagent enables identification of the unknown bioagent by base composition analysis. The abundance data enables calculation of the quantity of the bioagent, based on the knowledge of the quantity of calibration polynucleotide contacted with the sample.

In some embodiments, construction of a standard curve where the amount of calibration polynucleotide spiked into the sample is varied provides additional resolution and improved confidence for the determination of the quantity of bioagent in the sample. The use of standard curves for analytical determination of molecular quantities is well known to one with ordinary skill and can be performed without undue experimentation. Alternatively, the calibration polynucleotide can be amplified in into own reaction well or wells under the same conditions as the bioagent. A standard curve can be prepared therefrom, and a relative abundance of the bioagent determined by methods such as linear regression. In some embodiments, multiplex amplification is performed where multiple bioagent identifying amplicons are amplified with multiple primer pairs which also amplify the corresponding standard calibration sequences. In this or other embodiments, the standard calibration sequences are optionally included within a single construct (preferably a vector) which functions as the calibration polynucleotide. Competitive PCR, quantitative PCR, quantitative competitive PCR, multiplex and calibration polynucleotides are all methods and materials well known to those ordinarily skilled in the art and can be performed without undue experimentation.

In some embodiments, the calibrant polynucleotide is used as an internal positive control to confirm that amplification conditions and subsequent analysis steps are successful in producing a measurable amplicon. Even in the absence of copies of the genome of a bioagent, the calibration polynucleotide should give rise to a calibration amplicon. Failure to produce a measurable calibration amplicon indicates a failure of amplification or subsequent analysis step such as amplicon purification or molecular mass determination. Reaching a conclusion that such failures have occurred is in itself, a useful event. In some embodiments, the calibration sequence is comprised of DNA. In some embodiments, the calibration sequence is comprised of RNA.

In the preferred embodiment, the calibration sequence is inserted into a vector which then itself functions as the calibration polynucleotide. In some embodiments, more than one calibration sequence is inserted into the vector that functions as the calibration polynucleotide. Such a calibration polynucleotide is herein termed a “combination calibration polynucleotide.” The process of inserting polynucleotides into vectors is routine to those skilled in the art and can be accomplished without undue experimentation. Thus, it should be recognized that the calibration method should not be limited to the embodiments described herein. The calibration method can be applied for determination of the quantity of any bioagent identifying amplicon when an appropriate standard calibrant polynucleotide sequence is configured and used. The process of choosing an appropriate vector for insertion of a calibrant is also a routine operation that can be accomplished by one with ordinary skill without undue experimentation.

It is preferable for some primer pairs to produce bioagent identifying amplicons within more conserved regions of Staphylococci bacteria while others produce bioagent identifying amplicons within regions that are likely to evolve more quickly. Primer pairs that characterize amplicons in a conserved region with low probability that the region will evolve past the point of primer recognition are useful as a broad range survey-type primer. Primer pairs that characterize an amplicon corresponding to an evolving genomic region are useful for distinguishing emerging strain variants.

The primer pairs described herein establish a platform for identifying members of the Staphylococcus genus. Base composition analysis eliminates the need for prior knowledge of bioagent sequence to generate hybridization probes. Thus, in another embodiment, there is provided a method for determining the etiology of a bacterial infection when the process of identification of bacteria is carried out in a clinical setting and, even when the bacteria is a new species never observed before. This is possible because the methods are not confounded by naturally occurring evolutionary variations (a major concern when using probe based or sequencing dependent methods for characterizing viruses that evolve rapidly). Measurement of molecular mass and determination of base composition is accomplished in an unbiased manner without sequence prejudice and without the need for specificity as is required with probes.

Another embodiment provides a means of tracking the spread of any species or strain of bacteria when a plurality of samples obtained from different locations are analyzed by the methods described above in an epidemiological setting. For example, a plurality of samples from a plurality of different locations is analyzed with primers which produce bioagent identifying amplicons, a subset of which contains a specific bacteria. The corresponding locations of the members of the bacteria-containing subset indicate the spread of the specific bacteria to the corresponding locations.

Also provided are kits for carrying out the methods described herein. In some embodiments, the kit may comprise a sufficient quantity of one or more primer pairs to perform an amplification reaction on a target polynucleotide from a bioagent to form a bioagent identifying amplicon. In some embodiments, the kit may comprise from one to fifty primer pairs, from one to twenty primer pairs, from one to ten primer pairs, from one to eight primer pairs or from two to five primer pairs. In some embodiments, the kit may comprise one or more primer pairs recited in Table 1. In a preferred embodiment, the kit comprises eight primer pairs from Table 1. In a preferred aspect the eight primer pairs comprised in the kit are selected from: SEQ ID NO.: 58:SEQ ID NO.:142, SEQ ID NO.: 62:SEQ ID NO.:147, SEQ ID NO.: 294:SEQ ID NO.:295, SEQ ID NO.: 35:SEQ ID NO.:121, SEQ ID NO.: 39:SEQ ID NO.:125, SEQ ID NO.: 47:SEQ ID NO.:132, SEQ ID NO.: 55:SEQ ID NO.:139, SEQ ID NO.: 21:SEQ ID NO.:104, SEQ ID NO.: 22:SEQ ID NO.:106, SEQ ID NO.: 70:SEQ ID NO.:155, SEQ ID NO.: 329:SEQ ID NO.: 330, SEQ ID NO.: 331:SEQ ID NO.:332, SEQ ID NO.: 2:SEQ ID NO.:5, SEQ ID NO.: 3:SEQ ID NO.:6, SEQ ID NO.: 3:SEQ ID NO.:7, and SEQ ID NO.: 4:SEQ ID NO.:5. In another preferred aspect, the eight primer pairs comprised in the kit are selected from: SEQ ID NO.: 72:SEQ ID NO.:156, SEQ ID NO.: 79:SEQ ID NO.:166, SEQ ID NO.: 76:SEQ ID NO.:162, SEQ ID NO.: 83:SEQ ID NO.:170, SEQ ID NO.: 87:SEQ ID NO.:172, SEQ ID NO.: 90:SEQ ID NO.:177, SEQ ID NO.: 93:SEQ ID NO.:180, SEQ ID NO.: 94:SEQ ID NO.:181, SEQ ID NO.: 72:SEQ ID NO.:158, SEQ ID NO.: 2:SEQ ID NO.:5, SEQ ID NO.: 3:SEQ ID NO.:6, SEQ ID NO.: 3:SEQ ID NO.:7, and SEQ ID NO.: 4:SEQ ID NO.:5. In another preferred embodiment, the kit comprises nine oligonucleotide primer pairs. In a preferred aspect, the nine oligonucleotide primer pairs are SEQ ID NO.: 58:SEQ ID NO.:142, SEQ ID NO.: 62:SEQ ID NO.:147, SEQ ID NO.: 294:SEQ ID NO.:295, SEQ ID NO.: 35:SEQ ID NO.:121, SEQ ID NO.: 39:SEQ ID NO.:125, SEQ ID NO.: 47:SEQ ID NO.:132, SEQ ID NO.: 55:SEQ ID NO.:139, SEQ ID NO.: 21:SEQ ID NO.:104, SEQ ID NO.: 22:SEQ ID NO.:106, SEQ ID NO.: 70:SEQ ID NO.:155, and SEQ ID NO.: 3:SEQ ID NO.:7. In another preferred aspect, the nine oligonucleotide primers comprised in the kit are SEQ ID NO.: 72:SEQ ID NO.:156, SEQ ID NO.: 79:SEQ ID NO.:166, SEQ ID NO.: 76:SEQ ID NO.:162, SEQ ID NO.: 83:SEQ ID NO.:170, SEQ ID NO.: 87:SEQ ID NO.:172, SEQ ID NO.: 90:SEQ ID NO.:177, SEQ ID NO.: 93:SEQ ID NO.:180, SEQ ID NO.: 94:SEQ ID NO.:181, SEQ ID NO.: 72:SEQ ID NO.:158, and SEQ ID NO.: 3:SEQ ID NO.:7. In another preferred embodiment, the kit comprises 17 oligonucleotide primer pairs. Preferrably, the 17 oligonucleotide primer pairs comprised in the kit are SEQ ID NO.: 58:SEQ ID NO.:142, SEQ ID NO.: 62:SEQ ID NO.:147, SEQ ID NO.: 294:SEQ ID NO.:295, SEQ ID NO.: 35:SEQ ID NO.:121, SEQ ID NO.: 39:SEQ ID NO.:125, SEQ ID NO.: 47:SEQ ID NO.:132, SEQ ID NO.: 55:SEQ ID NO.:139, SEQ ID NO.: 21:SEQ ID NO.:104, SEQ ID NO.: 22:SEQ ID NO.:106, SEQ ID NO.: 70:SEQ ID NO.:155, SEQ ID NO.: 72:SEQ ID NO.:156, SEQ ID NO.: 79:SEQ ID NO.:166, SEQ ID NO.: 76:SEQ ID NO.:162, SEQ ID NO.: 83:SEQ ID NO.:170, SEQ ID NO.: 87:SEQ ID NO.:172, SEQ ID NO.: 90:SEQ ID NO.:177, SEQ ID NO.: 93:SEQ ID NO.:180, SEQ ID NO.: 94:SEQ ID NO.:181, SEQ ID NO.: 72:SEQ ID NO.:158, and SEQ ID NO.: 3:SEQ ID NO.:7.

In some embodiments, the kit may comprise one or more broad range survey primer(s), division wide primer(s), or drill-down primer(s), or any combination thereof A kit may be configured so as to comprise select primer pairs for identification of a particular bioagent. For example, a broad range survey primer kit may be used initially to identify an unknown bioagent as a member of the genus Staphyolococcus. Another example of a division-wide kit may be used to distinguish Staphylococcus aureus from Staphylococcus epidermidis, for example. A drill-down kit may be used, for example, to distinguish resistance and sensitivity of bacteria to one or more antibiotics. In some embodiments, the kit may contain standardized calibration polynucleotides for use as internal amplification calibrants.

In some embodiments, the kit may also comprise a sufficient quantity of reverse transcriptase (if an RNA is to be identified for example), a DNA polymerase, suitable nucleoside triphosphates (including any of those described above), a DNA ligase, and/or reaction buffer, or any combination thereof, for the amplification processes described above. A kit may further include instructions pertinent for the particular embodiment of the kit, such instructions describing the primer pairs and amplification conditions for operation of the method. A kit may also comprise amplification reaction containers such as microcentrifuge tubes and the like. A kit may also comprise reagents or other materials for isolating bioagent nucleic acid or bioagent identifying amplicons from amplification, including, for example, detergents, solvents, or ion exchange resins which may be linked to magnetic beads. A kit may also comprise a table of measured or calculated molecular masses and/or base compositions of bioagents using the primer pairs of the kit.

In one embodiment, population genotypes for mixed populations of bioagents can are identified. Population genotypes for mixed populations can be identified with high sensitivity by PCR-ESI/MS because amplified bioagent nucleic acids having different base compositions appear in different positions in the mass spectrum. The dynamic range for mixed PCR-ESI/MS detections has previously been determined to be approximately 100:1 (Hofstadler, S. A. et al., Inter. J. Mass Spectrom. (2005) 242, 23), which allows for detection of genotype variants with as low as 1% abundance in a mixed population. This detection using PCR-ESI/MS surveillance does not require secondary testing.

The following examples serve only as illustration, and not limitation.

### EXAMPLES

### Example 1

### Selection of Design and Validation of Primers that Define Bioagent Identifying Amplicons for Staphylococcus

For design of primers that define Staphylococcus identifying amplicons, a series of Staphylococcus genome segment sequences were obtained, aligned and scanned for regions where pairs of PCR primers would amplify products of about 45 to about 200 nucleotides in length and distinguish individual species, strains, and/or genotypes by their molecular masses or base compositions. A typical process shown in FIG. 1 is employed for this type of analysis.

A database of expected base compositions for each primer region was generated using an in silico PCR search algorithm, such as (ePCR). An existing RNA structure search algorithm (Macke et al., Nucl. Acids Res., 2001, 29, 4724-4735, which is incorporated herein by reference in its entirety) has been modified to include PCR parameters such as hybridization conditions, mismatches, and thermodynamic calculations (SantaLucia, Proc. Natl. Acad. Sci. U.S.A., 1998, 95, 1460-1465, which is incorporated herein by reference in its entirety). This structure search algorithm can be used for other nucleic acids, such as DNA. This also provides information on primer specificity of the selected primer pairs.

Table 1 lists a collection of primers (sorted by primer pair number) configured to identify Staphylococcus bioagents using the methods described herein. The primer pair number is an in-house database index number. Primer sites (conserved regions which primers were configured to hybridize within) were identified on Staphylococcus genes including arcC, aroE, ermA, ermC, gmk, gyrA, mecA, mecR1, mupR, nuc, pta, pvluk, tpi, tsst, tufB, and yqi. The forward and reverse primer names shown in Table 1 indicate the gene region of a bacterial genome to which the forward and reverse primers hybridize relative to a reference sequence. The forward primer name GYRA_NC002953-7005-9668_{—}234_{—}261_F indicates that the forward primer (“F”) hybridizes to the GyrA gene (“GYRA”), specifically to residues 234-261 (“234_{—}261”) of a reference sequence represented by a sequence extraction of coordinates 7005-9668 (SEQ ID NO.: 8) from GenBank gi number 49484912 (as indicated by cross-references in Table 2 for the prefix “GYRA_NC002953”). This sequence extraction reference includes sequence encoding for the gyrA gene (“GYRA”). The primer pair name codes appearing in Table 1 are defined in Table 2. For example, Table 2 lists gene abbreviations and GenBank gi numbers that correspond with each primer name code. For example, for the above-mentioned primer pair has the code “GYRA_NC002953” and is thus configured to hybridize to sequence encoding the gyrA gene, and the extraction sequence (SEQ ID NO.: 8) 7005-9668 corresponds to coordinates 7005-9668 of GenBank gi number 49484912, which is a Staphylococcus aureus sequence. One of skill in the art will understand how to determine the exact hybridization coordinates of the primers with respect to the GenBank sequences, given this information. The reference nomenclature in the primer name is selected to provide a reference, and does not necessarily mean that the primer pair has been configured with 100% complementarity to that target site on the reference sequence. One with ordinary skill knows how to obtain individual gene sequences or portions thereof from genomic sequences present in GenBank. In Table 1, Tp=5-propynyluracil; Cp=5-propynylcytosine; *=phosphorothioate linkage; I=inosine. T. GenBank gi numbers for reference sequences of bacteria are shown in Table 2 (below). In some cases, the reference sequences are extractions from bacterial genomic sequences or complements thereof. A description of the primer design is provided herein.

TABLE 1
Primer Pairs for Identification of Staphylococcus

| Primer | | | Forward | | | |
|---|---|---|---|---|---|---|
| Pair | | Forward | SEQ ID | | Reverse | |
| Number | Forward Primer Name | Sequence | NO. | Reverse Primer Name | Sequence | Reverse SEQ ID NO. |
| 258 | RNASEP_SA_31_49_F | GAGGAAAGTCCAT | 255 | RNASEP_SA_358_379_R | ATAAGCCATGTTC | 312 |
| | | GCTCAC | | | TGTTCCATC | |
| 258 | RNASEP_SA_31_49_F | GAGGAAAGTCCAT | 255 | RNASEP_EC_345_362_R | ATAAGCCGGGTTC | 313 |
| | | GCTCAC | | | TGTCG | |
| 258 | RNASEP_SA_31_49_F | GAGGAAAGTCCAT | 255 | RNASEP_BS_363_384_R | GTAAGCCATGTTT | 314 |
| | | GCTCAC | | | TGTTCCATC | |
| 258 | RNASEP_EC_61_77_F | GAGGAAAGTCCGG | 257 | RNASEP_SA_358_379_R | ATAAGCCATGTTC | 312 |
| | | GCTC | | | TGTTCCATC | |
| 258 | RNASEP_EC_61_77_F | GAGGAAAGTCCGG | 257 | RNASEP_EC_345_362_R | ATAAGCCGGGTTC | 313 |
| | | GCTC | | | TGTCG | |
| 258 | RNASEP_EC_61_77_F | GAGGAAAGTCCGG | 257 | RNASEP_BS_363_384_R | GTAAGCCATGTTT | 314 |
| | | GCTC | | | TGTTCCATC | |
| 258 | RNASEP_BS_43_61_F | GAGGAAAGTCCAT | 256 | RNASEP_SA_358_379_R | ATAAGCCATGTTC | 312 |
| | | GCTCGC | | | TGTTCCATC | |
| 258 | RNASEP_BS_43_61_F | GAGGAAAGTCCAT | 256 | RNASEP_EC_345_362_R | ATAAGCCGGGTTC | 313 |
| | | GCTCGC | | | TGTCG | |
| 258 | RNASEP_BS_43_61_F | GAGGAAAGTCCAT | 256 | RNASEP_BS_363_384_R | GTAAGCCATGTTT | 314 |
| | | GCTCGC | | | TGTTCCATC | |
| 259 | RNASEP_BS_43_61_F | GAGGAAAGTCCAT | 256 | RNASEP_BS_363_384_R | GTAAGCCATGTTT | 314 |
| | | GCTCGC | | | TGTTCCATC | |
| 260 | RNASEP_EC_61_77_F | GAGGAAAGTCCGG | 257 | RNASEP_EC_345_362_R | ATAAGCCGGGTTC | 313 |
| | | GCTC | | | TGTCG | |
| 262 | RNASEP_SA_31_49_F | GAGGAAAGTCCAT | 255 | RNASEP_SA_358_379_R | ATAAGCCATGTTC | 312 |
| | | GCTCAC | | | TGTTCCATC | |
| 877 | MECA_Y14051_3774_3802_F | TAAAACAAACTAC | 57 | MECA_Y14051_3828_3854_R | TCCCAATCTAACT | 141 |
| | | GGTAACATTGATC | | | TCCACATACCATC | |
| | | GCA | | | T | |
| 878 | MECA_Y14051_3645_3670_F | TGAAGTAGAAATG | 56 | MECA_Y14051_3690_3719_R | TGATCCTGAATGT | 140 |
| | | ACTGAACGTCCGA | | | TTATATCTTTAAC | |
| | | | | | GCCT | |
| 879 | MECA_Y14051_4507_4530_F | TCAGGTACTGCTA | 58 | MECA_Y14051_4555_4581_R | TGGATAGACGTCA | 142 |
| | | TCCACCCTCAA | | | TATGAAGGTGTGC | |
| | | | | | T | |
| 880 | MECA_Y14051_4510_4530_F | TGTACTGCTATCC | 59 | MECA_Y14051_4586_4610_R | TATTCTTCGTTAC | 143 |
| | | ACCCTCAA | | | TCATGCCATACA | |
| 881 | MECA_Y14051_4669_4698_F | TCACCAGGTTCAA | 61 | MECA_Y14051_4765_4793_R | TAACCACCCCAAG | 146 |
| | | CTCAAAAAATATT | | | ATTTATCTTTTTG | |
| | | AACA | | | CCA | |
| 882 | MECA_Y14051_4520_4530P_F | TCpCpACpCpCpT | 60 | MECA_Y14051_4590_4600P_R | TpACpTpCpATpG | 144 |
| | | pCpAA | | | CpCpA | |
| 883 | MECA_Y14051_4520_4530P_F | TCpCpACpCpCpT | 60 | MECA_Y14051_4600_4610_R | TpATpTpCpTpTp | 145 |
| | | pCpAA | | | CpGTpT | |
| 2056 | MECI- | TTTACACATATCG | 62 | MECI- | TTGTGATATGGAG | 147 |
| | R_NC003923- | TGAGCAATGAACT | | R_NC003923- | GTGTAGAAGGTGT | |
| | 41798- | GA | | 41798- | TA | |
| | 41609_33_60_F | | | 41609_86_113_R | | |
| 2057 | AGR- | TCACCAGTTTGCC | 191 | AGR- | ACCTGCATCCCTA | 266 |
| | III_NC003923- | ACGTATCTTCAA | | III_NC003923- | AACGTACTTGC | |
| | 2108074- | | | 2108074- | | |
| | 2109507_1_23_F | | | 2109507_56_79_R | | |
| 2058 | AGR- | TGAGCTTTTAGTT | 192 | AGR- | TACTTCAGCTTCG | 267 |
| | III_NC003923- | GACTTTTTCAACA | | III_NC003923- | TCCAATAAAAAAT | |
| | 2108074- | GC | | 2108074- | CACAAT | |
| | 2109507_569_596_F | | | 2109507_622_653_R | | |
| 2059 | AGR- | TTTCACACAGCGT | 193 | AGR- | TGTAGGCAAGTGC | 268 |
| | III_NC003923- | GTTTATAGTTCTA | | III_NC003923- | ATAAGAAATTGAT | |
| | 2108074- | CCA | | 2108074- | ACA | |
| | 2109507_1024_1052_F | | | 2109507_1070_1098_R | | |
| 2060 | AGR- | TGGTGACTTCATA | 217 | AGR- | TCCCCATTTAATA | 292 |
| | I_AJ617706_622_651_F | ATGGATGAAGTTG | | I_AJ617706_694_726_R | ATTCCACCTACTA | |
| | | AAGT | | | TCACACT | |
| 2061 | AGR- | TGGGATTTTAAAA | 218 | AGR- | TGGTACTTCAACT | 293 |
| | I_AJ617706_580_611_F | AACATTGGTAACA | | I_AJ617706_626_655_R | TCATCCATTATGA | |
| | | TCGCAG | | | AGTC | |
| 2062 | AGR- | TCTTGCAGCAGTT | 219 | AGR- | TTGTTTATTGTTT | 294 |
| | II_NC002745- | TATTTGATGAACC | | II-NC002745- | CCATATGCTACAC | |
| | 2079448- | TAAAGT | | 2079448- | ACTTTC | |
| | 2080879_620_651_F | | | 2080879_700_731_R | | |
| 2063 | AGR- | TGTACCCGCTGAA | 220 | AGR- | TCGCCATAGCTAA | 1 |
| | II_NC002745- | TTAACGAATTTAT | | II_NC002745- | GTTGTTTATTGTT | |
| | 2079448- | ACGAC | | 2079448- | TCCAT | |
| | 2080879_649_679_F | | | 2080879_715_745_R | | |
| 2064 | AGR- | TGGTATTCTATTT | 221 | AGR- | TGCGCTATCAACG | 296 |
| | IV_AJ617711_931_961_F | TGCTGATAATGAC | | IV_AJ617711_1004_1035_R | ATTTTGACAATAT | |
| | | CTCGC | | | ATGTGA | |
| 2065 | AGR- | TGGCACTCTTGCC | 222 | AGR- | TCCCATACCTATG | 297 |
| | IV_AJ617711_250_283_F | TTTAATATTAGTA | | IV_AJ617711_309_335_R | GCGATAACTGTCA | |
| | | AACTATCA | | | T | |
| 2066 | BLAZ_NC002952 | TCCACTTATCGCA | 223 | BLAZ_NC002952 | TGGCCACTTTTAT | 280 |
| | (1913827.1914672)_68_68_F | AATGGAAAATTAA | | (1913827 . . . 1914672)_68_68_4_R | CAGCAACCTTACA | |
| | | GCAA | | | GTC | |
| 2067 | BLAZ_NC002952 | TGCACTTATCGCA | 224 | BLAZ_NC002952 | TAGTCTTTTGGAA | 281 |
| | (1913827.1914672)_68_68_2_F | AATGGAAAATTAA | | (1913827 . . . 1914672)_68_68_3_R | CACCGTCTTTAAT | |
| | | GCAA | | | TAAAGT | |
| 2068 | BLAZ_NC002952 | TGATACTTCAACG | 225 | BLAZ_NC002952 | TGGAACACCGTCT | 282 |
| | (1913827 . . . 1914672)_68_68_3_F | CCTGCTGCTTTC | | (1913827 . . . 1914672)_68_68_3_R | TTAATTAAAGTAT | |
| | | | | | CTCC | |
| 2069 | BLAZ_NC002952 | TATACTTCAACGC | 226 | BLAZ_NC002952 | TCTTTTCTTTGCT | 283 |
| | (1913827 . . . 1914672_68_68_4_F | CTGCTGCTTTC | | (1913827 . . . 1914672)_68_68_4_R | TAATTTTCCATTT | |
| | | | | | GCGAT | |
| 2070 | BLAZ_NC002952 | TGCAATTGCTTTA | 227 | BLAZ_NC002952 | TTACTTCCTTACC | 284 |
| | (1913827 . . . 1914672)_1_33_F | GTTTTAAGTGCAT | | (1913827 . . . 1914672)_34_67_R | ACTTTTAGTATCT | |
| | | GTAATTC | | | AAAGCATA | |
| 2071 | BLAZ_NC002952 | TCCTTGCTTTAGT | 228 | BLAZ_NC002952 | TGGGGACTTCCTT | 285 |
| | (1913827 . . . 1914672)_3_34_F | TTTAAGTGCATGT | | (1913827 . . . 1914672)_40_68_R | ACCACTTTTAGTA | |
| | | AATTCAA | | | TCTAA | |
| 2072 | BSA- | TAGCGAATGTGGC | 194 | BSA- | TGCAAGGGAAACC | 269 |
| | A_NC003923- | TTTACTTCACAAT | | A_NC003923- | TAGAATTACAAAC | |
| | 1304065- | T | | 1304065- | CCT | |
| | 1303589_99_125_F | | | 1303589_165_193_R | | |
| 2073 | BSA- | ATCAATTTGGTGG | 195 | BSA- | TGCATAGGGAAGG | 270 |
| | A_NC003923- | CCAAGAACCTGG | | A_NC003923- | TAACACCATAGTT | |
| | 1304065- | | | 1304065- | | |
| | 1303589_194_218_F | | | 1303589_253_278_R | | |
| 2074 | BSA- | TTGACTGCGGCAC | 196 | BSA- | TAACAACGTTACC | 271 |
| | A_NC003923- | AACACGGAT | | A_NC003923- | TTCGCGATCCACT | |
| | 1304065- | | | 1304065- | AA | |
| | 1303589_328_349_F | | | 1303589_388_415_R | | |
| 2075 | BSA- | TGCTATGGTGTTA | 197 | BSA- | TGTTGTGCCGCAG | 272 |
| | A_NC003923- | CCTTCCCTATGCA | | A_NC003923- | TCAAATATCTAAA | |
| | 1304065- | | | 1304065- | TA | |
| | 1303589_253_278_F | | | 1303589_317_344_R | | |
| 2076 | BSA- | TAGCAACAAATAT | 198 | BSA- | TGTGAAGAACTTT | 273 |
| | B_NC003923- | ATCTGAAGCAGCG | | B_NC003923- | CAAATCTGTGAAT | |
| | 1917149- | TACT | | 1917149- | CCA | |
| | 1914156_953_982_F | | | 1914156_1011_1039_R | | |
| 2077 | BSA- | TGAAAAGTATGGA | 199 | BSA- | TCTTCTTGAAAAA | 274 |
| | B_NC003923- | TTTGAACAACTCG | | B_NC003923- | TTGTTGTCCCGAA | |
| | 1917149- | TGAATA | | 1917149- | AC | |
| | 1914156_1050_1081_F | | | 1914156_1109_1136_R | | |
| 2078 | BSA- | TCATTATCATGCG | 200 | BSA- | TGGACTAATAACA | 275 |
| | B_NC003923- | CCAATGAGTGCAG | | B_NC003923- | ATGAGCTCATTGT | |
| | 1917149- | A | | 1917149- | ACTGA | |
| | 1914156_1260_1286_F | | | 1914156_1323_1353_R | | |
| 2079 | BSA- | TTTCATCTTATCG | 201 | BSA- | TGAATATGTAATG | 276 |
| | B_NC003923- | AGGACCCGAAATC | | B_NC003923- | CAAACCAGTCTTT | |
| | 1917149- | GA | | 1917149- | GTCAT | |
| | 1914156_2126_2153_F | | | 1914156_2186_2216_R | | |
| 2080 | ERMA_NC002952- | TCGCTATCTTATC | 28 | ERMA_NC002952- | TGAGTCTACACTT | 114 |
| | 55890- | GTTGAGAAGGGAT | | 55890- | GGCTTAGGATGAA | |
| | 56621_366_392_F | T | | 56621_487_513_R | A | |
| 2081 | ERMA_NC002952- | TAGCTATCTTATC | 294 | ERMA_NC002952- | TGAGCATTTTTAT | 295 |
| | 55890- | GTTGAGAAGGGAT | | 55890- | ATCCATCTCCACC | |
| | 56621_366_395_F | TTGC | | 56621_438_465_R | AT | |
| 2082 | ERMA_NC002952- | TGATCGTTGAGAA | 27 | ERMA_NC002952- | TCTTGGCTTAGGA | 113 |
| | 55890- | GGGATTTGCGAAA | | 55890- | TGAAAATATAGTG | |
| | 56621_374_402_F | AGA | | 56621_473_504_R | GTGGTA | |
| 2083 | ERMA_NC002952- | TGCAAAATCTGCA | 29 | ERMA_NC002952- | TCAATACAGAGTC | 115 |
| | 55890- | ACGAGCTTTGG | | 55890- | TACACTTGGCTTA | |
| | 56621_404_427_F | | | 56621_491_520_R | GGAT | |
| 2084 | ERMA_NC002952- | TCATCCTAAGCCA | 30 | ERMA_NC002952- | TGGACGATATTCA | 116 |
| | 55890- | AGTGTAGACTCTG | | 55890- | CGGTTTACCCACT | |
| | 56621_489_516_F | TA | | 56621_586_615_R | TATA | |
| 2085 | ERMA_NC002952- | TATAAGTGGGTAA | 31 | ERMA_NC002952- | TTGACATTTGCAT | 117 |
| | 55890- | ACCGTGAATATCG | | 55890- | GCTTCAAAGCCTG | |
| | 56621_586_614_F | TGT | | 56621_640_665_R | | |
| 2086 | ERMC_NC005908- | TCTGAACATGATA | 35 | ERMC_NC005908- | TCCGTAGTTTTGC | 121 |
| | 2004- | ATATCTTTGAAAT | | 2004- | ATAATTTATGGTC | |
| | 2738_85_116_F | CGGCTC | | 2738_173_206_R | TATTTCAA | |
| 2087 | ERMC_NC005908- | TCATGATAATATC | 33 | ERMC_NC005908- | TTTATGGTCTATT | 119 |
| | 2004- | TTTGAAATCGGCT | | 2004- | TCAATGGCAGTTA | |
| | 2738_90_120_F | CAGGA | | 2738_160_189_R | CGAA | |
| 2088 | ERMC_NC005908- | TCAGGAAAAGGGC | 34 | ERMC_NC005908- | TATGGTCTATTTC | 120 |
| | 2004- | ATTTTACCCTTG | | 2004- | AATGGCAGTTACG | |
| | 2738_115_139_F | | | 2738_161_187_R | A | |
| 2089 | ERMC_NC005908- | TAATCGTGGAATA | 36 | ERMC_NC005908- | TCAACTTCTGCCA | 122 |
| | 2004- | CGGGTTTGCTA | | 2004- | TTAAAAGTAATGC | |
| | 2738_374_397_F | | | 2738_425_452_R | CA | |
| 2090 | ERMC_NC005908- | TCTTTGAAATCGG | 32 | ERMC_NC005908- | TGATGGTCTATTT | 118 |
| | 2004- | CTCAGGAAAAGG | | 2004- | CAATGGCAGTTAC | |
| | 2738_101_125_F | | | 2738_159_188_R | GAAA | |
| 2091 | ERMB_Y13600- | TGTTGGGAGTATT | 229 | ERMB_Y13600- | TCAACAATCAGAT | 286 |
| | 625- | CCTTACCATTTAA | | 625- | AGATGTCAGACGC | |
| | 1362_291_321_F | GCACA | | 1362_352_380_R | ATG | |
| 2092 | ERMB_Y13600- | TGGAAAGCCATGC | 230 | ERMB_Y13600- | TGCAAGAGCAACC | 287 |
| | 625- | GTCTGACATCT | | 625- | CTAGTGTTCG | |
| | 1362_344_367_F | | | 1362_415_437_R | | |
| 2093 | ERMB_Y13600- | TGGATATTCACCG | 231 | ERMB_Y13600- | TAGGATGAAAGCA | 288 |
| | 625- | AACACTAGGGTTG | | 625- | TTCCGCTGGC | |
| | 1362_404_429_F | | | 1362_471_493_R | | |
| 2094 | ERMB_Y13600- | TAAGCTGCCAGCG | 232 | ERMB_Y13600- | TCATCTGTGGTAT | 289 |
| | 625- | GAATGCTTTC | | 625- | GGCGGGTAAGTT | |
| | 1362_465_487_F | | | 1362_521_545_R | | |
| 2095 | PVLUK_NC003923- | TGAGCTGCATCAA | 39 | PVLUK_NC003923- | TGGAAAACTCATG | 125 |
| | 1529595- | CTGTATTGGATAG | | 1529595- | AAATTAAAGTGAA | |
| | 1531285_688_713_F | | | 1531285_775_804_R | AGGA | |
| 2096 | PVLUK_NC003923- | TGGAACAAAATAG | 37 | PVLUK_NC003923- | TCATTAGGTAAAA | 123 |
| | 1529595- | TCTCTCGGATTTT | | 1529595- | TGTCTGGACATGA | |
| | 1531285_1039_1068_F | GACT | | 1531285_1095_1125_R | TCCAA | |
| 2097 | PVLUK_NC003923- | TGAGTAACATCCA | 40 | PVLUK_NC003923- | TCTCATGAAAAAG | 126 |
| | 1529595- | TATTTCTGCCATA | | 1529595- | GCTCAGGAGATAC | |
| | 1531285_908_936_F | CGT | | 1531285_950_978_R | AAG | |
| 2098 | PVLUK_NC003923- | TCGGAATCTGATG | 38 | PVLUK_NC003923- | TCACACCTGTAAG | 124 |
| | 1529595- | TTGCAGTTGTT | | 1529595- | TGAGAAAAAGGTT | |
| | 1531285_610_633_F | | | 1531285_654_682_R | GAT | |
| 2099 | SA442_NC003923- | TGTCGGTACACGA | 205 | SA442_NC003923- | TTTCCGATGCAAC | 13 |
| | 2538576- | TATTCTTCACGA | | 2538576- | GTAATGAGATTTC | |
| | 2538831_11_35_F | | | 2538831_98_124_R | A | |
| 2100 | SA442_NC003923- | TGAAATCTCATTA | 206 | SA442_NC003923- | TCGTATGACCAGC | 14 |
| | 2538576- | CGTTGCATCGGAA | | 2538576- | TTCGGTACTACTA | |
| | 2538831_98_124_F | A | | 2538831_163_188_R | | |
| 2101 | SA442_NC003923- | TCTCATTACGTTG | 207 | SA442_NC003923- | TTTATGACCAGCT | 15 |
| | 2538576- | CATCGGAAACA | | 2538576- | TCGGTACTACTAA | |
| | 2538831_103_126_F | | | 2538831_161_187_R | A | |
| 2102 | SA442_NC003923- | TAGTACCGAAGCT | 208 | SA442_NC003923- | TGATAATGAAGGG | 96 |
| | 2538576- | GGTCATACGA | | 2538576- | AAACCTTTTTCAC | |
| | 2538831_166_188_F | | | 2538831_231_257_R | G | |
| 2103 | SEA_NC003923- | TGCAGGGAACAGC | 209 | SEA_NC003923- | TCGATCGTGACTC | 97 |
| | 2052219- | TTTAGGCA | | 2052219- | TCTTTATTTTCAG | |
| | 2051456_115_135_F | | | 2051456_173_200_R | TT | |
| 2104 | SEA_NC003923- | TAACTCTGATGTT | 210 | SEA_NC003923- | TGTAATTAACCGA | 98 |
| | 2052219- | TTTGATGGGAAGG | | 2052219- | AGGTTCTGTAGAA | |
| | 2051456_572_598_F | T | | 2051456_621_651_R | GTATG | |
| 2105 | SEA_NC003923- | TGTATGGTGGTGT | 211 | SEA_NC003923- | TAACCGTTTCCAA | 317 |
| | 2052219- | AACGTTACATGAT | | 2052219- | AGGTACTGTATTT | |
| | 2051456_382_414_F | AATAATC | | 2051456_464_492_R | TGT | |
| 2106 | SEA_NC003923- | TTGTATGTATGGT | 212 | SEA_NC003923- | TAACCGTTTCCAA | 318 |
| | 2052219- | GGTGTAACGTTAC | | 2052219- | AGGTACTGTATTT | |
| | 2051456_377_406_F | ATGA | | 2051456_459_492_R | TGTTTACC | |
| 2107 | SEB_NC002758- | TTTCACATGTAAT | 247 | SEB_NC002758- | TCATCTGGTTTAG | 304 |
| | 2135540- | TTTGATATTCGCA | | 2135540- | GATCTGGTTGACT | |
| | 2135140_208_237_F | CTGA | | 2135140_273_298_R | | |
| 2108 | SEB_NC002758- | TATTTCACATGTA | 248 | SEB_NC002758- | TGCAACTCATCTG | 305 |
| | 2135540- | ATTTTGATATTCG | | 2135540- | GTTTAGGATCT | |
| | 2135140_206_235_F | CACT | | 2135140_281_304_R | | |
| 2109 | SEB_NC002758- | TAACAACTCGCCT | 249 | SEB_NC002758- | TGTGCAGGCATCA | 306 |
| | 2135540- | TATGAAACGGGAT | | 2135540- | TGTCATACCAA | |
| | 2135140_402_402_F | ATA | | 2135140_402_402_R | | |
| 2110 | SEB_NC002758- | TTGTATGTATGGT | 250 | SEB_NC002758- | TTACCATCTTCAA | 307 |
| | 2135540- | GGTGTAACTGAGC | | 2135540- | ATACCCGAACAGT | |
| | 2135140_402_402_2_F | A | | 2135140_402_402_2_R | AA | |
| 2111 | SEC_NC003923- | TTAACATGAAGGA | 213 | SEC_NC003923- | TGAGTTTGCACTT | 319 |
| | 851678- | AACCATTTGATA | | 851678- | CAAAAGAAATTGT | |
| | 852768_546_575_F | ATGG | | 852768_620_647_R | GT | |
| 2112 | SEC_NC003923- | TGGAATAACAAAA | 214 | SEC_NC003923- | TCAGTTTGCACTT | 320 |
| | 851678- | CATGAAGGAAACC | | 851678- | CAAAAGAAATTGT | |
| | 852768_537_566_F | ACTT | | 852768_619_647_R | GTT | |
| 2113 | SEC_NC003923- | TGAGTTTAACAGT | 215 | SEC_NC003923- | TCGCCTGGTGCAG | 321 |
| | 851678- | TCACCATATGAAA | | 851678- | GCATCATAT | |
| | 852768_720_749_F | CAGG | | 852768_794_815_R | | |
| 2114 | SEC_NC003923- | TGGTATGATATGA | 216 | SEC_NC003923- | TCTTCACACTTTT | 322 |
| | 851678- | TGCCTGCACCA | | 851678- | AGAATCAACCGTT | |
| | 852768_787_810_F | | | 852768_853_886_R | TTATTGTC | |
| 2115 | SED_M28521_657_682_F | TGGTGGTGAAATA | 183 | SED_M28521_741_770_R | TGTACACCATTTA | 258 |
| | | GATAGGACTGCTT | | | TCCACAAATTGAT | |
| | | | | | TGGT | |
| 2116 | SED_M28521_690_711_F | TGGAGGTGTCACT | 184 | SED_M28521_739_770_R | TGGGCACCATTTA | 259 |
| | | CCACACGAA | | | TCCACAAATTGAT | |
| | | | | | TGGTAT | |
| 2117 | SED_M28521_833_854_F | TTGCACAAGCAAG | 185 | SED_M28521_888_911_R | TCGCGCTGTATTT | 260 |
| | | GCGCTATTT | | | TTCCTCCGAGA | |
| 2118 | SED_M28521_962_987_F | TGGATGTTAAGGG | 186 | SED_M28521_1022_1048_R | TGTCAATATGAAG | 261 |
| | | TGATTTTCCCGAA | | | GTGCTCTGTGGAT | |
| | | | | | A | |
| 2119 | SEA- | TTTACACTACTTT | 233 | SEA- | TCATTTATTTCTT | 290 |
| | SEE_NC002952- | TATTCATTGCCCT | | SEE_NC002952- | CGCTTTTCTCGCT | |
| | 2131289- | AACG | | 2131289- | AC | |
| | 2130703_16_45_F | | | 2130703_71_98_R | | |
| 2120 | SEA- | TGATCATCCGTGG | 234 | SEA- | TAAGCACCATATA | 291 |
| | SEE_NC002952- | TATAACGATTTAT | | SEE_NC002952- | AGTCTACTTTTTT | |
| | 2131289- | TAGT | | 2131289- | CCCTT | |
| | 2130703_249_278_F | | | 2130703_314_344_R | | |
| 2121 | SEE_NC002952- | TGACATGATAATA | 235 | SEE_NC002952- | TCTATAGGTACTG | 323 |
| | 2131289- | ACCGATTGACCGA | | 2131289- | TAGTTTGTTTTCC | |
| | 2130703_409_437_F | AGA | | 2130703_465_494_R | GTCT | |
| 2122 | SEE_NC002952- | TGTTCAAGAGCTA | 236 | SEE_NC002952- | TTTGCACCTTACC | 324 |
| | 2131289- | GATCTTCAGGCAA | | 2131289- | GCCAAAGCT | |
| | 2130703_525_550_F | | | 2130703_586_586_R | | |
| 2123 | SEE_NC002952- | TGTTCAAGAGCTA | 237 | SEE_NC002952- | TACCTTACCGCCA | 325 |
| | 2131289- | GATCTTCAGGCA | | 2131289- | AAGTTAATTGGTA | |
| | 2130703_525_549_F | | | 2130703_586_586_2_R | | |
| 2124 | SEE_NC002952- | TCTGGAGGCACAC | 238 | SEE_NC002952- | TCCGTCTATCCAC | 326 |
| | 2131289- | CAAATAAAACA | | 2131289- | AAGTTAATTGGTA | |
| | 2130703_361_384_F | | | 2130703_444_471_R | CT | |
| 2125 | SEG_NC002758- | TGCTCAACCCGAT | 251 | SEG_NC002758- | TAACTCCTCTTCC | 308 |
| | 1955100- | CCTAAATTAGACG | | 1955100- | TTCAACAGGTGGA | |
| | 1954171_225_251_F | A | | 1954171_321_346_R | | |
| 2126 | SEG_NC002758- | TGGACAATAGACA | 252 | SEG_NC002758- | TGCTTTGTAATCT | 309 |
| | 1955100- | ATCACTTGGATTT | | 1955100- | AGTTCCTGAATAG | |
| | 1954171_623_651_F | ACA | | 1954171_671_702_R | TAACCA | |
| 2127 | SEG_NC002758- | TGGAGGTTGTTGT | 253 | SEG_NC002758- | TGTCTATTGTCGA | 310 |
| | 1955100- | ATGTATGGTGGT | | 1955100- | TTGTTACCTGTAC | |
| | 1954171_540_564_F | | | 1954171_607_635_R | AGT | |
| 2128 | SEG_NC002758- | TACAAAGCAAGAC | 254 | SEG_NC002758- | TGATTCAAATGCA | 311 |
| | 1955100- | ACTGGCTCACTA | | 1955100- | GAACCATCAAACT | |
| | 1954171_694_718_F | | | 1954171_735_762_R | CG | |
| 2129 | SEH_NC002953- | TTGCAACTGCTGA | 239 | SEH_NC002953- | TAGTGTTGTACCT | 327 |
| | 60024- | TTTAGCTCAGA | | 60024- | CCATATAGACATT | |
| | 60977_449_472_F | | | 60977_547_576_R | CAGA | |
| 2130 | SEH_NC002953- | TAGAAATCAAGGT | 240 | SEH_NC002953- | TTCTGAGCTAAAT | 328 |
| | 60024- | GATAGTGGCAATG | | 60024- | CAGCAGTTGCA | |
| | 60977_408_434_F | A | | 60977_450_473_R | | |
| 2131 | SEH_NC002953- | TCTGAATGTCTAT | 241 | SEH_NC002953- | TACCATCTACCCA | 298 |
| | 60024- | ATGGAGGTACAAC | | 60024- | AACATTAGCACCA | |
| | 60977_547_576_F | ACTA | | 60977_608_634_R | A | |
| 2132 | SEH_NC002953- | TTCTGAATGTCTA | 242 | SEH_NC002953- | TAGCACCAATCAC | 299 |
| | 60024- | TATGGAGGTACAA | | 60024- | CCTTTCCTGT | |
| | 60977_546_575_F | CACT | | 60977_594_616_R | | |
| 2133 | SEI_NC002758- | TCAACTCGAATTT | 243 | SEI_NC002758- | TCACAAGGACCAT | 300 |
| | 1957830- | TCAACAGGTACCA | | 1957830- | TATAATCAATGCC | |
| | 1956949_324_349_F | | | 1956949_419_446_R | AA | |
| 2134 | SEI_NC002758- | TTCAACAGGTACC | 244 | SEI_NC002758- | TGTACAAGGACCA | 301 |
| | 1957830- | AATGATTTGATCT | | 1957830- | TTATAATCAATGC | |
| | 1956949_336_363_F | CA | | 1956949_420_447_R | CA | |
| 2135 | SEI_NC002758- | TGATCTCAGAATC | 245 | SEI_NC002758- | TCTGGCCCCTCCA | 302 |
| | 1957830- | TAATAATTGGGAC | | 1957830- | TACATGTATTTAG | |
| | 1956949_356_384_F | GAA | | 1956949_449_474_R | | |
| 2136 | SEI_NC002758- | TCTCAAGGTGATA | 246 | SEI_NC002758- | TGGGTAGGTTTTT | 303 |
| | 1957830- | TTGGTGTAGGTAA | | 1957830- | ATCTGTGACGCCT | |
| | 1956949_223_253_F | CTTAA | | 1956949_290_316_R | T | |
| 2137 | SEJ_AF053140_1307_1332_F | TGTGGAGTAACAC | 187 | SEJ_AF053140_1381_1404_R | TCTAGCGGAACAA | 262 |
| | | TGCATGAAAACAA | | | CAGTTCTGATG | |
| 2138 | SEJ_AF053140_1378_1403_F | TAGCATCAGAACT | 188 | SEJ_AF053140_1429_1458_R | TCCTGAAGATCTA | 263 |
| | | GTTGTTCCGCTAG | | | GTTCTTGAATGGT | |
| | | | | | TACT | |
| 2139 | SEJ_AF053140_1431_1459_F | TAACCATTCAAGA | 189 | SEJ_AF053140_1500_1531_R | TAGTCCTTTCTGA | 264 |
| | | ACTAGATCTTCAG | | | ATTTTACCATCAA | |
| | | GCA | | | AGGTAC | |
| 2140 | SEJ_AF053140_1434_1461_F | TCATTCAAGAACT | 190 | SEJ_AF053140_1521_1549_R | TCAGGTATGAAAC | 265 |
| | | AGATCTTCAGGCA | | | ACGATTAGTCCTT | |
| | | AG | | | TCT | |
| 2141 | TSST_NC002758- | TGGTTTAGATAAT | 66 | TSST_NC002758- | TGTAAAAGCAGGG | 151 |
| | 2137564- | TCCTTAGGATCTA | | 2137564- | CTATAATAAGGAC | |
| | 2138293_206_236_F | TGCGT | | 2138293_278_305_R | TC | |
| 2142 | TSST_NC002758- | TGCGTATAAAAAA | 67 | TSST_NC002758- | TGCCCTTTTGTAA | 152 |
| | 2137564- | CACAGATGGCAGC | | 2137564- | AAGCAGGGCTAT | |
| | 2138293_232_258_F | A | | 2138293_289_313_R | | |
| 2143 | TSST_NC002758- | TCCAAATAAGTGG | 68 | TSST_NC002758- | TACTTTAAGGGGC | 153 |
| | 2137564- | CGTTACAAATACT | | 2137564- | TATCTTTACCATG | |
| | 2138293_382_410_F | GAA | | 2138293_448_478_R | AACCT | |
| 2144 | TSST_NC002758- | TCTTTTACAAAAG | 69 | TSST_NC002758- | TAAGTTCCTTCGC | 154 |
| | 2137564- | GGGAAAAAGTTGA | | 2137564- | TAGTATGTTGGCT | |
| | 2138293_297_325_F | CTT | | 2138293_347_373_R | T | |
| 2145 | ARCC_NC003923- | TCGCCGGCAATGC | 75 | ARCC_NC003923- | TGAGTTAAAATGC | 161 |
| | 2725050- | CATTGGATA | | 2725050- | GATTGATTTCAGT | |
| | 2724595_37_58_F | | | 2724595_97_128_R | TTCCAA | |
| 2146 | ARCC_NC003923- | TGAATAGTGATAG | 72 | ARCC_NC003923- | TCTTCTTCTTTCG | 156 |
| | 2725050- | AACTGTAGGCACA | | 2725050- | TATAAAAAGGACC | |
| | 2724595_131_161_F | ATCGT | | 2724595_214_245_R | AATTGG | |
| 2147 | ARCC_NC003923- | TTGGTCCTTTTTA | 74 | ARCC_NC003923- | TGGTGTTCTAGTA | 160 |
| | 2725050- | TACGAAAGAAGAA | | 2725050- | TAGATTGAGGTAG | |
| | 2724595_218_249_F | GTTGAA | | 2724595_322_353_R | TGGTGA | |
| 2148 | AROE_NC003923- | TTGCGAATAGAAC | 80 | AROE_NC003923- | TCGAATTCAGCTA | 167 |
| | 1674726- | GATGGCTCGT | | 1674726- | AATACTTTTCAGC | |
| | 1674277_371_393_F | | | 1674277_435_464_R | ATCT | |
| 2149 | AROE_NC003923- | TGGGGCTTTAAAT | 79 | AROE_NC003923- | TACCTGCATTAAT | 166 |
| | 1674726- | ATTCCAATTGAAG | | 1674726- | CGCTTGTTCATCA | |
| | 1674277_30_62_F | ATTTTCA | | 1674277_155_181_R | A | |
| 2150 | AROE_NC003923- | TGATGGCAAGTGG | 76 | AROE_NC003923- | TAAGCAATACCTT | 162 |
| | 1674726- | ATAGGGTATAATA | | 1674726- | TACTTGCACCACC | |
| | 1674277_204_232_F | CAG | | 1674277_308_335_R | TG | |
| 2151 | GLPF_NC003923- | TGCACCGGCTATT | 202 | GLPF_NC003923- | TGCAACAATTAAT | 277 |
| | 1296927- | AAGAATTACTTTG | | 1296927- | GCTCCGACAATTA | |
| | 1297391_270_301_F | CCAACT | | 1297391_382_414_R | AAGGATT | |
| 2152 | GLPF_NC003923- | TGGATGGGGATTA | 203 | GLPF_NC003923- | TAAAGACACCGCT | 278 |
| | 1296927- | GCGGTTACAATG | | 1296927- | GGGTTTAAATGTG | |
| | 1297391_27_51_F | | | 1297391_81_108_R | CA | |
| 2153 | GLPF_NC003923- | TAGCTGGCGCGAA | 204 | GLPF_NC003923- | TCACCGATAAATA | 279 |
| | 1296927- | ATTAGGTGT | | 1296927- | AAATACCTAAAGT | |
| | 1297391_239_260_F | | | 1297391_323_359_R | TAATGCCATTG | |
| 2154 | GMK_NC003923- | TACTTTTTTAAAA | 81 | GMK_NC003923- | TGATATTGAACTG | 168 |
| | 1190906- | CTAGGGATGCGTT | | 1190906- | GTGTACCATAATA | |
| | 1191334_91_122_F | TGAAGC | | 1191334_166_197_R | GTTGCC | |
| 2155 | GMK_NC003923- | TGAAGTAGAAGGT | 82 | GMK_NC003923- | TCGCTCTCTCAAG | 169 |
| | 1190906- | GCAAAGCAAGTTA | | 1190906- | TGATCTAAACTTG | |
| | 1191334_240_267_F | GA | | 1191334_305_333_R | GAG | |
| 2156 | GMK_NC003923- | TCACCTCCAAGTT | 83 | GMK_NC003923- | TGGGACGTAATCG | 170 |
| | 1190906- | TAGATCACTTGAG | | 1190906- | TATAAATTCATCA | |
| | 1191334_301_329_F | AGA | | 1191334_403_432_R | TTTC | |
| 2157 | PTA_NC003923- | TCTTGTTTATGCT | 87 | PTA_NC003923- | TGGTACACCTGGT | 172 |
| | 628885- | GGTAAAGCAGATG | | 628885- | TTCGTTTTGATGA | |
| | 629355_237_263_F | G | | 629355_314_345_R | TTTGTA | |
| 2158 | PTA_NC003923- | TGAATTAGTTCAA | 84 | PTA_NC003923- | TGCATTGTACCGA | 171 |
| | 628885- | TCATTTGTTGAAC | | 628885- | AGTAGTTCACATT | |
| | 629355_141_171_F | GACGT | | 629355_211_239_R | GTT | |
| 2159 | PTA_NC003923- | TCCAAACCAGGTG | 88 | PTA_NC003923- | TGTTCTGGATTGA | 175 |
| | 628885- | TATCAAGAACATC | | 628885- | TTGCACAATCACC | |
| | 629355_328_356_F | AGG | | 629355_393_422_R | AAAG | |
| 2160 | TPI_NC003923- | TGCAAGTTAAGAA | 89 | TPI_NC003923- | TGAGATGTTGATG | 176 |
| | 830671- | AGCTGTTGCAGGT | | 830671- | ATTTACCAGTTCC | |
| | 831072_131_160_F | TTAT | | 831072_209_239_R | GATTG | |
| 2161 | TPI_NC003923- | TCCCACGAAACAG | 90 | TPI_NC003923- | TGGTACAACATCG | 177 |
| | 830671- | ATGAAGAAATTAA | | 830671- | TTAGCTTTACCAC | |
| | 831072_1_34_F | CAAAAAAG | | 831072_97_129_R | TTTCACG | |
| 2162 | TPI_NC003923- | TCAAACTGGGCAA | 91 | TPI_NC003923- | TGGCAGCAATAGT | 178 |
| | 830671- | TCGGAACTGGTAA | | 830671- | TTGACGTACAAAT | |
| | 831072_199_227_F | ATC | | 831072_253_286_R | GCACACAT | |
| 2163 | YQI_NC003923- | TGAATTGCTGCTA | 93 | YQI_NC003923- | TCGCCAGCTAGCA | 180 |
| | 378916- | TGAAAGGTGGCTT | | 378916- | CGATGTCATTTTC | |
| | 379431_142_167_F | | | 379431_259_284_R | | |
| 2164 | YQI_NC003923- | TACAACATATTAT | 95 | YQI_NC003923- | TTCGTGCTGGATT | 182 |
| | 378916- | TAAAGAGACGGGT | | 378916- | TTGTCCTTGTCCT | |
| | 379431_44_77_F | TTGAATCC | | 379431_120_145_R | | |
| 2165 | YQI_NC003923- | TCCAGCACGAATT | 92 | YQI_NC003923- | TCCAACCCAGAAC | 179 |
| | 378916- | GCTGCTATGAAAG | | 378916- | CACATACTTTATT | |
| | 379431_135_160_F | | | 379431_193_221_R | CAC | |
| 2166 | YQI_NC003923- | TAGCTGGCGGTAT | 94 | YQI_NC003923- | TCCATCTGTTAAA | 181 |
| | 378916- | GGAGAATATGTCT | | 378916- | CCATCATATACCA | |
| | 379431_275_300_F | | | 379431_364_396_R | TGCTATC | |
| 2167 | BLAZ_(1913827 . . . 1914672)_546_575_F | TCCACTTATCGCA | 223 | BLAZ_(1913827 . . . 1914672)_655_683_R | TGGCCACTTTTAT | 280 |
| | | AATGGAAAATTAA | | | CAGCAACCTTACA | |
| | | GCAA | | | GTC | |
| 2168 | BLAZ_(1913827 . . . 1914672)_546_575_2_F | TGCACTTATCGCA | 224 | BLAZ_(1913827 . . . 1914672)_628_659_R | TAGTCTTTTGGAA | 281 |
| | | AATGGAAAATTAA | | | CACCGTCTTTAAT | |
| | | GCAA | | | TAAAGT | |
| 2169 | BLAZ_(1913827 . . . 1914672)_507_531_F | TGATACTTCAACG | 225 | BLAZ_(1913827 . . . 1914672)_622_651_R | TGGAACACCGTCT | 282 |
| | | CCTGCTGCTTTC | | | TTAATTAAAGTAT | |
| | | | | | CTCC | |
| 2170 | BLAZ_(1913827 . . . 1914672)_508_531_F | TATACTTCAACGC | 226 | BLAZ_(1913827 . . . 1914672)_553_583_R | TCTTTTCTTTGCT | 283 |
| | | CTGCTGCTTTC | | | TAATTTTCCATTT | |
| | | | | | GCGAT | |
| 2171 | BLAZ_(1913827 . . . 1914672)_24_56_F | TGCAATTGCTTTA | 227 | BLAZ_(1913827 . . . 1914672)_121_154_R | TTACTTCCTTACC | 284 |
| | | GTTTTAAGTGCAT | | | ACTTTTAGTATCT | |
| | | GTAATTC | | | AAAGCATA | |
| 2172 | BLAZ_(1913827 . . . 1914672)_26_58_F | TCCTTGCTTTAGT | 228 | BLAZ_(1913827 . . . 1914672)_127_157_R | TGGGGACTTCCTT | 285 |
| | | TTTAAGTGCATGT | | | ACCACTTTTAGTA | |
| | | AATTCAA | | | TCTAA | |
| 2173 | BLAZ_NC002952- | TCCACTTATCGCA | 223 | BLAZ_NC002952- | TGGCCACTTTTAT | 280 |
| | 1913827- | AATGGAAAATTAA | | 1913827- | CAGCAACCTTACA | |
| | 1914672_546_575_F | GCAA | | 1914672_655_683_R | GTC | |
| 2174 | BLAZ_NC002952- | TGCACTTATCGCA | 224 | BLAZ_NC002952- | TAGTCTTTTGGAA | 281 |
| | 1913827- | AATGGAAAATTAA | | 1913827- | CACCGTCTTTAAT | |
| | 1914672_546_575_2_F | GCAA | | 1914672_628_659_R | TAAAGT | |
| 2175 | BLAZ_NC002952- | TGATACTTCAACG | 225 | BLAZ_NC002952- | TGGAACACCGTCT | 282 |
| | 1913827- | CCTGCTGCTTTC | | 1913827- | TTAATTAAAGTAT | |
| | 1914672_507_531_F | | | 1914672_622_651_R | CTCC | |
| 2176 | BLAZ_NC002952- | TATACTTCAACGC | 226 | BLAZ_NC002952- | TCTTTTCTTTGCT | 283 |
| | 1913827- | CTGCTGCTTTC | | 1913827- | TAATTTTCCATTT | |
| | 1914672_508_531_F | | | 1914672_553_583_R | GCGAT | |
| 2177 | BLAZ_NC002952- | TGCAATTGCTTTA | 227 | BLAZ_NC002952- | TTACTTCCTTACC | 284 |
| | 1913827- | GTTTTAAGTGCAT | | 1913827- | ACTTTTAGTATCT | |
| | 1914672_24_56_F | GTAATTC | | 1914672_121_154_R | AAAGCATA | |
| 2178 | BLAZ_NC002952- | TCCTTGCTTTAGT | 228 | BLAZ_NC002952- | TGGGGACTTCCTT | 285 |
| | 1913827- | TTTAAGTGCATGT | | 1913827- | ACCACTTTTAGTA | |
| | 1914672_26_58_F | AATTCAA | | 1914672_127_157_R | TCTAA | |
| 2247 | TUFB_NC002758- | TGTTGAACGTGGT | 46 | TUFB_NC002758- | TGTCACCAGCTTC | 132 |
| | 615038- | CAAATCAAAGTTG | | 615038- | AGCGTAGTCTAAT | |
| | 616222_693_721_F | GTG | | 616222_793_820_R | AA | |
| 2248 | TUFB_NC002758- | TCGTGTTGAACGT | 45 | TUFB_NC002758- | TGTCACCAGCTTC | 132 |
| | 615038- | GGTCAAATCAAAG | | 615038- | AGCGTAGTCTAAT | |
| | 616222_690_716_F | T | | 616222_793_820_R | AA | |
| 2249 | TUFB_NC002758- | TGAACGTGGTCAA | 47 | TUFB_NC002758- | TGTCACCAGCTTC | 132 |
| | 615038- | ATCAAAGTTGGTG | | 615038- | AGCGTAGTCTAAT | |
| | 616222_696_725_F | AAGA | | 616222_793_820_R | AA | |
| 2250 | TUFB_NC002758- | TCCCAGGTGACGA | 42 | TUFB_NC002758- | TGGTTTGTCAGAA | 128 |
| | 615038- | TGTACCTGTAATC | | 615038- | TCACGTTCTGGAG | |
| | 616222_488_513_F | | | 616222_601_630_R | TTGG | |
| 2251 | TUFB_NC002758- | TGAAGGTGGACGT | 51 | TUFB_NC002758- | TAGGCATAACCAT | 135 |
| | 615038- | CACACTCCATTCT | | 615038- | TTCAGTACCTTCT | |
| | 616222_945_972_F | TC | | 616222_1030_1060_R | GGTAA | |
| 2252 | TUFB_NC002758- | TCCAATGCCACAA | 41 | TUFB_NC002758- | TTCCATTTCAACT | 127 |
| | 615038- | ACTCGTGAACA | | 615038- | AATTCTAATAATT | |
| | 616222_333_356_F | | | 616222_424_459_R | CTTCATCGTC | |
| 2253 | NUC_NC002758- | TCCTGAAGCAAGT | 52 | NUC_NC002758- | TACGCTAAGCCAC | 136 |
| | 894288- | GCATTTACGA | | 894288- | GTCCATATTTATC | |
| | 894974_402_424_F | | | 894974_483_509_R | A | |
| 2254 | NUC_NC002758- | TCCTTATAGGGAT | 53 | NUC_NC002758- | TGTTTGTGATGCA | 137 |
| | 894288- | GGCTATCAGTAAT | | 894288- | TTTGCTGAGCTA | |
| | 894974_53_81_F | GTT | | 894974_165_189_R | | |
| 2255 | NUC_NC002758- | TCAGCAAATGCAT | 54 | NUC_NC002758- | TAGTTGAAGTTGC | 138 |
| | 894288- | CACAAACAGATAA | | 894288- | ACTATATACTGTT | |
| | 894974_169_194_F | | | 894974_222_250_R | GGA | |
| 2256 | NUC_NC002758- | TACAAAGGTCAAC | 55 | NUC_NC002758- | TAAATGCACTTGC | 139 |
| | 894288- | CAATGACATTCAG | | 894288- | TTCAGGGCCATAT | |
| | 894974_316_345_F | ACTA | | 894974_396_421_R | | |
| 2309 | MUPR_X75439_1658_1689_F | TCCTTTGATATAT | 18 | MUPR_X75439_1744_1773_R | TCCCTTCCTTAAT | 101 |
| | | TATGCGATGGAAG | | | ATGAGAAGGAAAC | |
| | | GTTGGT | | | CACT | |
| 2310 | MUPR_X75439_1330_1353_F | TTCCTCCTTTTGA | 17 | MUPR_X75439_1413_1441_R | TGAGCTGGTGCTA | 100 |
| | | AAGCGACGGTT | | | TATGAACAATACC | |
| | | | | | AGT | |
| 2312 | MUPR_X75439_1314_1338_F | TTTCCTCCTTTTG | 16 | MUPR_X75439_1381_1409_R | TATATGAACAATA | 99 |
| | | AAAGCGACGGTT | | | CCAGTTCCTTCTG | |
| | | | | | AGT | |
| 2313 | MUPR_X75439_2486_2516_F | TAATTGGGCTCTT | 21 | MUPR_X75439_2548_2574_R | TTAATCTGGCTGC | 104 |
| | | TCTCGCTTAAACA | | | GGAAGTGAAATCG | |
| | | CCTTA | | | T | |
| 2314 | MUPR_X75439_2547_2572_F | TACGATTTCACTT | 23 | MUPR_X75439_2605_2630_R | TCGTCCTCTCGAA | 109 |
| | | CCGCAGCCAGATT | | | TCTCCGATATACC | |
| 2315 | MUPR_X75439_2666_2696_F | TGCGTACAATACG | 24 | MUPR_X75439_2711_2740_R | TCAGATATAAATG | 110 |
| | | CTTTATGAAATTT | | | GAACAAATGGAGC | |
| | | TAACA | | | CACT | |
| 2316 | MUPR_X75439_2813_2843_F | TAATCAAGCATTG | 25 | MUPR_X75439_2867_2890_R | TCTGCATTTTTGC | 111 |
| | | GAAGATGAAATGC | | | GAGCCTGTCTA | |
| | | ATACC | | | | |
| 2317 | MUPR_X75439_884_914_F | TGACATGGACTCC | 26 | MUPR_X75439_977_1007_R | TGTACAATAAGGA | 112 |
| | | CCCTATATAACTC | | | GTCACCTTATGTC | |
| | | TTGAG | | | CCTTA | |
| 2504 | ARCC_NC003923- | TAGTpGATpAGAA | 73 | ARCC_NC003923- | TCpTpTpTpCpGT | 159 |
| | 2725050- | CpTpGTAGGCpAC | | 2725050- | ATAAAAAGGACpC | |
| | 2724595_135_161P_F | pAATpCpGT | | 2724595_214_239P_R | pAATpTpGG | |
| 2505 | PTA_NC003923- | TCTTGTPTpTpAT | 86 | PTA_NC003923- | TACpACpCpTGGT | 174 |
| | 628885- | GCpTpGGTAAAGC | | 628885- | pTpTpCpGTpTpT | |
| | 629355_237_263P_F | AGATGG | | 629355_314_342P_R | pTpGATGATpTpT | |
| | | | | | pGTA | |
| 2738 | GYRA_NC002953- | TAAGGTATGACAC | 2 | GYRA_NC002953- | TCTTGAGCCATAC | 5 |
| | 7005- | CGGATAAATCATA | | 7005- | GTACCATTGC | |
| | 9668_166_195_F | TAAA | | 9668_265-287_R | | |
| 2739 | GYRA_NC002953- | TAATGGGTAAATA | 3 | GYRA_NC002953- | TATCCATTGAACC | 6 |
| | 7005- | TCACCCTCATGGT | | 7005- | AAAGTTACCTTGG | |
| | 9668_221_249_F | GAC | | 9668_316_343_R | CC | |
| 2740 | GYRA_NC002953- | TAATGGGTAAATA | 3 | GYRA_NC002953- | TAGCCATACGTAC | 7 |
| | 7005- | TCACCCTCATGGT | | 7005- | CATTGCTTCATAA | |
| | 9668_221_249_F | GAC | | 9668_253_283_R | ATAGA | |
| 2741 | GYRA_NC002953- | TCACCCTCATGGT | 4 | GYRA_NC002953- | TCTTGAGCCATAC | 5 |
| | 7005- | GACTCATCTATTT | | 7005- | GTACCATTGC | |
| | 9668_234_261_F | AT | | 9668_265_287_R | | |
| 3004 | TUFB_NC002758- | TACAGGCCGTGTT | 43 | TUFB_NC002758- | TCAGCGTAGTCTA | 129 |
| | 615038- | GAACGTGG | | 615038- | ATAATTTACGGAA | |
| | 616222_684_704_F | | | 616222_778_809_R | CATTTC | |
| 3005 | TUFB_NC002758- | TGCCGTGTTGAAC | 44 | TUFB_NC002758- | TGCTTCAGCGTAG | 130 |
| | 615038- | GTGGTCAAAT | | 615038- | TCTAATAATTTAC | |
| | 616222_688_710_F | | | 616222_783_813_R | GGAAC | |
| 3006 | TUFB_NC002758- | TGTGGTCAAATCA | 49 | TUFB_NC002758- | TGCGTAGTCTAAT | 134 |
| | 615038- | AAGTTGGTGAAGA | | 615038- | AATTTACGGAACA | |
| | 616222_700_726_F | A | | 616222_778_807_R | | |
| 3007 | TUFB_NC002758- | TGGTCAAATCAAA | 50 | TUFB_NC002758- | TGCGTAGTCTAAT | 134 |
| | 615038- | GTTGGTGAAGAA | | 615038- | AATTTACGGAACA | |
| | 616222_702_726_F | | | 616222_778_807_R | TTTC | |
| 3008 | TUFB_NC002758- | TGAACGTGGTCAA | 48 | TUFB_NC002758- | TCACCAGCTTCAG | 133 |
| | 615038- | ATCAAAGTTGGTG | | 615038- | CGTAGTCTAATAA | |
| | 616222_696_726_F | AAGAA | | 616222_785_818_R | TTTACGGA | |
| 3009 | TUFB_NC002758- | TCGTGTTGAACGT | 45 | TUFB_NC002758- | TCTTCAGCGTAGT | 131 |
| | 615038- | GGTCAAATCAAAG | | 615038- | CTAATAATTTACG | |
| | 616222_690_716_F | T | | 616222_778_812_R | GAACATTTC | |
| 3010 | MECI- | TCACATATCGTGA | 63 | MECI- | TGTGATATGGAGG | 148 |
| | R_NC003923- | GCAATGAACTG | | R_NC003923- | TGTAGAAGGTG | |
| | 41798- | | | 41798- | | |
| | 41609_36_59_F | | | 41609_89_112_R | | |
| 3011 | MECI- | TGGGCGTGAGCAA | 64 | MECI- | TGGGATGGAGGTG | 149 |
| | R_NC003923- | TGAACTGATTATA | | R_NC003923- | TAGAAGGTGTTAT | |
| | 41798- | C | | 41798- | CATC | |
| | 41609_40_66_F | | | 41609_81_110_R | | |
| 3012 | MECI- | TGGACACATATCG | 62 | MECI- | TGGGATGGAGGTG | 149 |
| | R_NC003923- | TGAGCAATGAACT | | R_NC003923- | TAGAAGGTGTTAT | |
| | 41798- | GA | | 41798- | CATC | |
| | 41609_33_60_2_F | | | 41609_81_110_R | | |
| 3013 | MECI | TGGGTTTACACAT | 65 | MECI- | TGGGGATATGGAG | 150 |
| | R_NC003923- | ATCGTGAGCAATG | | R_NC003923- | GTGTAGAAGGTGT | |
| | 41798- | AACTGA | | 41798- | TATCATC | |
| | 41609_29_60_F | | | 41609_81_113_R | | |
| 3014 | MUPR_X75439_2490_2514_F | TGGGCTCTTTCTC | 20 | MUPR_X75439_2548_2570_R | TCTGGCTGCGGAA | 103 |
| | | GCTTAAACACCT | | | GTGAAATCGT | |
| 3015 | MUPR_X75439_2490_2513_F | TGGGCTCTTTCTC | 19 | MUPR_X75439_2547_2568_R | TGGCTGCGGAAGT | 102 |
| | | GCTTAAACACC | | | GAAATCGTA | |
| 3016 | MUPR_X75439_2482_2510_F | TAGATAATTGGGC | 22 | MUPR_X75439_2551_2573_R | TAATCTGGCTGCG | 106 |
| | | TCTTTCTCGCTTA | | | GAAGTGAAAT | |
| | | AAC | | | | |
| 3017 | MUPR_X75439_2490_2514_F | TGGGCTCTTTCTC | 20 | MUPR_X75439_2549_2573_R | TAATCTGGCTGCG | 105 |
| | | GCTTAAACACCT | | | GAAGTGAAATCG | |
| 3018 | MUPR_X75439_2482_2510_F | TAGATAATTGGGC | 22 | MUPR_X75439_2559_2589_R | TGGTATATTCGTT | 108 |
| | | TCTTTCTCGCTTA | | | AATTAATCTGGCT | |
| | | AAC | | | GCGGA | |
| 3019 | MUPR_X75439_2490_2514_F | TGGGCTCTTTCTC | 20 | MUPR_X75439_2554_2581_R | TCGTTAATTAATC | 107 |
| | | GCTTAAACACCT | | | TGGCTGCGGAAGT | |
| | | | | | GA | |
| 3020 | AROE_NC003923- | TGATGGCAAGTGG | 76 | AROE_NC003923- | TAAGCAATACCTT | 163 |
| | 1674726- | ATAGGGTATAATA | | 1674726- | TACTTGCACCACC | |
| | 1674277_204_232_F | CAG | | 1674277_309_335_R | T | |
| 3021 | AROE_NC003923- | TGGCGAGTGGATA | 78 | AROE_NC003923- | TTCATAAGCAATA | 165 |
| | 1674726- | GGGTATAATACAG | | 1674726- | CCTTTACTTGCAC | |
| | 1674277_207_232_F | | | 1674277_311_339_R | CAC | |
| 3022 | AROE_NC003923- | TGGCpAAGTpGGA | 77 | AROE_NC003923- | TAAGCAATACCpT | 164 |
| | 1674726- | TpAGGGTpATpAA | | 1674726- | pTpTpACTpTpGC | |
| | 1674277_207_232P_F | TpACpAG | | 1674277_311_335P_R | pACpCpAC | |
| 3023 | ARCC_NC003923- | TCTGAAATGAATA | 71 | ARCC_NC003923- | TCTTCTTCTTTCG | 156 |
| | 2725050- | GTGATAGAACTGT | | 2725050- | TATAAAAAGGACC | |
| | 2724595_124_155_F | AGGCAC | | 2724595_214_245_R | AATTGG | |
| 3024 | ARCC_NC003923- | TGAATAGTGATAG | 72 | ARCC_NC003923- | TCTTCTTTCGTAT | 157 |
| | 2725050- | AACTGTAGGCACA | | 2725050- | AAAAAGGACCAAT | |
| | 2724595_131_161_F | ATCGT | | 2724595_212_242_R | TGGTT | |
| 3025 | ARCC_NC003923- | TGAATAGTGATAG | 72 | ARCC_NC003923- | TGCGCTAATTCTT | 158 |
| | 2725050- | AACTGTAGGCACA | | 2725050- | CAACTTCTTCTTT | |
| | 2724595_131_161_F | ATCGT | | 2724595_232_260_R | CGT | |
| 3026 | PTA_NC003923- | TACAATGCTTGTT | 85 | PTA_NC003923- | TGTTCTTGATACA | 173 |
| | 628885- | TATGCTGGTAAAG | | 628885- | CCTGGTTTCGTTT | |
| | 629355_231_259_F | CAG | | 629355_322_351_R | TGAT | |
| 3027 | PTA_NC003923- | TACAATGCTTGTT | 85 | PTA_NC003923- | TGGTACACCTGGT | 172 |
| | 628885- | TATGCTGGTAAAG | | 628885- | TTCGTTTTGATGA | |
| | 629355_231_259_F | CAG | | 629355_314_345_R | TTTGTA | |
| 3028 | PTA_NC003923- | TCTTGTTTATGCT | 87 | PTA_NC003923- | TGTTCTTGATACA | 173 |
| | 628885- | GGTAAAGCAGATG | | 628885- | CCTGGTTTCGTTT | |
| | 629355_237_263_F | G | | 629355_322_351_R | TGAT | |
| 3105 | TSST1_NC002758.2_35_57_F | TAAGCCCTTTGTT | 329 | TSST1_NC002758.2_146_173_R | TCAGACCCACTAC | 330 |
| | | GCTTGCGACA | | | TATACCAGTCTAG | |
| | | | | | CA | |
| 3106 | TSST1_NC002758.2- | TCGTCATCAGCTA | 70 | TSST1_NC002758.2- | TCACTTTGATATG | 155 |
| | 2137509- | ACTCAAATACATG | | 2137509- | TGGATCCGTCATT | |
| | 2138213_519_546_F | GA | | 2138213_593- | CA | |
| | | | | 620_R | | |
| 3107 | TSST1_NC002758.2_334_357_F | TGCCAACATACTA | 331 | TSST1_NC002758.2_415_445_R | TCCCATGAACCTT | 332 |
| | | GCGAAGGAACT | | | AACTTTTAAAGGT | |
| | | | | | AGTTC | |

As noted above, primer pair name codes for primer pairs listed in Table 1, cross-referenced to corresponding reference sequence, bioagent, and gene information are shown in Table 2. The primer name code typically represents the gene to which the given primer pair is targeted. The primer names also include specific coordinates with respect to a reference sequence to which the primer hybridizes. As exemplified above, this reference sequence is often defined by an extraction of a section of sequence or defined by a GenBank gi number (indicated by extraction coordinates in the primer pair name), or the corresponding complementary sequence of the extraction, or, in cases when no extraction coordinates are listed, to the entire sequence of the GenBank gi number. Gene abbreviations are shown in bold type in the “Gene Name” column of Table 2.

Methods for PCR primer design are well known. One of skill in the art will understand that primer pairs configured to prime amplification of a double stranded sequence are configured and named using one strand of the double stranded sequence as a reference. The forward primer is the primer of the pair that comprises full or partial sequence identity to the one strand of the sequence being used as a reference. The reverse primer is the primer of the pair that comprises reverse complementarity to the one strand being of the sequence being used as a reference.

In one embodiment, the “plus” or “top” strand (the primary sequence as submitted to GenBank) of the nucleic acid to which the primers hybridize is used as a reference when designing primer pairs. In this case, the forward primer will comprise identity and the reverse primer will comprise reverse complementarity, to the sequence listed in GenBank for the reference sequence. In some embodiments, the primer pair is configured using the “minus” or “bottom” strand (reverse complement of the primary sequence as submitted to and listed in GenBank). In this case, the forward primer comprises sequence identity to the minus strand, and thus comprises reverse complementarity to the top strand, the sequence listed in GenBank. Similarly, in this case, the reverse primer comprises reverse complementarity to the minus strang, and thus comprises identity to the top strand.

Herein, when the primer is configured using the minus strand as a reference, the extraction sequence is preferably listed in a descending fashion in the primer name (as in the case of the coordinates 1674726-1674277 of the forward primer pair name AROE_NC003923-1674726-1674277_{—}30_{—}62_F). In this case, the forward primer comprises reverse complementarity to the sequence listed in GenBank for the reference gi number. Thus, in the case of this exemplary primer, the forward primer is configured to hybridize within nucleotides 1674697 and 1674665 of gi number 21281729, which is 30 (the first number in the hybridization coordinates 30-62) nucleotides in the reverse direction from the first coordinate (1674697) listed in the extraction sequence. The hybridization site and region of the reference sequence to which a primer in Table 1 hybridizes can be determined and verified with bioinformatics alignment tools as described below using the primer sequence and the reference gi number provided in Table 2.

To determine the exact primer hybridization coordinates of a given pair of primers on a given bioagent nucleic acid sequence and to determine the sequences, molecular masses and base compositions of an amplification product to be obtained upon amplification of nucleic acid of a known bioagent with known sequence information in the region of interest with a given pair of primers, one with ordinary skill in bioinformatics is capable of obtaining alignments of the primers of the present invention with the GenBank gi number of the relevant nucleic acid sequence of the known bioagent. For example, the reference sequence GenBank gi numbers (Table 2) provide the identities of the sequences which can be obtained from GenBank. Alignments can be done using a bioinformatics tool such as BLASTn provided to the public by NCBI (Bethesda, Md.). Alternatively, a relevant GenBank sequence may be downloaded and imported into custom programmed or commercially available bioinformatics programs wherein the alignment can be carried out to determine the primer hybridization coordinates and the sequences, molecular masses and base compositions of the amplification product. For example, to obtain the hybridization coordinates of primer pair number 2095 (SEQ ID NO.: 39: SEQ ID NO.:125), First the forward primer (SEQ ID NO: 39) is subjected to a BLASTn search on the publicly available NCBI BLAST website. “RefSeq_Genomic” is chosen as the BLAST database since the gi numbers refer to genomic sequences. The BLAST query is then performed. Among the top results returned is a match to GenBank gi number 21281729 (Accession Number NC_{—}003923). The result shown below, indicates that the forward primer hybridizes to positions 1530282 . . . 1530307 of the genomic sequence of Staphylococcus aureus subsp. aureus MW2 (represented by gi number 21281729).

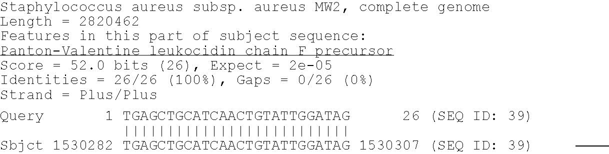

The hybridization coordinates of the reverse primer (SEQ ID NO: 125) can be determined in a similar manner and thus, the bioagent identifying amplicon can be defined in terms of genomic coordinates. The query/subject arrangement of the result would be presented in Strand=Plus/Minus format because the reverse strand hybridizes to the reverse complement of the genomic sequence. The preceding sequence analyses are well known to one with ordinary skill in bioinformatics and thus, Table 2 contains sufficient information to determine the primer hybridization coordinates of any of the primers of Table 1 to the applicable reference sequences described therein.

TABLE 2
Primer Name Codes and Reference Sequences

| | | | Reference |
|---|---|---|---|
| | | | GenBank gi |
| Primer name code | Gene Name | Organism | number |
| RNASEP BDP | RNase P (ribonuclease P) | Bordetella | 33591275 |
| | | pertussis | |
| RNASEP_BKM | RNase P (ribonuclease P) | Burkholderia | 53723370 |
| | | mallei | |
| RNASEP_BS | RNase P (ribonuclease P) | Bacillus subtilis | 16077068 |
| RNASEP CLB | RNase P (ribonuclease P) | Clostridium | 18308982 |
| | | perfringens | |
| RNASEP EC | RNase P (ribonuclease P) | Escherichia coli | 16127994 |
| RNASEP_RKP | RNase P (ribonuclease P) | Rickettsia | 15603881 |
| | | prowazekii | |
| RNASEP SA | RNase P (ribonuclease P) | Staphylococcus | 15922990 |
| | | aureus | |
| RNASEP VBC | RNase P (ribonuclease P) | Vibrio cholerae | 15640032 |
| ICD CXB | icd (isocitrate dehydrogenase) | Coxiella burnetii | 29732244 |
| IS1111A | multi-locus IS1111A insertion element | Acinetobacter | 29732244 |
| | | baumannii | |
| OMPA AY485227 | ompA (outer membrane protein A) | Rickettsia | 40287451 |
| | | prowazekii | |
| OMPB_RKP | ompB (outer membrane protein B) | Rickettsia | 15603881 |
| | | prowazekii | |
| GLTA_RKP | gltA (citrate synthase) | Vibrio cholerae | 15603881 |
| TOXR VBC | toxR (transcription regulator toxR) | Francisella | 15640032 |
| | | tularensis | |
| ASD_FRT | asd (Aspartate semialdehyde | Francisella | 56707187 |
| | dehydrogenase) | tularensis | |
| GALE_FRT | galE (UDP-glucose 4-epimerase) | Shigella flexneri | 56707187 |
| IPAH SGF | ipaH (invasion plasmid antigen) | Campylobacter | 30061571 |
| | | jejuni | |
| HUPB CJ | hupB (DNA-binding protein Hu-beta) | Coxiella burnetii | 15791399 |
| MUPR_X75439 | mupR (mupriocin resistance gene) | Staphylococcus | 438226 |
| | | aureus | |
| PARC X95819 | parC (topoisomerase IV) | Acinetobacter | 1212748 |
| | | baumannii | |
| SED_M28521 | sed (enterotoxin D) | Staphylococcus | 1492109 |
| | | aureus | |
| SEJ AF053140 | sej (enterotoxin J) | Staphylococcus | 3372540 |
| | | aureus | |
| AGR-III NC003923 | agr-III (accessory gene regulator-III) | Staphylococcus | 21281729 |
| | | aureus | |
| ARCC_NC003923 | arcC (carbamate kinase) | Staphylococcus | 21281729 |
| | | aureus | |
| AROE_NC003923 | aroE (shikimate 5-dehydrogenase | Staphylococcus | 21281729 |
| | | aureus | |
| BSA-A NC003923 | bsa-a (glutathione peroxidase) | Staphylococcus | 21281729 |
| | | aureus | |
| BSA-B_NC003923 | bsa-b (epidermin biosynthesis protein | Staphylococcus | 21281729 |
| | EpiB) | aureus | |
| GLPF NC003923 | glpF (glycerol transporter) | Staphylococcus | 21281729 |
| | | aureus | |
| GMK NC003923 | gmk (guanylate kinase) | Staphylococcus | 21281729 |
| | | aureus | |
| MECI-R_NC003923 | mecR1 (truncated methicillin | Staphylococcus | 21281729 |
| | resistance protein) | aureus | |
| PTA NC003923 | pta (phosphate acetyltransferase) | Staphylococcus | 21281729 |
| | | aureus | |
| PVLUK_NC003923 | pvluk (Panton-Valentine leukocidin | Staphylococcus | 21281729 |
| | chain F precursor) | aureus | |
| SA442 NC003923 | sa442 gene | Staphylococcus | 21281729 |
| | | aureus | |
| SEA NC003923 | sea (staphylococcal enterotoxin A | Staphylococcus | 21281729 |
| | precursor) | aureus | |
| SEC_NC003923 | sec4 (enterotoxin type C precursor) | Staphylococcus | 21281729 |
| | | aureus | |
| TPI NC003923 | tpi (triosephosphate isomerase) | Staphylococcus | 21281729 |
| | | aureus | |
| YQI_NC003923 | yqi (acetyl-CoA C-acetyltransferase | Staphylococcus | 21281729 |
| | homologue) | aureus | |
| AGR-II NC002745 | agr-II (accessory gene regulator-II) | Staphylococcus | 29165615 |
| | | aureus | |
| AGR-I AJ617706 | agr-I (accessory gene regulator-I) | Staphylococcus | 46019543 |
| | | aureus | |
| AGR-IV_AJ617711 | agr-IV (accessory gene regulator-III) | Staphylococcus | 46019563 |
| | | aureus | |
| BLAZ NC002952 | blaZ (beta lactamase III) | Staphylococcus | 49482253 |
| | | aureus | |
| ERMA_NC002952 | ermA (rRNA methyltransferase A) | Staphylococcus | 49482253 |
| | | aureus | |
| ERMB Y13600 | ermB (rRNA methyltransferase B) | Staphylococcus | 49482253 |
| | | aureus | |
| SEA-SEE NC002952 | sea (staphylococcal enterotoxin A | Staphylococcus | 49482253 |
| | precursor) | aureus | |
| SEA-SEE NC002952 | sea (staphylococcal enterotoxin A | Staphylococcus | 49482253 |
| | precursor) | aureus | |
| SEE NC002952 | sea (staphylococcal enterotoxin A | Staphylococcus | 49482253 |
| | precursor) | aureus | |
| SEH_NC002953 | seh (staphylococcal enterotoxin H) | Staphylococcus | 49484912 |
| | | aureus | |
| ERMC_NC005908 | ermC (rRNA methyltransferase C) | Staphylococcus | 49489772 |
| | | aureus | |
| NUC NC002758 | nuc (staphylococcal nuclease) | Staphylococcus | 15922990 |
| | | aureus | |
| SEB_NC002758 | seb (enterotoxin type B precursor) | Staphylococcus | 57634611 |
| | | aureus | |
| SEG NC002758 | seg (staphylococcal enterotoxin G) | Staphylococcus | 57634611 |
| | | aureus | |
| SEI_NC002758 | sei (staphylococcal enterotoxin I) | Staphylococcus | 57634611 |
| | | aureus | |
| TSST_NC002758 | tsst (toxic shock syndrome toxin-1) | Staphylococcus | 15922990 |
| | | aureus | |
| TUFB NC002758 | tufB (Elongation factor Tu) | Staphylococcus | 15922990 |
| | | aureus | |
| TSST1_NC002758.2 | tsst (toxic shock syndrome toxin-1) | Staphylococcus | 57634611 |
| | | aureus | |

### Example 2

### Sample Preparation and PCR

Samples were processed to obtain bacterial genomic material using a Qiagen QIAamp Virus BioRobot MDx Kit (Valencia, Calif. 91355). Resulting genomic material was amplified using an MJ Thermocycler Dyad unit (BioRad laboratories, Inc., Hercules, Calif. 94547) and the amplicons were characterized on a Bruker Daltonics MicroTOF instrument (Billerica, Mass. 01821). The resulting molecular mass measurements were converted to base compositions and were queried into a database having base compositions indexed with primer pairs and bioagents.

All PCR reactions were assembled in 50.micro.L reaction volumes in a 96-well microtiter plate format using a Packard MPII liquid handling robotic platform (Perkin Elmer, Bostan, Mass. 02118) and M.J. Dyad thermocyclers (BioRad, Inc., Hercules, Calif. 94547). The PCR reaction mixture consisted of 4 units of Amplitaq Gold, 1× buffer II (Applied Biosystems, Foster City, Calif.), 1.5 mM MgCl.sub.2, 0.4 M betaine, 800.micro.M dNTP mixture and 250 nM of each primer. The following typical PCR conditions were used: 95.deg.C for 10 min followed by 8 cycles of 95.deg.C for 30 seconds, 48.deg.C for 30 seconds, and 72.deg.C 30 seconds with the 48.deg.C annealing temperature increasing 0.9.deg.C with each of the eight cycles. The PCR was then continued for 37 additional cycles of 95.deg.C for 15 seconds, 56.deg.C for 20 seconds, and 72.deg.C 20 seconds. Those ordinarily skilled in the art will understand PCR reactions.

### Example 3

### Solution Capture Purification of PCR Products for Mass Spectrometry with Ion Exchange Resin-Magnetic Beads

For solution capture of nucleic acids with ion exchange resin linked to magnetic beads, 25 micro.l of a 2.5 mg/mL suspension of BioClone amine terminated supraparamagnetic beads (San Diego, Calif. 92126) were added to 25 to 50.micro.l of a PCR (or RT-PCR) reaction containing approximately 10 μM of an amplicon. The above suspension was mixed for approximately 5 minutes by vortexing or pipetting, after which the liquid was removed after using a magnetic separator. The beads containing bound PCR amplicon were then washed three times with 50 mM ammonium bicarbonate/50% MeOH or 100 mM ammonium bicarbonate/50% MeOH, followed by three more washes with 50% MeOH. The bound PCR amplicon was eluted with a solution of 25 mM piperidine, 25 mM imidazole, 35% MeOH which included peptide calibration standards.

### Example 4

### Mass Spectrometry and Base Composition Analysis

The ESI-FTICR mass spectrometer is based on a Bruker Daltonics (Billerica, Mass.) Apex II 70e electrospray ionization Fourier transform ion cyclotron resonance mass spectrometer that employs an actively shielded 7 Tesla superconducting magnet. The active shielding constrains the majority of the fringing magnetic field from the superconducting magnet to a relatively small volume. Thus, components that might be adversely affected by stray magnetic fields, such as CRT monitors, robotic components, and other electronics, can operate in close proximity to the FTICR spectrometer. All aspects of pulse sequence control and data acquisition were performed on a 600 MHz Pentium II data station running Bruker's Xmass software under Windows NT 4.0 operating system. Sample aliquots, typically 15.micro.l, were extracted directly from 96-well microtiter plates using a CTC HTS PAL autosampler (LEAP Technologies, Carrboro, N.C.) triggered by the FTICR data station. Samples were injected directly into a 10.micro.l sample loop integrated with a fluidics handling system that supplies the 100.micro.l/hr flow rate to the ESI source. Ions were formed via electrospray ionization in a modified Analytica (Branford, Conn.) source employing an off axis, grounded electrospray probe positioned approximately 1.5 cm from the metalized terminus of a glass desolvation capillary. The atmospheric pressure end of the glass capillary was biased at 6000 V relative to the ESI needle during data acquisition. A counter-current flow of dry N.sub.2 was employed to assist in the desolvation process. Ions were accumulated in an external ion reservoir comprised of an rf-only hexapole, a skimmer cone, and an auxiliary gate electrode, prior to injection into the trapped ion cell where they were mass analyzed. Ionization duty cycles >99% were achieved by simultaneously accumulating ions in the external ion reservoir during ion detection. Each detection event consisted of 1M data points digitized over 2.3 s. To improve the signal-to-noise ratio (S/N), 32 scans were co-added for a total data acquisition time of 74 s.

The ESI-TOF mass spectrometer is based on a Bruker Daltonics MicroTOF.sup.™. Ions from the ESI source undergo orthogonal ion extraction and are focused in a reflectron prior to detection. The TOF and FTICR are equipped with the same automated sample handling and fluidics described above. Ions are formed in the standard MicroTOF.sup.™ ESI source that is equipped with the same off-axis sprayer and glass capillary as the FTICR ESI source. Consequently, source conditions were the same as those described above. External ion accumulation was also employed to improve ionization duty cycle during data acquisition. Each detection event on the TOF was comprised of 75,000 data points digitized over 75.micro.s.

The sample delivery scheme allows sample aliquots to be rapidly injected into the electrospray source at high flow rate and subsequently be electrosprayed at a much lower flow rate for improved ESI sensitivity. Prior to injecting a sample, a bolus of buffer was injected at a high flow rate to rinse the transfer line and spray needle to avoid sample contamination/carryover. Following the rinse step, the autosampler injected the next sample and the flow rate was switched to low flow. Following a brief equilibration delay, data acquisition commenced. As spectra were co-added, the autosampler continued rinsing the syringe and picking up buffer to rinse the injector and sample transfer line. In general, two syringe rinses and one injector rinse were required to minimize sample carryover. During a routine screening protocol a new sample mixture was injected every 106 seconds. More recently a fast wash station for the syringe needle has been implemented which, when combined with shorter acquisition times, facilitates the acquisition of mass spectra at a rate of just under one spectrum/minute.

Raw mass spectra were post-calibrated with an internal mass standard and deconvoluted to monoisotopic molecular masses. Unambiguous base compositions were derived from the exact mass measurements of the complementary single-stranded oligonucleotides. Quantitative results are obtained by comparing the peak heights with an internal PCR calibration standard present in every PCR well at 500 molecules per well. Calibration methods are commonly owned and disclosed in PCT pre-grant publication number WO 2005/094421, which is incorporated herein by reference in entirety.

### Example 5

### De Novo Determination of Base Composition of Amplicons Using Molecular Mass Modified Deoxynucleotide Triphosphates.

Because the molecular masses of the four natural nucleobases have a relatively narrow molecular mass range (A=313.058, G=329.052, C=289.046, T=304.046, values in Daltons—See Table 3), a persistent source of ambiguity in assignment of base composition can occur as follows: two nucleic acid strands having different base composition may have a difference of about 1 Da when the base composition difference between the two strands is G [symbol] A (−15.994) combined with C [symbol] T (+15.000). For example, one 99-mer nucleic acid strand having a base composition of A.sub.27G.sub.30C.sub.21T.sub.21 has a theoretical molecular mass of 30779.058 while another 99-mer nucleic acid strand having a base composition of A.sub.26G.sub.31C.sub.22T.sub.20 has a theoretical molecular mass of 30780.052 is a molecular mass difference of only 0.994 Da. A 1 Da difference in molecular mass may be within the experimental error of a molecular mass measurement and thus, the relatively narrow molecular mass range of the four natural nucleobases imposes an uncertainty factor in this type of situation. One method for removing this theoretical 1 Da uncertainty factor uses amplification of a nucleic acid with one mass-tagged nucleobase and three natural nucleobases.

Addition of significant mass to one of the 4 nucleobases (dNTPs) in an amplification reaction, or in the primers themselves, will result in a significant difference in mass of the resulting amplicon (greater than 1 Da) arising from ambiguities such as the G [symbol] A combined with C [symbol] T event (Table 3). Thus, the same the G [symbol] A (−15.994) event combined with 5-Iodo-C [symbol] T (−110.900) event would result in a molecular mass difference of 126.894 Da. The molecular mass of the base composition A₂₇G₃₀5-Iodo-C₂₁T₂₁ (33422.958) compared with A.sub.26G.sub.315-Iodo-C.sub.22T.sub.20, (33549.852) provides a theoretical molecular mass difference is +126.894. The experimental error of a molecular mass measurement is not significant with regard to this molecular mass difference. Furthermore, the only base composition consistent with a measured molecular mass of the 99-mer nucleic acid is A.sub.27G.sub.305-Iodo-C.sub.21T.sub.21. In contrast, the analogous amplification without the mass tag has 18 possible base compositions.

TABLE 3
Molecular Masses of Natural Nucleobases and the
Mass-Modified Nucleobase 5-Iodo-C and Molecular
Mass Differences Resulting from Transitions

| Nucleobase | Molecular Mass | Transition | Δ Molecular Mass |
|---|---|---|---|
| A | 313.058 | A-->T | −9.012 |
| A | 313.058 | A-->C | −24.012 |
| A | 313.058 | A-->5-Iodo-C | 101.888 |
| A | 313.058 | A-->G | 15.994 |
| T | 304.046 | T-->A | 9.012 |
| T | 304.046 | T-->C | −15.000 |
| T | 304.046 | T-->5-Iodo-C | 110.900 |
| T | 304.046 | T-->G | 25.006 |
| C | 289.046 | C-->A | 24.012 |
| C | 289.046 | C-->T | 15.000 |
| C | 289.046 | C-->G | 40.006 |
| 5-Iodo-C | 414.946 | 5-Iodo-C-->A | −101.888 |
| 5-Iodo-C | 414.946 | 5-Iodo-C-->T | −110.900 |
| 5-Iodo-C | 414.946 | 5-Iodo-C-->G | −85.894 |
| G | 329.052 | G-->A | −15.994 |
| G | 329.052 | G-->T | −25.006 |
| G | 329.052 | G-->C | −40.006 |
| G | 329.052 | G-->5-Iodo-C | 85.894 |

Mass spectra of bioagent-identifying amplicons can be analyzed using a maximum-likelihood processor, such as is widely used in radar signal processing. This processor first makes maximum likelihood estimates of the input to the mass spectrometer for each primer by running matched filters for each base composition aggregate on the input data. This includes the response to a calibrant for each primer.

The algorithm emphasizes performance predictions culminating in probability-of-detection versus probability-of-false-alarm plots for conditions involving complex backgrounds of naturally occurring organisms and environmental contaminants. Matched filters consist of a priori expectations of signal values given the set of primers used for each of the bioagents. A genomic sequence database is used to define the mass base count matched filters. The database contains the sequences of known bacterial bioagents and includes threat organisms as well as benign background organisms. The latter is used to estimate and subtract the spectral signature produced by the background organisms. A maximum likelihood detection of known background organisms is implemented using matched filters and a running-sum estimate of the noise covariance. Background signal strengths are estimated and used along with the matched filters to form signatures which are then subtracted. The maximum likelihood process is applied to this “cleaned up” data in a similar manner employing matched filters for the organisms and a running-sum estimate of the noise-covariance for the cleaned up data.

The amplitudes of all base compositions of bioagent-identifying amplicons for each primer are calibrated and a final maximum likelihood amplitude estimate per organism is made based upon the multiple single primer estimates. Models of all system noise are factored into this two-stage maximum likelihood calculation. The processor reports the number of molecules of each base composition contained in the spectra. The quantity of amplicon corresponding to the appropriate primer set is reported as well as the quantities of primers remaining upon completion of the amplification reaction.

Base count blurring can be carried out as follows. Electronic PCR can be conducted on nucleotide sequences of the desired bioagents to obtain the different expected base counts that could be obtained for each primer pair. See for example, Schuler, Genome Res. 7:541-50, 1997; or the e-PCR program available from National Center for Biotechnology Information (NCBI, NIH, Bethesda, Md. 20894). One illustrative embodiment uses one or more spreadsheets from a workbook comprising a plurality of spreadsheets (e.g., Microsoft Excel). First in this example, there is a worksheet with a name similar to the workbook name; this worksheet contains the raw electronic PCR data. Second, there is a worksheet named “filtered bioagents base count” that contains bioagent name and base count; there is a separate record for each strain after removing sequences that are not identified with a genus and species and removing all sequences for bioagents with less than 10 strains. Third, there is a worksheet, “Sheetl” that contains the frequency of substitutions, insertions, or deletions for this primer pair. This data is generated by first creating a pivot table from the data in the “filtered bioagents base count” worksheet and then executing an Excel VBA macro. The macro creates a table of differences in base counts for bioagents of the same species, but different strains. One of ordinary skill in the art understands the additional pathways for obtaining similar table differences without undo experimentation.

Application of an exemplary script, involves the user defining a threshold that specifies the fraction of the strains that are represented by the reference set of base counts for each bioagent. The reference set of base counts for each bioagent may contain as many different base counts as are needed to meet or exceed the threshold. The set of reference base counts is defined by taking the most abundant strain's base type composition and adding it to the reference set and then the next most abundant strain's base type composition is added until the threshold is met or exceeded. The current set of data was obtained using a threshold of 55%, which was obtained empirically.

For each base count not included in the reference base count set for that bioagent, the script then proceeds to determine the manner in which the current base count differs from each of the base counts in the reference set. This difference may be represented as a combination of substitutions, Si=Xi, and insertions, Ii=Yi, or deletions, Di=Zi. If there is more than one reference base count, then the reported difference is chosen using rules that aim to minimize the number of changes and, in instances with the same number of changes, minimize the number of insertions or deletions. Therefore, the primary rule is to identify the difference with the minimum sum (Xi+Yi) or (Xi+Zi), e.g., one insertion rather than two substitutions. If there are two or more differences with the minimum sum, then the one that will be reported is the one that contains the most substitutions.

Differences between a base count and a reference composition are categorized as one, two, or more substitutions, one, two, or more insertions, one, two, or more deletions, and combinations of substitutions and insertions or deletions. The different classes of nucleobase changes and their probabilities of occurrence have been delineated in U.S. Patent Application Publication No. 2004209260, which is incorporated herein by reference in entirety.

### Example 6

### Staphylococcus Bacterial Surveillance Panel

The compositions and methods described herein are useful for screening a sample suspected of comprising one or more unknown bioagents to determine the identity of at least one of the bioagents. The compositions and methods provided are also useful for determining population genotype for a sample suspected of comprising a population of bioagents. In one embodiment, the population is a mixed population. The identification of the at least one bioagent or one or more genotypes is accomplished by generating base composition signatures using the methods provided herein for portions of genes shared by two or more members of the Staphylococcus genus. The base composition signatures generated using the methods provided are then compared to a database comprising a plurality of base composition signatures that are indexed to primer pairs used in generating the base composition signatures and bioagents. The plurality of base composition signatures in the database is at least two, is more preferably at least 5, is more preferably still at least 14, is more preferably still at least 19, is more preferably still at least 25 and is more preferably still at least 35. The base composition signatures comprising this plurality identify at least one bioagent when that bioagent's measured and calculated base composition signature is queried against the plurality of base composition signatures comprised in the database.

### Example 7

### Identification of Drug Resistance Genes and Virulence Factors in Staphylococcus aureus

Three primer pair panels, each comprising eight primer pairs, were configured for identification of the Staphylococcus aureus species and for identification of drug resistance genes and virulence factors of Staphylococcus aureus bioagents. These panels are shown in Tables 4-6. The primer sequences in these panels can also be found in Table 1, and are cross-referenced in Tables 4-6 by primer pair numbers, primer pair names, and SEQ ID NOs.

TABLE 4
Panel of Primer Pairs for Identification of Drug Resistance
Genes and Virulence Factors in Staphylococcus aureus

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 879 | MECA_Y14051_4507_4530_F | 58 | MECA_Y14051_4555_4581_R | 142 | mecA |
| 2056 | MECI-R_NC003923-41798- | 62 | MECI-R_NC003923-41798- | 147 | MecI-R |
| | 41609_33_60_F | | 41609_86_113_R | | |
| 2081 | ERMA_NC002952-55890- | 294 | ERMA_NC002952-55890- | 295 | ermA |
| | 56621_366_395_F | | 56621_438_465_R | | |
| 2086 | ERMC_NC005908-2004- | 35 | ERMC_NC005908-2004- | 121 | ermC |
| | 2738_85_116_F | | 2738_173_206_R | | |
| 2095 | PVLUK_NC003923-1529595- | 39 | PVLUK_NC003923-1529595- | 125 | Pv-luk |
| | 1531285_688_713_F | | 1531285_775_804_R | | |
| 2249 | TUFB_NC002758-615038- | 47 | TUFB_NC002758-615038- | 132 | tufB |
| | 616222_696_725_F | | 616222_793_820_R | | |
| 2256 | NUC_NC002758-894288- | 55 | NUC_NC002758-894288- | 139 | Nuc |
| | 894974_316_345_F | | 894974_396_421_R | | |
| 2313 | MUPR_X75439_2486_2516_F | 21 | MUPR_X75439_2548_2574_R | 104 | mupR |

TABLE 5
Panel of Primer Pairs for Identification of Drug Resistance
Genes and Virulence Factors in Staphylococcus aureus

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 879 | MECA_Y14051_4507_4530_F | 58 | MECA_Y14051_4555_4581_R | 142 | mecA |
| 2056 | MECI-R_NC003923-41798- | 62 | MECI-R_NC003923-41798- | 147 | MecI-R |
| | 41609_33_60_F | | 41609_86_113_R | | |
| 2081 | ERMA_NC002952-55890- | 294 | ERMA_NC002952-55890- | 295 | ermA |
| | 56621_366_395_F | | 56621_438_465_R | | |
| 2086 | ERMC_NC005908-2004- | 35 | ERMC_NC005908-2004- | 121 | ermC |
| | 2738_85_116_F | | 2738_173_206_R | | |
| 2095 | PVLUK_NC003923-1529595- | 39 | PVLUK_NC003923-1529595- | 125 | Pv-luk |
| | 1531285_688_713_F | | 1531285_775_804_R | | |
| 2249 | TUFB_NC002758-615038- | 47 | TUFB_NC002758-615038- | 132 | tufB |
| | 616222_696_725_F | | 616222_793_820_R | | |
| 2256 | NUC_NC002758-894288- | 55 | NUC_NC002758-894288- | 139 | Nuc |
| | 894974_316_345_F | | 894974_396_421_R | | |
| 3016 | MUPR_X75439_2482_2510_F | 22 | MUPR_X75439_2551_2573_R | 106 | mupR |

TABLE 6
Panel of Primer Pairs for Identification of Drug Resistance
Genes and Virulence Factors in Staphylococcus aureus

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 879 | MECA_Y14051_4507_4530_F | 58 | MECA_Y14051_4555_4581_R | 142 | mecA |
| 2056 | MECI-R_NC003923-41798- | 62 | MECI-R_NC003923-41798- | 147 | MecI-R |
| | 41609_33_60_F | | 41609_86_113_R | | |
| 2081 | ERMA_NC002952-55890- | 294 | ERMA_NC002952-55890- | 295 | ermA |
| | 56621_366_395_F | | 56621_438_465_R | | |
| 2086 | ERMC_NC005908-2004- | 35 | ERMC_NC005908-2004- | 121 | esrmC |
| | 2738_85_116_F | | 2738_173_206_R | | |
| 2095 | PVLUK_NC003923-1529595- | 39 | PVLUK_NC003923-1529595- | 125 | Pv-luk |
| | 1531285_688_713_F | | 1531285_775_804_R | | |
| 2249 | TUFB_NC002758-615038- | 47 | TUFB_NC002758-615038- | 132 | tufB |
| | 616222_696_725_F | | 616222_793_820_R | | |
| 2256 | NUC_NC002758-894288- | 55 | NUC_NC002758-894288- | 139 | Nuc |
| | 894974_316_345_F | | 894974_396_421_R | | |
| 3106 | TSST1_NC002758.2- | 70 | TSST1_NC002758.2- | 155 | tsst1 |
| | 2137509- | | 2137509-2138213_593- | | |
| | 2138213_519_546_F | | 620_R | | |

Primer pair numbers 2256 and 2249 are confirmation primers configured with the aim of high-level identification of Staphylococcus aureus. The nuc gene is a Staphylococcus aureus-specific marker gene. The tufB gene is a universal housekeeping gene but the bioagent identifying amplicon defined by primer pair number 2249 provides a unique base composition (A43 G28 C19 T35) which distinguishes Staphylococcus aureus from other members of the genus Staphylococcus.

High level methicillin resistance in a given strain of Staphylococcus aureus is indicated by bioagent identifying amplicons defined by primer pair numbers 879 and 2056. Analyses have indicated that primer pair number 879 is not expected to prime S. sciuri homolog or Enterococcus faecalis/faciem ampicillin-resistant PBP5 homologs.

Macrolide and erythromycin resistance in a given strain of Staphylococcus aureus is indicated by bioagent identifying amplicons defined by primer pair numbers 2081 and 2086.

Resistance to mupriocin in a given strain of Staphylococcus aureus is indicated by bioagent identifying amplicons defined by primer pair numbers 2313 and 3016.

In the above panels, virulence in a given strain of Staphylococcus aureus can be indicated by bioagent identifying amplicons defined by primer pair numbers 2095 and 3106. Primer pair number 2095 can identify both the pvl (lukS-PV) gene and the lukD gene which encodes a homologous enterotoxin. A bioagent identifying amplicon of the lukD gene defined by primer pair number 2095 has a six nucleobase length difference relative to the lukS-PV gene. Further, primer pair number 3106 is configured to generate amplicons within the tsst-1 gene, which encodes for shock syndrome toxin, which causes toxic shock syndrome (TSS).

A total of 32 blinded samples of different strains of Staphylococcus aureus were provided by the Center for Disease Control (CDC). Each sample was analyzed by PCR amplification with the first of these eight primer pair panels (shown in Table 4), followed by purification and measurement of molecular masses of the amplification products by mass spectrometry. Base compositions for the amplification products were calculated. The base compositions provide the information summarized above for each primer pair. The results are shown in Tables 7A and 7B.

TABLE 7A
Drug Resistance and Virulence Identified in Blinded
Samples of Various Strains of Staphylococcus aureus
with Primer Pair Nos. 2081, 2086, 2095 and 2256

| | Primer | Primer | | Primer |
|---|---|---|---|---|
| Sample | Pair No. | Pair No. | Primer Pair No. | Pair No. |
| Index No. | 2081 (ermA) | 2086 (ermC) | 2095 (pv-luk) | 2256 (nuc) |
| CDC0010 | − | − | PVL−/lukD+ | + |
| CDC0015 | − | − | PVL+/lukD+ | + |
| CDC0019 | − | + | PVL−/lukD+ | + |
| CDC0026 | + | − | PVL−/lukD+ | + |
| CDC0030 | + | − | PVL−/lukD+ | + |
| CDC004 | − | − | PVL+/lukD+ | + |
| CDC0014 | − | + | PVL+/lukD+ | + |
| CDC008 | − | − | PVL−/lukD+ | + |
| CDC001 | + | − | PVL−/lukD+ | + |
| CDC0022 | + | − | PVL−/lukD+ | + |
| CDC006 | + | − | PVL−/lukD+ | + |
| CDC007 | − | − | PVL−/lukD+ | + |
| CDCVRSA1 | + | − | PVL−/lukD+ | + |
| CDCVRSA2 | + | + | PVL−/lukD+ | + |
| CDC0011 | + | − | PVL−/lukD+ | + |
| CDC0012 | − | − | PVL+/lukD− | + |
| CDC0021 | + | − | PVL−/lukD+ | + |
| CDC0023 | + | − | PVL−/lukD+ | + |
| CDC0025 | + | − | PVL−/lukD+ | + |
| CDC005 | − | − | PVL−/lukD+ | + |
| CDC0018 | + | − | PVL+/lukD− | + |
| CDC002 | − | − | PVL−/lukD+ | + |
| CDC0028 | + | − | PVL−/lukD+ | + |
| CDC003 | − | − | PVL−/lukD+ | + |
| CDC0013 | − | − | PVL+/lukD+ | + |
| CDC0016 | − | − | PVL−/lukD+ | + |
| CDC0027 | + | − | PVL−/lukD+ | + |
| CDC0029 | − | − | PVL+/lukD+ | + |
| CDC0020 | − | + | PVL−/lukD+ | + |
| CDC0024 | − | − | PVL−/lukD+ | + |
| CDC0031 | − | − | PVL−/lukD+ | + |

TABLE 7B
Drug Resistance and Virulence Identified in Blinded Samples of Various Strains
of Staphylococcus aureus with Primer Pair Nos. 2249, 879, 2056, and 2313

| Sample | Primer Pair No. 2249 | Primer Pair No. | Primer Pair No. | Primer Pair No. |
|---|---|---|---|---|
| Index No. | (tufB) | 879 (mecA) | 2056 (mecI-R) | 2313 (mupR) |
| CDC0010 | Staphylococcus aureus | + | + | − |
| CDC0015 | Staphylococcus aureus | − | − | − |
| CDC0019 | Staphylococcus aureus | + | + | − |
| CDC0026 | Staphylococcus aureus | + | + | − |
| CDC0030 | Staphylococcus aureus | + | + | − |
| CDC004 | Staphylococcus aureus | + | + | − |
| CDC0014 | Staphylococcus aureus | + | + | − |
| CDC008 | Staphylococcus aureus | + | + | − |
| CDC001 | Staphylococcus aureus | + | + | − |
| CDC0022 | Staphylococcus aureus | + | + | − |
| CDC006 | Staphylococcus aureus | + | + | + |
| CDC007 | Staphylococcus aureus | + | + | − |
| CDCVRSA1 | Staphylococcus aureus | + | + | − |
| CDCVRSA2 | Staphylococcus aureus | + | + | − |
| CDC0011 | Staphylococcus aureus | − | − | − |
| CDC0012 | Staphylococcus aureus | + | + | − |
| CDC0021 | Staphylococcus aureus | + | + | − |
| CDC0023 | Staphylococcus aureus | + | + | − |
| CDC0025 | Staphylococcus aureus | + | + | − |
| CDC005 | Staphylococcus aureus | + | + | − |
| CDC0018 | Staphylococcus aureus | + | + | − |
| CDC002 | Staphylococcus aureus | + | + | − |
| CDC0028 | Staphylococcus aureus | + | + | − |
| CDC003 | Staphylococcus aureus | + | + | − |
| CDC0013 | Staphylococcus aureus | + | + | − |
| CDC0016 | Staphylococcus aureus | + | + | − |
| CDC0027 | Staphylococcus aureus | + | + | − |
| CDC0029 | Staphylococcus aureus | + | + | − |
| CDC0020 | Staphylococcus aureus | − | − | − |
| CDC0024 | Staphylococcus aureus | + | + | − |
| CDC0031 | Staphylococcus scleiferi | − | − | − |

Upon un-blinding of the samples illustrated in Tables 7A and 7B is was noted that each of the PVL+identifications agreed with PVL+identified in the same samples by standard PCR assays. These results indicate that the panel of eight primer pairs is useful for identification of drug resistance and virulence sub-species characteristics for Staphylococcus aureus. Thus, it is expected that a kit comprising one or more of the members of the panels provided in Tables 4-6, and/or one or more other drug-resistance or virulence-identifying primer pairs provided here will be a useful embodiment.

### Example 8

### Selection and Use of Triangulation Genotyping Analysis Primer Pairs for Staphylococcus aureus

To combine the power of high-throughput mass spectrometric analysis of bioagent identifying amplicons with the sub-species characteristic resolving power provided by triangulation genotyping analysis, two panels of eight triangulation genotyping analysis primer pairs were selected. Each of the primer pairs in these panels is configured to produce bioagent identifying amplicons within one of six different housekeeping genes, which are listed in Tables 8 and 9. The primer sequences are found in Table 1 and are cross-referenced by the primer pair numbers, primer pair names and SEQ ID NOs listed in Tables 8 and 9.

TABLE 8
Primer Pairs for Triangulation Genotyping Analysis of Staphylococcus aureus

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 2146 | ARCC_NC003923-2725050- | 72 | ARCC_NC003923-2725050- | 156 | arcC |
| | 2724595_131_161_F | | 2724595_214_245_R | | |
| 2149 | AROE_NC003923-1674726- | 79 | AROE_NC003923-1674726- | 166 | aroE |
| | 1674277_30_62_F | | 1674277_155_181_R | | |
| 2150 | AROE_NC003923-1674726- | 76 | AROE_NC003923-1674726- | 162 | aroE |
| | 1674277_204_232_F | | 1674277_308_335_R | | |
| 2156 | GMK_NC003923-1190906- | 83 | GMK_NC003923-1190906- | 170 | gmk |
| | 1191334_301_329_F | | 1191334_403_432_R | | |
| 2157 | PTA_NC003923-628885- | 87 | PTA_NC003923-628885- | 172 | pta |
| | 629355_237_263_F | | 629355_314_345_R | | |
| 2161 | TPI_NC003923-830671- | 90 | TPI_NC003923-830671- | 177 | tpi |
| | 831072_1_34_F | | 831072_97_129_R | | |
| 2163 | YQI_NC003923-378916- | 93 | YQI_NC003923-378916- | 180 | yqi |
| | 379431_142_167_F | | 379431_259_284_R | | |
| 2166 | YQI_NC003923-378916- | 94 | YQI_NC003923-378916- | 181 | yqi |
| | 379431_275_300_F | | 379431_364_396_R | | |

TABLE 9
Primer Pairs for Triangulation Genotyping Analysis of Staphylococcus aureus

| | | Forward | | Reverse | |
|---|---|---|---|---|---|
| Primer | | Primer | | Primer | |
| Pair | | (SEQ ID | | (SEQ ID | Target |
| No. | Forward Primer Name | NO:) | Reverse Primer Name | NO:) | Gene |
| 3025 | ARCC_NC003923-2725050- | 72 | ARCC_NC003923-2725050- | 158 | arcC |
| | 2724595_131_161_F | | 2724595_232_260_R | | |
| 2149 | AROE_NC003923-1674726- | 79 | AROE_NC003923-1674726- | 166 | aroE |
| | 1674277_30_62_F | | 1674277_155_181_R | | |
| 2150 | AROE_NC003923-1674726- | 76 | AROE_NC003923-1674726- | 162 | aroE |
| | 1674277_204_232_F | | 1674277_308_335_R | | |
| 2156 | GMK_NC003923-1190906- | 83 | GMK_NC003923-1190906- | 170 | gmk |
| | 1191334_301_329_F | | 1191334_403_432_R | | |
| 2157 | PTA_NC003923-628885- | 87 | PTA_NC003923-628885- | 172 | pta |
| | 629355_237_263_F | | 629355_314_345_R | | |
| 2161 | TPI_NC003923-830671- | 90 | TPI_NC003923-830671- | 177 | tpi |
| | 831072_1_34_F | | 831072_97_129_R | | |
| 2163 | YQI_NC003923-378916- | 93 | YQI_NC003923-378916- | 180 | yqi |
| | 379431_142_167_F | | 379431_259_284_R | | |
| 2166 | YQI_NC003923-378916- | 94 | YQI_NC003923-378916- | 181 | yqi |
| | 379431_275_300_F | | 379431_364_396_R | | |

The samples that were analyzed for drug resistance and virulence in Example 7 were subjected to triangulation genotyping analysis with the first panel of primers listed above. The primer pairs of Table 8 were used to produce amplification products by PCR, which were subsequently purified and measured by mass spectrometry. Base compositions were calculated from the molecular masses and are shown in Tables 10A and 10B.

TABLE 10A
Triangulation Genotyping Analysis of Blinded Samples of Various Strains of
Staphylococcus aureus with Primer Pair Nos. 2146, 2149, 2150 and 2156

| Sample | | Primer Pair No. | Primer Pair No. | Primer Pair No. | Primer Pair No. |
|---|---|---|---|---|---|
| Index No. | Strain | 2146 (arcC) | 2149 (aroE) | 2150 (aroE) | 2156 (gmk) |
| CDC0010 | COL | A44 G24 C18 T29 | A59 G24 C18 T51 | A40 G36 C13 T43 | A50 G30 C20 T32 |
| CDC0015 | COL | A44 G24 C18 T29 | A59 G24 C18 T51 | A40 G36 C13 T43 | A50 G30 C20 T32 |
| CDC0019 | COL | A44 G24 C18 T29 | A59 G24 C18 T51 | A40 G36 C13 T43 | A50 G30 C20 T32 |
| CDC0026 | COL | A44 G24 C18 T29 | A59 G24 C18 T51 | A40 G36 C13 T43 | A50 G30 C20 T32 |
| CDC0030 | COL | A44 G24 C18 T29 | A59 G24 C18 T51 | A40 G36 C13 T43 | A50 G30 C20 T32 |
| CDC004 | COL | A44 G24 C18 T29 | A59 G24 C18 T51 | A40 G36 C13 T43 | A50 G30 C20 T32 |
| CDC0014 | COL | A44 G24 C18 T29 | A59 G24 C18 T51 | A40 G36 C13 T43 | A50 G30 C20 T32 |
| CDC008 | ???? | A44 G24 C18 T29 | A59 G24 C18 T51 | A40 G36 C13 T43 | A50 G30 C20 T32 |
| CDC001 | Mu50 | A45 G23 C20 T27 | A58 G24 C18 T52 | A40 G36 C13 T43 | A51 G29 C21 T31 |
| CDC0022 | Mu50 | A45 G23 C20 T27 | A58 G24 C18 T52 | A40 G36 C13 T43 | A51 G29 C21 T31 |
| CDC006 | Mu50 | A45 G23 C20 T27 | A58 G24 C18 T52 | A40 G36 C13 T43 | A51 G29 C21 T31 |
| CDC0011 | MRSA252 | A45 G24 C18 T28 | A58 G24 C19 T51 | A41 G36 C12 T43 | A51 G29 C21 T31 |
| CDC0012 | MRSA252 | A45 G24 C18 T28 | A58 G24 C19 T51 | A41 G36 C12 T43 | A51 G29 C21 T31 |
| CDC0021 | MRSA252 | A45 G24 C18 T28 | A58 G24 C19 T51 | A41 G36 C12 T43 | A51 G29 C21 T31 |
| CDC0023 | ST:110 | A45 G24 C18 T28 | A59 G24 C18 T51 | A40 G36 C13 T43 | A50 G30 C20 T32 |
| CDC0025 | ST:110 | A45 G24 C18 T28 | A59 G24 C18 T51 | A40 G36 C13 T43 | A50 G30 C20 T32 |
| CDC005 | ST:338 | A44 G24 C18 T29 | A59 G23 C19 T51 | A40 G36 C14 T42 | A51 G29 C21 T31 |
| CDC0018 | ST:338 | A44 G24 C18 T29 | A59 G23 C19 T51 | A40 G36 C14 T42 | A51 G29 C21 T31 |
| CDC002 | ST:108 | A46 G23 C20 T26 | A58 G24 C19 T51 | A42 G36 C12 T42 | A51 G29 C20 T32 |
| CDC0028 | ST:108 | A46 G23 C20 T26 | A58 G24 C19 T51 | A42 G36 C12 T42 | A51 G29 C20 T32 |
| CDC003 | ST:107 | A45 G23 C20 T27 | A58 G24 C18 T52 | A40 G36 C13 T43 | A51 G29 C21 T31 |
| CDC0013 | ST:12 | ND | A59 G24 C18 T51 | A40 G36 C13 T43 | A51 G29 C21 T31 |
| CDC0016 | ST:120 | A45 G23 C18 T29 | A58 G24 C19 T51 | A40 G37 C13 T42 | A51 G29 C21 T31 |
| CDC0027 | ST:105 | A45 G23 C20 T27 | A58 G24 C18 T52 | A40 G36 C13 T43 | A51 G29 C21 T31 |
| CDC0029 | MSSA476 | A45 G23 C20 T27 | A58 G24 C19 T51 | A40 G36 C13 T43 | A50 G30 C20 T32 |
| CDC0020 | ST:15 | A44 G23 C21 T27 | A59 G23 C18 T52 | A40 G36 C13 T43 | A50 G30 C20 T32 |
| CDC0024 | ST:137 | A45 G23 C20 T27 | A57 G25 C19 T51 | A40 G36 C13 T43 | A51 G29 C22 T30 |
| CDC0031 | *** | No product | No product | No product | No product |

TABLE 10B
Triangulation Genotyping Analysis of Blinded Samples of Various Strains of
Staphylococcus aureus with Primer Pair Nos. 2146, 2149, 2150 and 2156

| Sample | | Primer Pair No. | Primer Pair No. | Primer Pair No. | Primer Pair No. |
|---|---|---|---|---|---|
| Index No. | Strain | 2157 (pta) | 2161 (tpi) | 2163 (yqi) | 2166 (yqi) |
| CDC0010 | COL | A32 G25 C23 T29 | A51 G28 C22 T28 | A41 G37 C22 T43 | A37 G30 C18 T37 |
| CDC0015 | COL | A32 G25 C23 T29 | A51 G28 C22 T28 | A41 G37 C22 T43 | A37 G30 C18 T37 |
| CDC0019 | COL | A32 G25 C23 T29 | A51 G28 C22 T28 | A41 G37 C22 T43 | A37 G30 C18 T37 |
| CDC0026 | COL | A32 G25 C23 T29 | A51 G28 C22 T28 | A41 G37 C22 T43 | A37 G30 C18 T37 |
| CDC0030 | COL | A32 G25 C23 T29 | A51 G28 C22 T28 | A41 G37 C22 T43 | A37 G30 C18 T37 |
| CDC004 | COL | A32 G25 C23 T29 | A51 G28 C22 T28 | A41 G37 C22 T43 | A37 G30 C18 T37 |
| CDC0014 | COL | A32 G25 C23 T29 | A51 G28 C22 T28 | A41 G37 C22 T43 | A37 G30 C18 T37 |
| CDC008 | unknown | A32 G25 C23 T29 | A51 G28 C22 T28 | A41 G37 C22 T43 | A37 G30 C18 T37 |
| CDC001 | Mu50 | A33 G25 C22 T29 | A50 G28 C22 T29 | A42 G36 C22 T43 | A36 G31 C19 T36 |
| CDC0022 | Mu50 | A33 G25 C22 T29 | A50 G28 C22 T29 | A42 G36 C22 T43 | A36 G31 C19 T36 |
| CDC006 | Mu50 | A33 G25 C22 T29 | A50 G28 C22 T29 | A42 G36 C22 T43 | A36 G31 C19 T36 |
| CDC0011 | MRSA252 | A32 G25 C23 T29 | A50 G28 C22 T29 | A42 G36 C22 T43 | A37 G30 C18 T37 |
| CDC0012 | MRSA252 | A32 G25 C23 T29 | A50 G28 C22 T29 | A42 G36 C22 T43 | A37 G30 C18 T37 |
| CDC0021 | MRSA252 | A32 G25 C23 T29 | A50 G28 C22 T29 | A42 G36 C22 T43 | A37 G30 C18 T37 |
| CDC0023 | ST:110 | A32 G25 C23 T29 | A51 G28 C22 T28 | A41 G37 C22 T43 | A37 G30 C18 T37 |
| CDC0025 | ST:110 | A32 G25 C23 T29 | A51 G28 C22 T28 | A41 G37 C22 T43 | A37 G30 C18 T37 |
| CDC005 | ST:338 | A32 G25 C24 T28 | A51 G27 C21 T30 | A42 G36 C22 T43 | A37 G30 C18 T37 |
| CDC0018 | ST:338 | A32 G25 C24 T28 | A51 G27 C21 T30 | A42 G36 C22 T43 | A37 G30 C18 T37 |
| CDC002 | ST:108 | A33 G25 C23 T28 | A50 G28 C22 T29 | A42 G36 C22 T43 | A37 G30 C18 T37 |
| CDC0028 | ST:108 | A33 G25 C23 T28 | A50 G28 C22 T29 | A42 G36 C22 T43 | A37 G30 C18 T37 |
| CDC003 | ST:107 | A32 G25 C23 T29 | A51 G28 C22 T28 | A41 G37 C22 T43 | A37 G30 C18 T37 |
| CDC0013 | ST:12 | A32 G25 C23 T29 | A51 G28 C22 T28 | A42 G36 C22 T43 | A37 G30 C18 T37 |
| CDC0016 | ST:120 | A32 G25 C24 T28 | A50 G28 C21 T30 | A42 G36 C22 T43 | A37 G30 C18 T37 |
| CDC0027 | ST:105 | A33 G25 C22 T29 | A50 G28 C22 T29 | A43 G36 C21 T43 | A36 G31 C19 T36 |
| CDC0029 | MSSA476 | A33 G25 C22 T29 | A50 G28 C22 T29 | A42 G36 C22 T43 | A36 G31 C19 T36 |
| CDC0020 | ST:15 | A33 G25 C22 T29 | A50 G28 C21 T30 | A42 G36 C22 T43 | A36 G31 C18 T37 |
| CDC0024 | ST:137 | A33 G25 C22 T29 | A51 G28 C22 T28 | A42 G36 C22 T43 | A37 G30 C18 T37 |
| CDC0031 | *** | A34 G25 C25 T25 | A51 G27 C24 T27 | No product | No product |

Note:
*** The sample CDC0031 was identified as Staphylococcus scleiferi as indicated in Example 7. Thus, the triangulation genotyping primers configured for Staphylococcus aureus would generally not be expected to prime and produce amplification products of this organism. Tables 10A and 10B indicate that amplification products are obtained for this organism only with primer pair numbers 2157 and 2161.

A total of thirteen different genotypes of Staphylococcus aureus were identified according to the unique combinations of base compositions across the eight different bioagent identifying amplicons obtained with the eight primer pairs. These results indicate that the eight primer pair panel is useful for analysis of unknown or newly emerging strains of Staphylococcus aureus, and thus it is expected that a kit comprising one or more of the members of the panels provided in Tables 8 and 9, and/or one or more other Staphylococcus aureus genotyping primer pairs provided herein, will be a useful embodiment.

### Example 9

### Survey of 326 Staphylococcus aureus Clinical Isolates Using Primers To Drug Resistance/Virulance and Triangulation Genotyping Analysis Primer Pairs

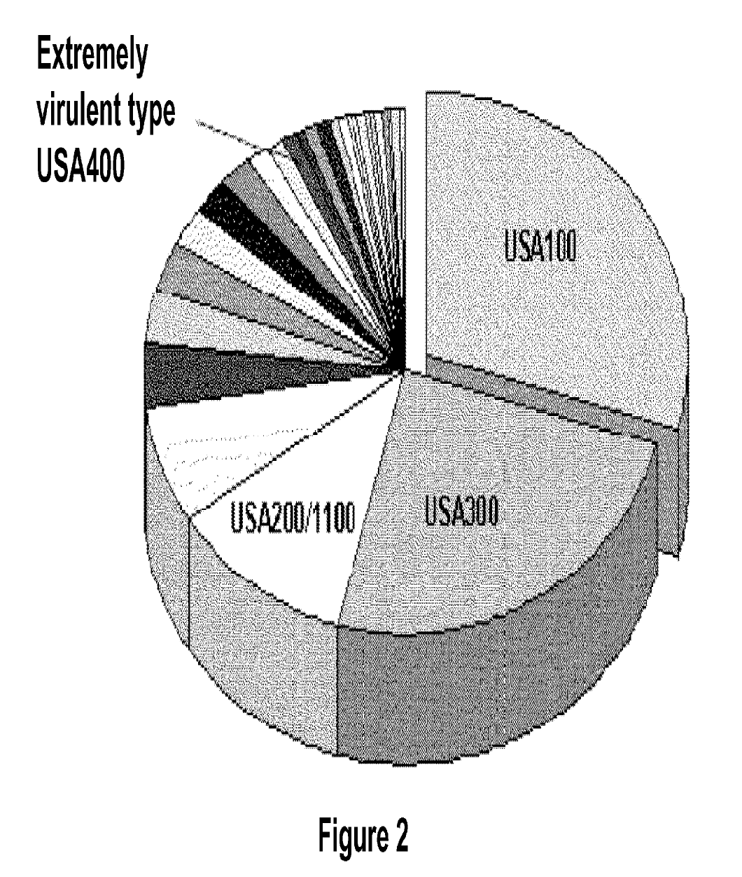

A total of 326 human clinical Staphylococcus aureus isolate samples were obtained from the Centers for Disease Control (CDC), Johns Hopkins University and University of Arizona. These samples were tested using a combination of 16 primer pairs comprising: the eight identification/resistance/virulence primer pairs listed in Table 4 and the eight genotyping primer pairs listed in Table 8. Virulence (PVL), antibiotic resistance (to Methicilin, Erythromycin and Mupirocin), and strain type were determined for each of the 326 samples. Results are summarized in Table 11 and in FIG. 2.

TABLE 11
Identification and Determination of Virulence and Drug Resistance of 326
Clinical Isolates using Staphylococcus aureus Primer Pair Panel

| | | | | Antibiotic Resistance | | | | |
|---|---|---|---|---|---|---|---|---|
| | Identification | | Virulence | Methicillin | | Erythromycin | | Mupirocin |
| # of Isolates | tufB | nuc | PVL | mecA | MecI-R | ermA | ermC | mupR |
| 81 | S. aureus | + | − | + | + | + | − | − |
| 81 | S. aureus | + | − | + | + | − | − | − |
| 34 | S. aureus | + | − | + | + | + | − | − |
| 32 | S. aureus | + | − | + | + | − | + | − |
| 30 | S. aureus | + | + | + | + | − | − | − |
| 30 | S. aureus | + | − | + | + | − | − | − |
| 10 | S. aureus | + | − | + | + | − | + | − |
| 7 | S. aureus | + | + | − | − | − | − | − |
| 3 | S. aureus | + | − | + | + | + | − | + |

+: presence of indicated gene/virulence/resistance;
−: absence of indicated gene/virulence/resistance

As shown in FIG. 2, Staphylococcus aureus strains USA 100, USA 300, USA 200/1100, and the extremely virulent USA 400 were identified among the 326 clinical isolate using the genotyping primer pairs used in this example. The genotyping data obtained using the methods provided here were consistent with data from by the agencies that provided the samples, obtained via pulse-field gel electrophoresis and sequencing. As illustrated in Table 11, tufB and nuc primer pairs confirmed that all 326 isolates belonged to the Staphylococcus aureus species. 37 samples exhibited virulence as identified by the presence of the PVL gene (as indicated by a “+”). Resistance to the indicated antibiotics (“+”) was identified in a number of the samples. These drug resistance and virulence data were greater than 99% concordant with data from the agencies that provided the samples, obtained via standard phenotypic and PCR methods. Further, the data show that accurate and precise identification, genotype, virulence, and drug resistance information can be determined for a large group of clinical samples using a panel combining the identification, characterization and genotyping primer pairs in Examples 7 and 8. This observation suggests that a kit comprising a combination of any of the primer pairs in the panel of primer pairs used in this example, or a combination of any of the other Staphylococcus aureus primer pairs provided herein configured to hybridize within the genes in this example will be a useful embodiment.

### Example 10

### Primer Pairs for Determining Resistance and Sensitivity to Quinolones

Table 12 illustrates four primer pairs that were configured to determine quinolone resistance or sensitivity of Staphylococcus aureus bioagents. The primers of these pairs were configured to hybridize within regions of the Staphylococcus aureus gyrA gene. Sequences for these primers can be found in Table 1, and the primers are cross-referenced by primer name and SEQ ID NO. in Table 12.

TABLE 12
Primer Pairs for Identification of Quinolone
Resistance in Staphylococcus aureus

| | | Forward | | Reverse |
|---|---|---|---|---|
| Primer | | Primer | | Primer |
| Pair | Forward | SEQ ID | Reverse | SEQ ID |
| Number | Primer Name | NO. | Primer Name | NO. |
| 2738 | GYRA_NC002 | 2 | GYRA_NC002 | 5 |
| | 953-7005- | | 953-7005- | |
| | 9668_166_195_F | | 9668_265_287_R | |
| 2739 | GYRA_NC002 | 3 | GYRA_NC002 | 6 |
| | 953-7005- | | 953-7005- | |
| | 9668_221_249_F | | 9668_316_343_R | |
| 2740 | GYRA_NC002 | 3 | GYRA_NC002 | 7 |
| | 953-7005- | | 953-7005- | |
| | 9668_221_249_F | | 9668_253_283_R | |
| 2741 | GYRA_NC002 | 4 | GYRA_NC002 | 5 |
| | 953-7005- | | 953-7005- | |
| | 966_8234_261_F | | 9668_265_287_R | |

Each of the primer pairs listed in Table 12 is configured to generate an amplicon within at least a portion of the QRDR region of the gyrA gene (SEQ ID NO.:10), which confers quinolone resistance or sensitivity. The QRDR comprises the position of a drug resistance-conferring SNP of the gyrA gene sequence, comprising a change of a single “C” nucleobase to a “T” nucleobase that results in a leucine instead of a serine at amino acid of the gyrase A protein. In the case of the reference sequence used to configure the primer pairs of Table 12, the SNP is located at position 251 of the extraction sequence ((coordinates 7005-9668) SEQ ID NO.: 8), which is the gyrA gene, from GenBank gi number 49484912. Forward primers in Table 12 are configured to comprise sequence identity within SEQ ID NO.: 11, a region of GenBank gi number 49484912. The reverse primers in Table 12 are configured to comprise reverse complementarity within SEQ ID NO.: 12, another region of GenBank gi number 49484912. The gyrA primer pairs provided in Table 12, when used in the methods provided herein, can detect a single nucleotide change at this SNP position, and are thus able to determine the drug resistant/sensitive genotype for the gyrA gene for a given Staphylococcus aureus bioagent.

### Example 11

### Characterizing Staphylococcus aureus in a Patient Sample Using Quinolone Resistant Primer Pairs and Other Staphylococcus aureus Primer Pairs

Population genotypes for mixed populations of bioagents can be identified with high sensitivity by PCR-ESI/MS because amplified bioagent nucleic acids having different base compositions appear in different positions in the mass spectrum. The dynamic range for mixed PCR-ESI/MS detections has previously been determined to be approximately 100:1 (Hofstadler, S. A. et al., Inter. J. Mass Spectrom. (2005) 242, 23), which allows for detection of genotype variants with as low as 1% abundance in a mixed population. This detection using PCR-ESI/MS surveillance does not require secondary testing.

A wound sample from a patient infected with Staphylococcus aureus was analyzed directly by the methods provided herein using a panel of 17 primer pairs comprising: the eight identification/resistance/virulence primer pairs listed in Table 4, the eight genotyping primer pairs listed in Table 8, and the quinolone resistance determining primer pair (number 2740, SEQ ID NO: 3:SEQ ID NO:7) listed in Table 12.

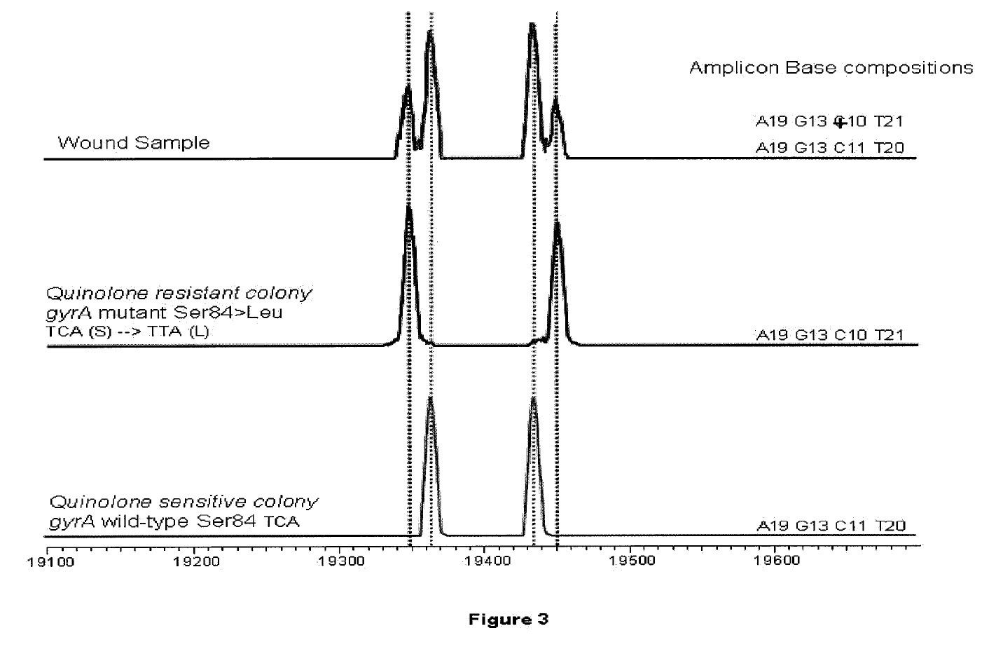

The sample was analyzed directly as described above in the previous examples using the primer pairs of Table 4, 8, and 12 (listed along the top of Table 13) in the methods provided herein. Further, a portion of the sample was cultured on an agar plate over a period of 2 days for further testing. Following the two-day culture, 9 colonies were picked and nucleic acids there from analyzed by the 17 primer pairs described above using the methods provided herein. The results are summarized in Table 13 and FIG. 3.

TABLE 13
Analysis of Patient Sample Comprising Mixed Population of Staphylococcus aureus
Bioagents: Identification of Quinolone Resistant and Sensitive Genotypes

| | | | | | Antibiotic Resistance | | | | | | |
|---|---|---|---|---|---|---|---|---|---|---|---|
| | | | | | Methicillin | | | | | | |
| | ID | | Virulence | | | pp # | Erythromycin | | Mupirocin | Quinolone | Strain |
| | pp # | pp # | pp # | pp # | pp # | 2056 | pp # | pp # | pp | pp # | Type |
| | 2249 | 2256 | 2095 | 2095 | 879 | MecI- | 2081 | 2086 | #2313 | 2740 | panel of |
| | tufB | nuc | lukD | PVL | mecA | R | ermA | ermC | mupR | gyrA | Table 8 |
| Wound | SA | + | + | + | + | + | − | − | − | 75%− | USA300 |
| | | | | | | | | | | 25%+ | |
| Colony 1 | SA | + | + | + | + | + | − | − | − | − | USA300 |
| Colony 2 | SA | + | + | + | + | + | − | − | − | − | USA300 |
| Colony 3 | SA | + | + | + | + | + | − | − | − | + | USA300 |
| Colony 4 | SA | + | + | + | + | + | − | − | − | − | USA300 |
| Colony 5 | SA | + | + | + | + | + | − | − | − | − | USA300 |
| Colony 6 | SA | + | + | + | + | + | − | − | − | − | USA300 |
| Colony 7 | SA | + | + | + | + | + | − | − | − | − | USA300 |
| Colony 8 | SA | + | + | + | + | + | − | − | − | + | USA300 |
| Colony 9 | SA | + | + | + | + | + | − | − | − | − | USA300 |

ID: Identification;
pp#: primer pair number;
SA: Staphylococcus aureus;
+: presence of indicated gene/virulence/resistance;
−: absence of indicated gene/virulence/resistance

As shown in Table 13, the wound sample, and all colonies grown from that sample were determined to comprise one or more bioagents, identified by the methods provided here as Strain USA300 of MRSA Staphylococcus aureus. These one or more bioagents comprised in all samples were also determined to be viurulent (pvl, lukD), methicillin resistant (mecA, mecl-R), and sensitive to erythromycin and mupirocin (ermA, ermC, mupR).

However, use of primer pair # 2740, which is configured to generate amplicons within the gyrA gene, identified a mixed population of bioagents in the patient sample, with more than one distinguishable genotype for the gyrA gene. FIG. 3 shows a mass spectrum for the sample generated using primer pair number 2740. The two peak groupings represent the forward and reverse strands of the amplicon. Two different base compositions for amplicons generated by the primer pair were identified in the sample, evidenced by the double peaks shown for each strand. These double peaks (and base compositions determined therefrom) indicate that two genotypes, differing only by a single nucleotide at a SNP position in gyrA, were present in the patient sample. One genotype, comprising a C at the SNP of the gyrA gene, conferring quinolone sensitivity, resulted in an amplicon with the base composition A.sub.19 G.sub.13 C.sub.11 T.sub.20. The other, comprising a T at the SNP position, conferring quinolone resistance, resulted in an amplicon with the base composition: A.sub.19 G.sub.13 C.sub.10 T.sub.21. As shown in the spectrum, the lower abundance genotype was present at approximately 25% of the population. This result is also indicated in Table 13, which lists the population genotype for the gyrA gene (Quinolone column), which comprises both quinolone resistant and quinolone sensitive genotypes at 25 and 75% respectively.

Further, Table 13 shows that two of the nine colonies (colony 3 and 8) screened in this example were found to comprise quinolone resistance, while the other six colonies comprised quinolone sensitivity, supporting the finding that the double peaks in the spectrum for the wound sample represent a mixed population with two distinguishable genotypes. A spectrum and a base composition for an example of each type of colony is also shown in FIG. 3.

Thus, the primer pairs and methods used in this example identified a mixed population of Staphylococcus aureus bioagents in a patient sample, and identified the population genotype for this mixed population. The methods and primer pairs provided herein will likely be useful in identifying population genotypes, emerging genotypes, and emerging populations of bioagents. A kit comprising a combination of any of the primer pairs used in this example or other gyrA primer pairs provided herein will likely be a useful embodiment.

### Example 12

### Periodic Analysis of Population Genotypes in a Sample over time

A sample, obtained from a patient or other sample source will be monitored over time using the primer pairs provided herein configured to identify quinolone resistant or sensitive genotypes. In this example, nucleic acids from the sample, obtained from a patient or other source suspected of comprising one or more bioagents, will be amplified using one or more of the primer pairs from Table 12, from each of any Staphylococcus aureus bioagents comprised in the sample. A base composition and/or molecular mass obtained using the methods provided herein will be compared to a database comprising molecular masses and/or base compositions, each indexed to the primer pair used and a bioagent genotype. Thus, a population genotype will be identified for the gyrA gene that will indicate the presence or absence of quinolone resistant and/or sensitive Staphylococcus aureus bioagents in the sample source. Optionally, one or more additional primer pairs will be used, such as any of the primer pairs from Tables 4-6 and 8-9 will be used to determine other characteristics of the bioagents in the sample.

An antibiotic regimen tailored to the identified genotype or genotypes will then be administered to the sample source. If the population comprises only the quinolone sensitive genotype, the antibiotic regimen may comprise a quinolone. If at least a percentage of the bioagents in the population of bioagents in the sample source comprises the quinolone resistant genotype, the antibiotic regimen will comprise an antibiotic for treating quinolone resistant bacteria. Periodically, samples will be subsequently obtained from the source, and the method repeated to monitor for emerging genotypes. Following each periodic repeat of the method, it will be determined whether there is an emerging genotype in the population of bioagents in the sample. If, after the initial identification, quinolones are being used in the antibiotic regimen tailored to treat the sample source and an emerging quinolone resistant genotype is identified during the periodic testing, the regimen will be modified to treat quinolone resistant bacteria. This modification will comprise addition of an antibiotic for treating quinolone resistant bacteria, and may further comprise discontinuation of treatment with quinolones. In one embodiment, a combination of quinolones and an antibiotic to treat quinolone resistant bacteria may be used.

Various modifications to the description herein will be apparent to those skilled in the art from the foregoing description. Such modifications fall within the spirit and scope of the current invention and appended claims. Each reference (including, but not limited to, journal articles, U.S. and non-U.S. patents, patent application publications, international patent application publications, gene bank accession numbers, internet web sites, and the like) cited in the present application is incorporated herein by reference in its entirety.

What is claimed is:

1. A method for identifying a population genotype comprising the steps of:
(a) obtaining a sample suspected of comprising a population of bioagents;
(b) amplifying a nucleic acid from each of two or more bioagents from said population of bioagents in said sample using a primer pair that is configured to generate an amplicon from within a region defined by SEQ ID NO: 10, thereby generating amplicons from said nucleic acids;
(c) determining a molecular mass measurement for each of said amplicons using a mass spectrometer;
(d) calculating a base composition from each molecular mass measurement; and
(e) identifying a population genotype for said population of bioagents by comparing each of said base compositions calculated in step (d) to a database of base compositions indexed to the primer pair of step (b) and a known bioagent genotype.

2. The method of claim 1 wherein said primer pair further comprises a forward member that is 20 to 35 nucleobases in length and comprises at least 80% identity to a first portion of SEQ ID NO: 10 and a reverse member that is 20 to 35 nucleobases in length and comprises at least 80% reverse complementarity to a second portion of SEQ ID NO: 10.

3. The method of claim 2 wherein said forward member comprises at least 90% identity to said first portion of SEQ ID NO: 10.

4. The method of claim 2 wherein said forward member comprises at least 95% identity to said first portion of SEQ ID NO: 10.

5. The method of claim 2 wherein said forward member comprises at least 97% identity to said first portion of SEQ ID NO: 10.

6. The method of claim 2 wherein said forward primer pair member comprises SEQ ID NO: 2 with 0-8 nucleobase deletions, additions and/or substitutions.

7. The method of claim 2 wherein said forward primer pair member comprises SEQ ID NO: 3 with 0-8 nucleobase deletions, additions and/or substitutions.

8. The method of claim 2 wherein said forward primer pair member comprises SEQ ID NO: 4 with 0-8 nucleobase deletions, additions and/or substitutions.

9. The method of claim 2 wherein said reverse member comprises at least 90% reverse complementarity to said second portion of SEQ ID NO: 10.

10. The method of claim 2 wherein said reverse member comprises at least 95% reverse complementarity to said second portion of SEQ ID NO: 10.

11. The method of claim 2 wherein said reverse member comprises at least 97% reverse complementarity to said second portion of SEQ ID NO: 10.

12. The method of claim 2 wherein said reverse primer pair member comprises SEQ ID NO: 5 with 0-6 nucleobase deletions, additions and/or substitutions.

13. The method of claim 2 wherein said reverse primer pair member comprises SEQ ID NO: 6 with 0-8 nucleobase deletions, additions and/or substitutions.

14. The method of claim 2 wherein said reverse primer pair member comprises SEQ ID NO: 7 with 0-9 nucleobase deletions, additions and/or substitutions.

15. The method of claim 1 wherein either or both of said primer members comprises at least one modified nucleobase.

16. The method of claim 15 wherein said modified nucleobase is a mass modified nucleobase.

17. The method of claim 16 wherein said modified nucleobase is 5-Iodo-C.

18. The method of claim 15 wherein said modified nucleobase is a universal nucleobase.

19. The method of claim 18 wherein said modified nucleobase is inosine.

20. The method of claim 1 wherein either or both of said primer members comprise a non-templated 5′ T-residue.

21. The method of claim 1 wherein said population of bioagents comprises at least two bacteria belonging to the Staphylococcus genus.

22. The method of claim 21 wherein at least one of said bacteria is resistant to quinolone antimicrobial therapy.

23. The method of claim 21 wherein at least one of said bacteria is resistant to quinolone antimicrobial therapy and at least one of said bacteria is sensitive to quinolone antimicrobial therapy.

24. The method of claim 1 wherein said population of bioagents comprises at least two bacteria belonging to the Staphylococcus aureus species.

25. The method of claim 24 wherein at least one of said bacteria is resistant to quinolone antimicrobial therapy.

26. The method of claim 24 wherein at least one of said bacteria is resistant to quinolone antimicrobial therapy and at least one of said bacteria is sensitive to quinolone antimicrobial therapy.

27. The method of claim 1 wherein an antibiotic regimen tailored to treat the identified genotypes for the population of bioagents is delivered to the sample source.

28. The method of claim 1 wherein steps (a) to (e) are periodically repeated.

29. A method of reducing a population of bacteria in a person needing such a treatment comprising the steps of:
(a) obtaining from a person a sample suspected of comprising a population of bacterial bioagents;
(b) amplifying a nucleic acid from each of two or more bacterial bioagents in said sample using a primer pair that is configured to generate an amplicon from within a region of defined by SEQ ID NO: 10, thereby generating amplicons from said nucleic acids;
(c) determining a molecular mass measurement for each of said amplicons using a mass spectrometer;
(d) calculating a base composition from each molecular mass measurement;
(e) identifying a population genotype for said population of bioagents by comparing each of said base compositions calculated in step (d) to a database of base compositions indexed to the primer pair of step (b) and a known bioagent genotype; and
(f) administering to a person in need thereof an antibiotic regimen tailored to treat the identified genotypes for the population of bacterial bioagents.

30. The method of claim 29 wherein said primer pair further comprises a forward member that is 20 to 35 nucleobases in length and comprises at least 80% identity to a first portion of SEQ ID NO: 10 and a reverse member that is 20 to 35 nucleobases in length and comprises at least 80% reverse complementarity to a second portion of SEQ ID NO: 10.

31. The method of claim 30 wherein said forward member comprises at least 90% identity to said first portion of SEQ ID NO: 10.

32. The method of claim 30 wherein said forward member comprises at least 95% identity to said first portion of SEQ ID NO: 10.

33. The method of claim 30 wherein said forward member comprises at least 97% identity to said first portion of SEQ ID NO: 10.

34. The method of claim 30 wherein said forward primer pair member comprises SEQ ID NO: 2 with 0-8 nucleobase deletions, additions and/or substitutions.

35. The method of claim 30 wherein said forward primer pair member comprises SEQ ID NO: 3 with 0-8 nucleobase deletions, additions and/or substitutions.

36. The method of claim 30 wherein said forward primer pair member comprises SEQ ID NO: 4 with 0-8 nucleobase deletions, additions and/or substitutions.

37. The method of claim 30 wherein said reverse member comprises at least 90% reverse complementarity to said second portion of SEQ ID NO: 10.

38. The method of claim 30 wherein said reverse member comprises at least 95% reverse complementarity to said second portion of SEQ ID NO: 10.

39. The method of claim 30 wherein said reverse member comprises at least 97% reverse complementarity to said second portion of SEQ ID NO: 10.

40. The method of claim 30 wherein said reverse primer pair member comprises SEQ ID NO: 5 with 0-6 nucleobase deletions, additions and/or substitutions.

41. The method of claim 30 wherein said reverse primer pair member comprises SEQ ID NO: 6 with 0-8 nucleobase deletions, additions and/or substitutions.

42. The method of claim 30 wherein said reverse primer pair member comprises SEQ ID NO: 7 with 0-9 nucleobase deletions, additions and/or substitutions.

43. The method of claim 30 wherein either or both of said primer members comprises at least one modified nucleobase.

44. The method of claim 43 wherein said modified nucleobase is a mass modified nucleobase.

45. The method of claim 44 wherein said modified nucleobase is 5-Iodo-C.

46. The method of claim 43 wherein said modified nucleobase is a universal nucleobase.

47. The method of claim 46 wherein said modified nucleobase is inosine.

48. The method of claim 29 wherein either or both of said primer members comprise a non-templated 5′ T-residue.

49. The method of claim 29 wherein said population of bacterial bioagents comprises at least two bacteria belonging to the Staphylococcus genus.

50. The method of claim 49 wherein at least one of said bacteria is resistant to quinolone antimicrobial therapy.

51. The method of claim 49 wherein at least one of said bacteria is resistant to quinolone antimicrobial therapy and at least one of said bacteria is sensitive to quinolone antimicrobial therapy.

52. The method of claim 29 wherein said population of bacterial bioagents comprises at least two bacteria belonging to the Staphylococcus aureus species.

53. The method of claim 52 wherein at least one of said bacteria is resistant to quinolone antimicrobial therapy.

54. The method of claim 52 wherein at least one of said bacteria is resistant to quinolone antimicrobial therapy and at least one of said bacteria is sensitive to quinolone antimicrobial therapy.

55. The method of claim 29 wherein steps (a) to (e) are periodically repeated.

56. The method of claim 55 wherein an emerging genotype is identified in step (e) of one or more of said periodic repeats, further comprising modifying said antibiotic regimen to treat said emerging genotype.

57. The method of claim 29 wherein said antibiotic regimen comprises an antibiotic for treating quinolone resistant bacteria and an antibiotic for treating quinolone sensitive bacteria.

58. A composition of matter comprising a purified oligonucleotide primer pair wherein each primer member of said primer pair is 20 to 35 nucleobases in length and wherein the forward primer comprises at least 80% identity with a first portion of SEQ ID NO: 10 and the reverse primer comprises at least 80% reverse complementarity with a second portion of SEQ ID NO: 10.

59. The composition of claim 58 wherein the forward member comprises at least 90% identity to said first portion of SEQ ID NO: 10.

60. The composition of claim 58 wherein the forward member comprises at least 95% identity to said first portion of SEQ ID NO: 10.

61. The composition of claim 58 wherein the forward member comprises at least 97% identity to said first portion of SEQ ID NO: 10.

62. The composition of claim 58 wherein the forward primer pair member comprises SEQ ID NO: 2 with 0-8 nucleobase deletions, additions and/or substitutions.

63. The composition of claim 58 wherein the forward primer pair member comprises SEQ ID NO: 3 with 0-8 nucleobase deletions, additions and/or substitutions.

64. The composition of claim 58 wherein the forward primer pair member comprises SEQ ID NO: 4 with 0-8 nucleobase deletions, additions and/or substitutions.

65. The composition of claim 58 wherein the forward primer pair member comprises at least 80% identity with a portion of SEQ ID NO: 11.

66. The composition of claim 58 wherein the reverse member comprises at least 90% reverse complementarity to said second portion of SEQ ID NO: 10.

67. The composition of claim 58 wherein the reverse member comprises at least 95% reverse complementarity to said second portion of SEQ ID NO: 10.

68. The composition of claim 58 wherein the reverse member comprises at least 97% reverse complementarity to said second portion of SEQ ID NO: 10.

69. The composition of claim 58 wherein the reverse primer pair member comprises SEQ ID NO: 5 with 0-6 nucleobase deletions, additions and/or substitutions.

70. The composition of claim 58 wherein the reverse primer pair member comprises SEQ ID NO: 6 with 0-8 nucleobase deletions, additions and/or substitutions.

71. The composition of claim 58 wherein the reverse primer pair member comprises SEQ ID NO: 7 with 0-9 nucleobase deletions, additions and/or substitutions.

72. The composition of claim 58 wherein the reverse primer pair member comprises at least 80% reverse complementarity with a portion of SEQ ID NO: 12.

73. The composition of claim 58 wherein either or both of the primer members comprises at least one modified nucleobase.

74. The composition of claim 73 wherein the modified nucleobase is a mass modified nucleobase.

75. The composition of claim 74 wherein the modified nucleobase is 5-Iodo-C.

76. The composition of claim 73 wherein the modified nucleobase is a universal nucleobase.

77. The composition of claim 76 wherein the modified nucleobase is inosine.

78. The composition of claim 58 wherein either or both of the primer members comprise a non-templated 5′ T-residue.

79. The composition of claim 58 wherein said primer pair is configured to generate an amplicon of between about 45 and about 192 nucleobases in length comprising a region of SEQ ID NO: 10.
